(19) United States
(12) Patent Application Publication
(10) Pub. No.: US 2026/0315768 A1
(43) Pub. Date: Oct. 15, 2026

(54) DEVICES, SYSTEMS, AND METHODS FOR MONITORING AND TREATING DISEASES

(71) Applicant: Fenestra Medical Technologies, Inc., Needham, MA (US)

(72) Inventors: Marc D. Friedman, Needham, MA (US); Barry G. Morse, Cotuit, MA (US); Jonathan L. Friedman, Needham, MA (US)

(21) Appl. No.: 19/481,100

(22) PCT Filed: May 3, 2024
(86) PCT No.: PCT/US2024/027866

§ 371 Date: Nov. 3, 2025

Related U.S. Application Data
(60) Provisional application No. 63/463,757, filed on May 3, 2023.
(60) Provisional application No. 63/536,299, filed on Sep. 1, 2023.
(60) Provisional application No. 63/546,098, filed on Oct. 27, 2023.
(60) Provisional application No. 63/621,326, filed on Jan. 16, 2024.

(51) Int. Cl. A61M 39/02; A61B 17/00; A61B 17/34; A61B 90/00; A61L 2/03; A61L 103/15

(52) U.S. Cl. CPC A61M 39/0247; A61B 17/3423; A61B 90/36; A61L 2/03; A61B 2017/00022; A61B 2017/00557; A61B 2017/00862; A61B 2017/3425; A61L 2103/15; A61M 2039/0205; A61M 2039/0255; A61M 2039/0261; A61M 2039/0267; A61M 2039/027; A61M 2039/0276; A61M 2039/0279; A61M 2039/0282; A61M 2039/0285; A61M 2039/0288; A61M 2039/0297; A61M 2202/0007; A61M 2202/02; A61M 2202/04; A61M 2205/0216; A61M 2205/04; A61M 2205/051; A61M 2205/058; A61M 2205/3303; A61M 2205/3306; A61M 2210/1042

(57) ABSTRACT

In an example, a percutaneous port implant system (PPIS) is described. The PPIS includes a port housing. The port housing includes a proximal housing portion defining a proximal opening at a proximal end of the port housing, a distal housing portion defining a distal opening at a distal end of the port housing, a percutaneous body portion extending between the proximal housing portion and the distal housing portion. The percutaneous body portion is configured to engage a wall of a body cavity of a human body while the proximal housing portion is positioned external to the body cavity and the distal housing portion is positioned within an interior of the body cavity. The port housing also includes a port lumen extending between the proximal opening and the distal opening. The port lumen extends entirely through the proximal housing portion, the percutaneous body portion, and the distal housing portion.

### CROSS-REFERENCE & INCORPORATION BY REFERENCE

This application claims the benefit of priority of U.S. Provisional Application No. 63/463,757, filed May 3, 2023, and U.S. Provisional Application No. 63/536,299, filed Sep. 1, 2023, and U.S. Provisional Application No. 63/546,098, filed on Oct. 27, 2023, U.S. Provisional Application No. 63/621,326, filed on Jan. 16, 2024, the contents of which are hereby incorporated by reference in their entirety.

Additionally, all patents, applications, and publications cited in this disclosure are hereby incorporated by reference in their entirety.

### FIELD

The present application relates generally to devices, systems, and methods for monitoring and treating diseases. More particularly, the present application relates to implant devices, systems, and methods for providing access to a body cavity of a human body.

### BACKGROUND

Many existing technologies are designed for specific purposes and look to minimize the types and numbers of invasive procedures performed on a patient. Each individual technology may require several invasive procedures and the combinations of these procedures make it difficult or impossible to implement them all individually. Combinations of therapies and diagnostics generally help to achieve the improved results (remissions/cures) for treating cancer of the individual patient. Every cancer type and disease state and every person who has that form of cancer or disease state is unique based on their age, sex, lifestyle, genetic disposition and the stage of the cancer or disease state when first diagnosed. Successful treatment often lies in the ability to both locally and systemically deliver both therapeutics and/or diagnostics with enough frequency and to ascertain through data collected, whether the individual patient is responding to their treatment plan and/or responding to their treatment plan with few or the least number of side effects.

In today's armamentarium for the successful treatment of cancer and other forms of disease states, there are a multitude of different combinations of diagnostic and treatment modalities depending on the targeted cancer or disease state. Disease states such as cancer are treated over long periods of time often several months to years and require multiple treatments, multiple combinations of treatment modalities (e.g., chemotherapy, radiation therapy, immunotherapy, photodynamic therapy, thermal therapy, electrical stimulation therapy, etc.) and multiple diagnostic tests to monitor the progression of disease. It is the combinations of therapies and diagnostics that generally achieve the best results (e.g., remissions/cures) for the individual patient. There is rarely a single treatment modality that is a cure-all.

### BRIEF DESCRIPTION OF THE FIGURES

The novel features believed characteristic of the illustrative embodiments are set forth in the appended claims. The illustrative embodiments, however, as well as a preferred mode of use, further objectives, and descriptions thereof, will best be understood by reference to the following detailed description of an illustrative implementation of the present disclosure when read in conjunction with the accompanying figures.

FIG. 1A depicts a simplified block diagram of a system including a percutaneous port implant system in an open configuration, according to an example.

FIG. 1B depicts a simplified block diagram of the system shown in FIG. 1A with the percutaneous port implant system in a closed configuration, according to an example.

FIG. 1C depicts a simplified block diagram of a system shown in FIG. 1A including the percutaneous port implant system in a closed and capped configuration, according to an example.

FIG. 2A depicts a perspective view of an implementation of the percutaneous port implant system in the open configuration, according to an example.

FIG. 2B depicts a perspective view of a plug catheter, according to an example.

FIG. 2C depicts a perspective view of an implementation of the percutaneous port implant system of FIG. 2A in the closed configuration, according to an example.

FIG. 2D depicts a perspective view of an implementation of the percutaneous port implant system of FIG. 2C in the closed and capped configuration, according to an example.

FIG. 2E depicts an implementation of the percutaneous port implant system positioned implanted in a human body and an endoscope accessing a body cavity of the human body through the percutaneous port implant system, according to an example.

FIG. 2F depicts an implementation of the percutaneous port implant system positioned implanted in the human body and a plug catheter accessing the body cavity of the human body through the percutaneous port implant system, according to an example.

FIG. 2G depicts an implementation of the percutaneous port implant system positioned implanted in the human body with a cap coupled to the percutaneous port implant system, according to an example.

FIG. 3A depicts a perspective view of an implementation of the percutaneous port implant system, according to another example.

FIG. 3B depicts a perspective view of an implementation of the percutaneous port implant system, according to another example.

FIG. 4A depicts a distal port catheter end of the percutaneous port implant system including one or more suture haptics, according to an example.

FIG. 4B depicts a distal port catheter end of the percutaneous port implant system including one or more suture haptics, according to another example.

FIG. 4C depicts a distal port catheter end of the percutaneous port implant system including one or more suture haptics, according to another example.

FIG. 4D depicts a distal port catheter end of the percutaneous port implant system including one or more suture haptics, according to another example.

FIG. 4E depicts a distal port catheter end of the percutaneous port implant system including one or more suture haptics, according to another example.

FIG. 5 depicts a partial cross-sectional view of an implementation of the percutaneous port implant system, according to another example.

FIG. 6A depicts a partial cross-sectional view of an implementation of the percutaneous port implant system, according to another example.

FIG. 6B depicts a partial cross-sectional view of an implementation of the percutaneous port implant system, according to another example.

FIG. 6C depicts a partial cross-sectional view of an implementation of the percutaneous port implant system, according to another example.

FIG. 7A depicts a partial cross-sectional view of an implementation of an axial retention mechanism of the percutaneous port implant system, according to another example.

FIG. 7B depicts a partial cross-sectional view of an implementation of an axial retention mechanism of the percutaneous port implant system, according to another example.

FIG. 7C depicts a partial cross-sectional view of an implementation of an axial retention mechanism of the percutaneous port implant system, according to another example.

FIG. 8A depicts a simplified partial cross-sectional view of a plug insertion tool, according to an example.

FIG. 8B depicts a simplified partial cross-sectional view of a plug catheter insertion assembly, according to an example.

FIG. 8C depicts a simplified partial cross-sectional view of a plug catheter insertion assembly in an undeployed state and a deployed state, according to an example.

FIG. 8D depicts a simplified partial cross-sectional view of a plug catheter insertion assembly in an undeployed state and a deployed state, according to an example.

FIG. 9A depicts a simplified view of an over bandage, according to another example.

FIG. 9B depicts a perspective view of a cap, according to another example.

FIG. 9C depicts a perspective view of a fill rod, according to another example.

FIG. 10A depicts a simplified partial cross-sectional view of a bayonet connector system in a first position, according to another example.

FIG. 10B depicts a simplified partial cross-sectional view of a bayonet connector system in a second position, according to another example.

FIG. 10C depicts a simplified proximal view of a bayonet connector system in a first and second position, according to another example.

FIG. 11 depicts a simplified view of the plug catheter distal end and the distal port catheter end with the bayonet connector system, according to another example.

FIG. 12A depicts a simplified cross-sectional view of a shortened percutaneous port implant system, according to another example.

FIG. 12B depicts an enlarged view of a portion of the shortened percutaneous port implant system shown in FIG. 12A, according to another example.

FIG. 13A depicts a perspective view of a multiport catheter and multi plug catheter configuration of the percutaneous port implant system, according to another example.

FIG. 13B depicts a cross sectional view of a multiport catheter and multi plug catheter configuration of the percutaneous port implant system, according to another example.

FIG. 14A depicts a perspective view of a fenestrated tube plug catheter, according to an example.

FIG. 14B depicts a perspective view of an implementation of the percutaneous port implant system including the fenestrated tube plug catheter of FIG. 14A, according to an example.

FIG. 14C depicts a perspective view of a fenestrated tube plug catheter with two fenestrated portions, according to another example.

FIG. 14D depicts a perspective view of a fenestrated tube plug catheter insertion assembly, according to another example.

FIG. 14E depicts a perspective view of a fenestrated tube plug catheter insertion assembly illustrating an insertion of the process of the percutaneous port implant system, according to another example.

FIG. 15A depicts a perspective view of an extensible plug catheter, according to an example.

FIG. 15B depicts a perspective view of an implementation of the percutaneous port implant system in the closed configuration including the extensible plug catheter of FIG. 15A, according to another example.

FIG. 15C depicts a perspective view of an extensible plug catheter insertion assembly, according to an example.

FIG. 15D depicts a perspective view of an extensible plug catheter insertion assembly illustrating an insertion of the process of the percutaneous port implant system, according to another example.

FIG. 16A depicts a perspective view of a shortened fenestrated tube plug catheter, according to another example.

FIG. 16B depicts a perspective view of an implementation of the percutaneous port implant system including a shortened fenestrated tube plug catheter, according to another example.

FIG. 16C depicts a perspective view of a shortened extensible plug catheter, according to another example.

FIG. 16D depicts an implementation of the percutaneous port implant system including a shortened extensible plug catheter, according to another example.

FIG. 17A depicts a perspective view of a distal end of a percutaneous port implant system, according to another example.

FIG. 17B depicts a perspective view of a distal end of a percutaneous port implant system with an extensible plug catheter, according to another example.

FIG. 18 depicts a perspective view of the percutaneous port implant system with a closed distal end, according to another example.

FIG. 19A depicts a simplified partial perspective view of an inner lumen applicator, according to an example.

FIG. 19B depicts a simplified partial perspective view of an inner lumen applicator, according to another example.

FIG. 19C depicts a simplified partial perspective view of an inner lumen applicator with a lumen fixation element, according to another example.

FIG. 20A depicts a simplified partial cross-sectional view of the inner lumen applicator delivering a device, according to another example.

FIG. 20B depicts a simplified partial cross-sectional view of the inner lumen applicator delivering a device, according to another example.

FIG. 20C depicts a simplified partial cross-sectional view of the inner lumen applicator delivering a device, according to another example.

FIG. 20D depict a simplified partial cross-sectional view of the inner lumen applicator delivering a device s, according to another example.

FIG. 20E depicts a simplified partial cross-sectional view of the inner lumen applicator delivering a device, according to another example.

FIG. 20F depicts a simplified partial cross-sectional view of the inner lumen applicator delivering a device, according to another example.

FIG. 20G depicts a simplified partial cross-sectional view of the inner lumen applicator delivering a device, according to another example.

FIG. 20H depicts a simplified partial cross-sectional view of the inner lumen applicator delivering a device, according to another example.

FIG. 20I depicts a simplified partial cross-sectional view of the inner lumen applicator delivering a device, according to another example.

FIG. 21A depicts a simplified partial cross-sectional view of a Brachytherapy Needle device, according to another example.

FIG. 21B depicts a simplified partial cross-sectional view of a Brachytherapy Needle device in a housing, according to another example.

FIG. 21C depicts a simplified partial cross-sectional view of a Brachytherapy Needle device needle extending through a housing, according to another example.

FIG. 22A depicts a simplified partial cross-sectional view of an Enface Brachytherapy delivery device, according to another example.

FIG. 22B depicts a simplified partial cross-sectional view of an Enface Brachytherapy delivery device, according to another example.

FIG. 22C depicts a simplified perspective view of an Enface Brachytherapy delivery device with an inner lumen applicator, according to another example.

FIG. 22D depicts a partial cross-sectional view of an Enface Brachytherapy delivery device with an inner lumen applicator using the percutaneous port implant system, according to another example.

FIG. 23A depicts a simplified partial cross-sectional view of a Side-viewing Brachytherapy delivery device, according to an example.

FIG. 23B depicts a simplified partial cross-sectional view of a Side-viewing Brachytherapy delivery device, according to an example.

FIG. 23C depicts a simplified partial cross-sectional view of a Side-viewing Brachytherapy delivery device, according to an example.

FIG. 23D depicts a simplified partial cross-sectional view of a Side-viewing Brachytherapy delivery device with an inner lumen applicator, according to an example.

FIG. 23E depicts a partial cross-sectional view of a Side-viewing Brachytherapy delivery device with an inner lumen applicator using the percutaneous port implant system, according to another example.

FIG. 24A depicts a simplified schematic of a multiplexing demultiplexing and thermal bladder system percutaneous port implant system, according to an example.

FIG. 24B depicts a simplified schematic of a multiplexing demultiplexing and thermal bladder system of a plug catheter of a percutaneous port implant system, according to an example.

### DETAILED DESCRIPTION

Disclosed examples will now be described more fully hereinafter with reference to the accompanying drawings, in which some, but not all of the disclosed examples are shown. Indeed, several different examples may be described and should not be construed as limited to the examples set forth herein. Rather, these examples are described so that this disclosure will be thorough and complete and will fully convey the scope of the disclosure to those skilled in the art.

By the term “approximately” or “substantially” with reference to amounts or measurement values described herein, it is meant that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

Surgical placement and implementation of short-, mid-, and long-term diagnostic and therapeutic probes and delivery lumens/catheters for a patient and their healthcare team is not trivial, especially if several different catheter types are required for a patient's treatment each through their own insertion point. A significant barrier for catheter implantation is catheter related infections. Tissue colonization because of microbial biofilms is a significant and recurrent issue of catheter related infections. Once a biofilm matrixome composed of extracellular polymeric substances has been established, they are difficult to eradicate because of antibiotic resistance through diffusion restrictions and/or chemical inactivation. The annual cost of catheter related infections worldwide is in the billions of dollars. There are three basic routes of infection and biofilm formation. The first is tunnel infection entering between the skin and the external surface of the catheter. The second is subcutaneous biofilm formation on the external surface of the catheter. The third is biofilm formation on the internal surface of the catheter especially along its distal tip as it is isolated from the immune system of the body.

The present application provides systems and methods that can address these challenges. For example, the present disclosure provides systems and methods for an implant system that can provide minimally invasive local aseptic access for (i) the delivery of therapeutics, (ii) diagnostics, (iii) manipulation and management of a tumor microenvironment, and/or (iv) an internal software and/or external software system and database for capturing and analyzing the diagnostic and/or treatment efficacy data from the implant system. In some examples, the systems and methods can work in synergy with systemic therapeutics and diagnostics and external databases of additional patient information. The systems and methods can also provide a universal technology platform that can provide continuity of development across cell, small animal, large animal and/or human technology platforms, and implementation of these combinations of technologies for the treatment of cancer and other disease states. This disclosure utilizes pancreatic cancer as a solid tumor example, but the systems and methods of the present disclosure can be applicable to many types of solid tumors located in various cavities of the human body including, for instance, an abdominopelvic cavity, a thoracic cavity, and/or a cranial cavity.

Within examples, a Percutaneous Port Implant System (PPIS) is described. The PPIS can be a generic, multiple function diagnostic and therapeutic port and catheter-based implant system, which is implanted in a single planned surgical procedure or a reduced set of procedures. The PPIS offers a more efficient and attractive option for the patient and the Healthcare Team by providing a single or reduced number of port/catheter insertion points and minimally invasive recurring aseptic access to the body cavity.

In some examples, the PPIS can be configured to provide recurring aseptic access to one or more body cavities of the human body including, for instance, the Abdominopelvic, Thoracic, and Cranial cavities for the delivery and integration of one or more diagnostics and therapeutics. For some types of cancer, such as pancreatic cancer, the systems and methods can provide more diagnostic and treatment options for a majority of pancreatic cancer patients as they do not qualify for current surgical intervention. It can also provide alternative primary treatment options for those pancreatic cancer patients that are either borderline surgically eligible or even surgically eligible as the morbidity rate for surgical procedures such as Whipple surgery is still relatively high.

In some examples, the PPIS can include external and internal hardware for multiplexing and demultiplexing systems, electrical systems, optical systems, fluid (e.g., gas or liquids) exchange and manifolds, mechanical systems, suction/drainage systems, liquid/delivery (e.g., via pumps), insufflation systems, external access ports, diagnostic and therapeutic internal delivery lumens, local drug delivery, an aseptic maintenance technology system (AMT) and/or software for their functionality. One advantage of a multifunction PPIS is that at a minimum, it may require only one surgical implantation and one surgical explantation. Multiplexing and demultiplexing systems allow for a reduced size of the PPIS through a wall of the body cavity as well as the distribution of therapeutics and diagnostics in, on, adjacent to distant to the primary targeted tissue, and/or within the body cavity.

In some implementations, multiple primary tumors, secondary metastatic tumors, and sentinel lymph nodes can be treated directly and simultaneously. Once implanted, the PPIS can provide aseptic maintenance technology for on-going delivery and exchange of minimally invasive combinations of therapeutics and diagnostics by the Healthcare Team to be implemented over weeks, months and years. The PPIS can also provide significantly less discomfort (e.g., number of pokes and jabs) and inconvenience to the patient as several diagnostic and therapeutic procedures can be performed during a single visit. In some implementations, the PPIS can be compatible with a wide range of plug catheter and/or cap designs, which can make the PPIS agnostic to specific diagnostics or therapeutics and provide a more versatile and adaptable platform for monitoring and treating diseases. Additionally, the PPIS can be configured in multiple ways to accommodate both current technologies and future technological advances.

Within examples, the AMT of the PPIS can be configured to address all three routes of infection and biofilm formation described above. This allows for the opening, closing and recurring use of the PPIS and the ability to establish, reestablish and maintain aseptic integrity of the PPIS over weeks, months and years by inhibiting and eliminating biofilms. As described in further detail below, the AMT can use combinations of three-electrode electrochemical oxidation/reduction configurations, ultraviolet-C (UV-C) light emitting diode (LED) light illumination configurations, and/or photodynamic disinfection configurations using photosensitizer compounds with light illumination on the external and internal parts of the PPIS.

In some implementations, the AMT can include a three-electrode electrochemical system, which can include a working electrode, a counter electrode and a reference electrode on a polymer membrane or polymer membranes back-to-back which couple to the internal and external surfaces of the PPIS as will be discussed in detail later. When activated, the system creates an oxidation/reduction reaction on the surface of the electrodes with the fluids and salts of the body or with fluids and/or hydrogels which can contain salts within or on the surface of the PPIS producing various reactive oxygen species (ROS) and oxidizing compounds such as hydrogen peroxide and hypochlorous acid which inhibit and destroy biofilm formation. UV-C light disinfection of internal surfaces of the PPIS can work to damage and/or destroy nucleic acids and disrupt the DNA of the microbial pathogens. Photodynamic disinfection using photosensitizer compounds with light illumination inhibit and destroy biofilm formation by production of ROS as well.

According to additional aspects of the present disclosure, a System for Diagnostics and Therapeutics (SDT) can include the PPIS and one or more additional systems. The SDT can provide a technology platform that can integrate and extend the capabilities of the PPIS to external multifunction diagnostic and therapeutic technologies, increasing an understanding of and/or treatment options for an individual patient's condition. The SDT can also provide institutional research and commercial drug/device communities a known access and treatment delivery continuum supporting a consistent roadmap to produce breakthrough combination therapies and diagnostic tools from bench to commercial implementation across developmental cell, small and large animal research platforms.

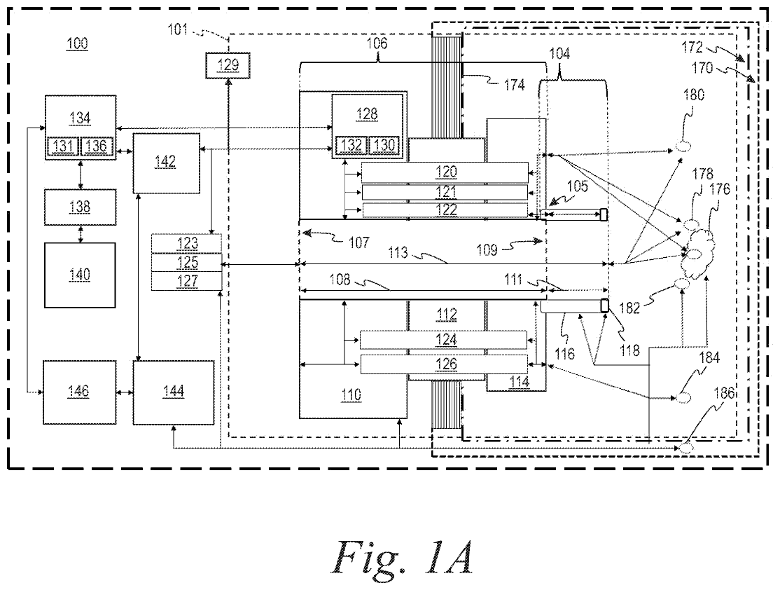

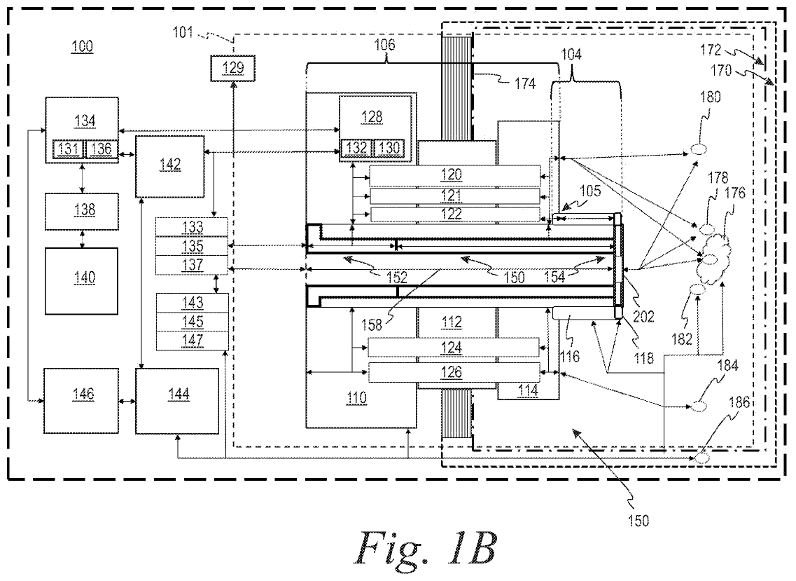

Referring now to FIGS. 1A-1C, a simplified diagram of a SDT 100 is shown according to an example. More particular, FIG. 1A depicts the SDT 100 including a PPIS 101 in an open configuration, FIG. 1B depicts the SDT 100 with the PPIS 101 in a closed configuration, and FIG. 1C depicts the PPIS 101 in a closed and capped configuration, according to the example.

As shown in FIGS. 1A-1C, the PPIS 101 includes a port housing 106 that is configured to be implanted in a body 170 of a human being. Within examples, the port housing 106 includes a proximal housing portion 110 defining a proximal opening 107 at a proximal end of the port housing 106, a distal housing portion 114 defining a distal opening at a distal end of the port housing 106, and a percutaneous body portion 112 extending between the proximal housing portion 110 and the distal housing portion 114. The percutaneous body portion 112 is configured to engage a wall 174 of a body cavity 172 of the human body 170 while the proximal housing portion 110 is positioned external to the body cavity 172 and the distal housing portion 114 is positioned within an interior of the body cavity 172 when the port housing 106 is implanted in the human body 170. As examples, the body cavity 172 can be a pelvic cavity, an abdominal cavity, a thoracic cavity, or a cranial cavity. The PPIS 101 further includes a port lumen 108 extending between the proximal opening 107 in the proximal housing portion 110 and the distal opening 109 in the distal housing portion 114. The port lumen 108 extends entirely through the proximal housing portion 110, the percutaneous body portion 112, and the distal housing portion 114.

Additionally, as shown in FIGS. 1A-1C, the PPIS 101 can include a port catheter 104 coupled to the port housing 106 and extending within the port lumen 108 of the port housing 106. The port catheter 104 includes a port tube 116 that defines an outer lumen 111 that extends between a proximal port catheter end 105 and a distal port catheter end 118. As shown in FIG. 1A, the port lumen 108 of the port housing 106 and the outer lumen 111 of the port catheter 104 define a percutaneous port implant system (PPIS) lumen 113 that extends between the proximal opening 107 of the port housing 106 and the distal port catheter end 118. In this arrangement, the PPIS lumen 113 can provide a pathway for accessing the body cavity 172 when the port housing 106 is implanted with the percutaneous body portion 112 engaging the wall 174 of the body 170, the proximal housing portion 110 external to the body cavity 172, and the distal housing portion 114 within the body cavity 172.

As shown in FIGS. 1A-1C, the distal port catheter end 118 of the port catheter 104 can extend distally of the distal housing portion 114. In some examples, the port housing 106 can be formed from a material that is substantially rigid, whereas the port tube 116 can be formed from a material that is flexible and/or elastic in at least one dimension, as described in further detail below. This arrangement of the PPIS 101 can help to maintain patency of the PPIS lumen 113 at the wall 174 of the body 170 while providing the port catheter 104 with versatility in positioning the distal port catheter end 118 in the body cavity 172 to facilitate monitoring and/or treating one or more target tissues 176 in the body cavity 172.

As examples, the port housing 106 can be formed from one or more materials selected from a group consisting of: titanium, magnesium, platinum, stainless steel, cobalt-based alloys, polycarbonate, polymethyl methacrylate, Polyethylene, Polypropylene, Polyvinyl chloride, Polyamide, Acrylonitrile butadiene styrene (ABS). Also, as examples, the port tube 116 can be formed from one or more materials selected from a group consisting of: titanium, magnesium, platinum, stainless steel, cobalt-based alloys, polycarbonate, polymethyl methacrylate, Polyethylene, Polypropylene, Polyvinyl chloride, Polyamide, Acrylonitrile butadiene styrene (ABS).

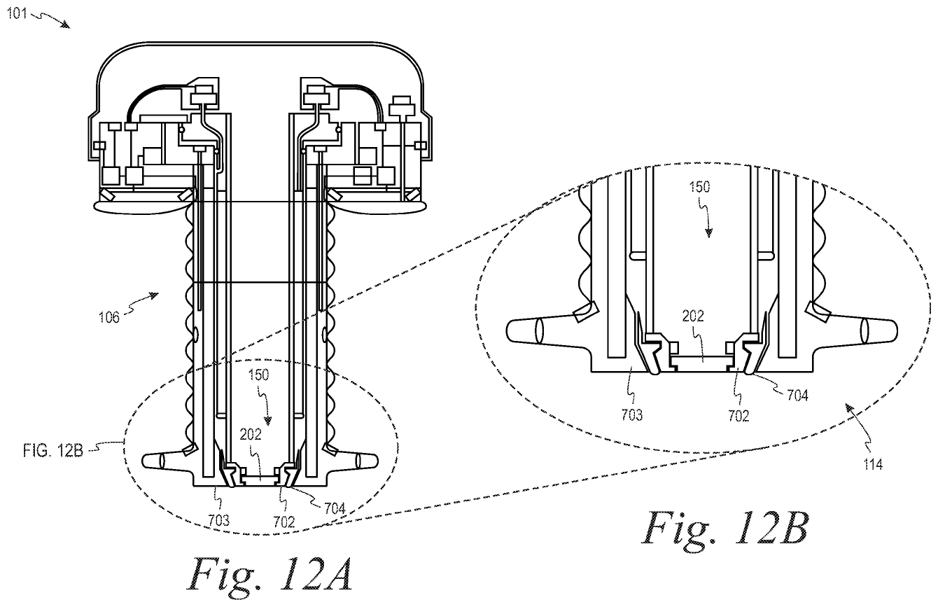

In FIGS. 1A-1C, the proximal port catheter end 105 of the port catheter 104 is fixedly coupled to the distal housing portion 114. This helps provide manufacturing modularity of different port catheter configurations. However, in other examples, the proximal port catheter end 105 of the port tube 116 can be additionally or alternatively coupled to the proximal housing portion 110 and/or the percutaneous body portion 112. In still other examples, the PPIS 101 can omit the port tube 116 (as will be described below with respect to FIG. 12).

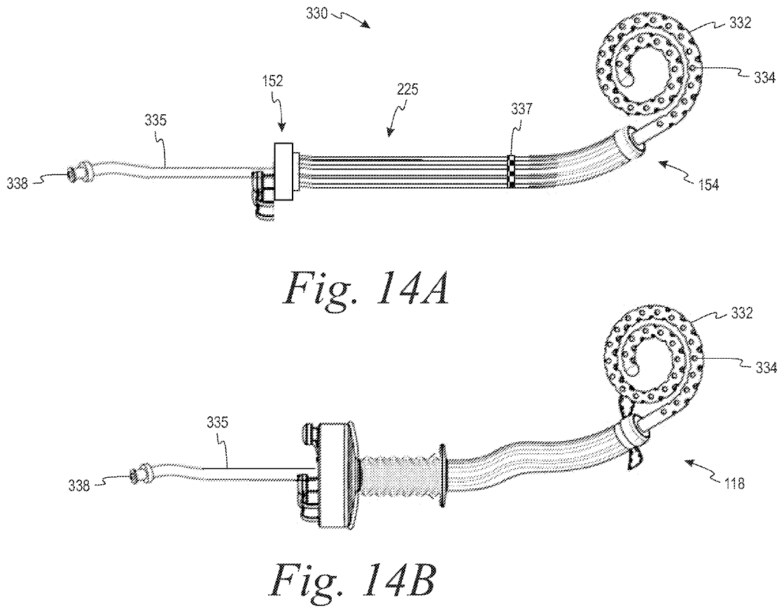

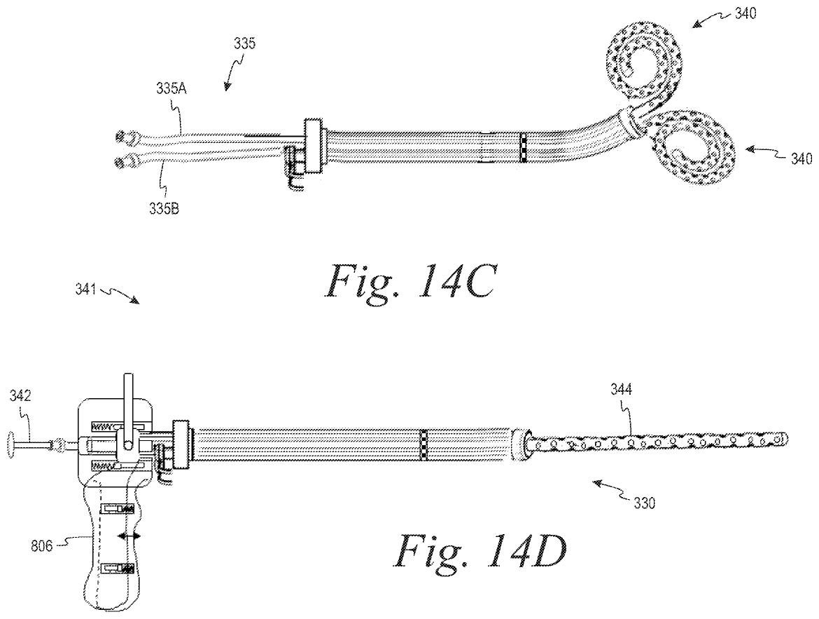

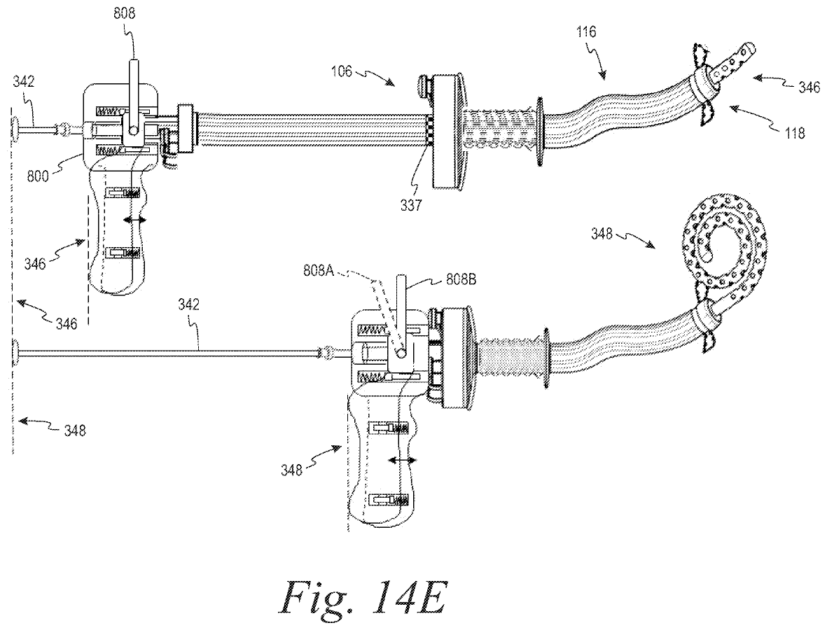

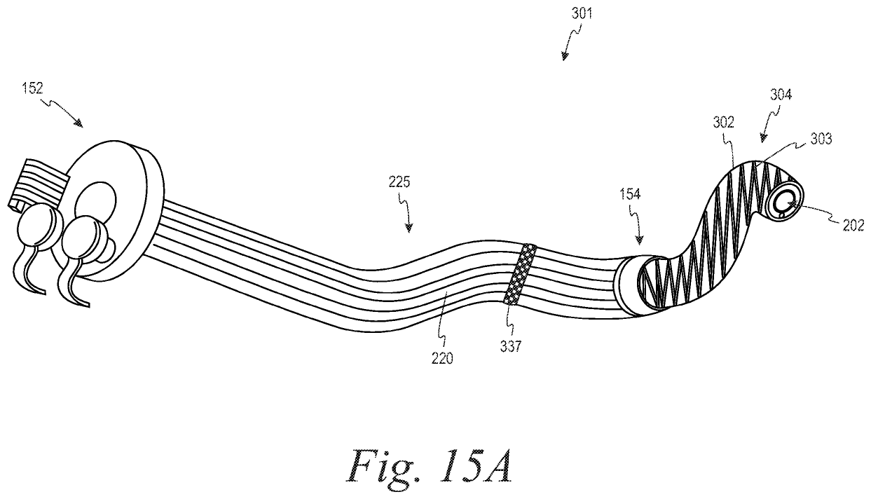

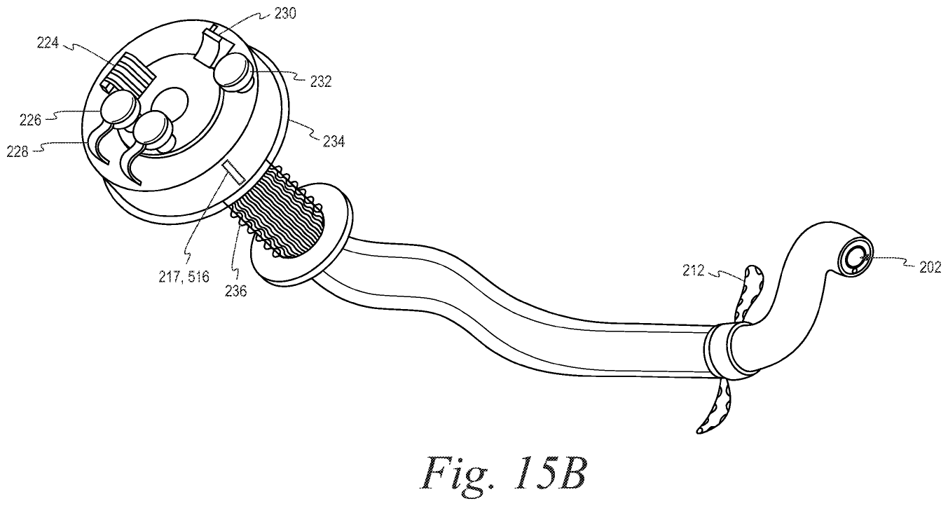

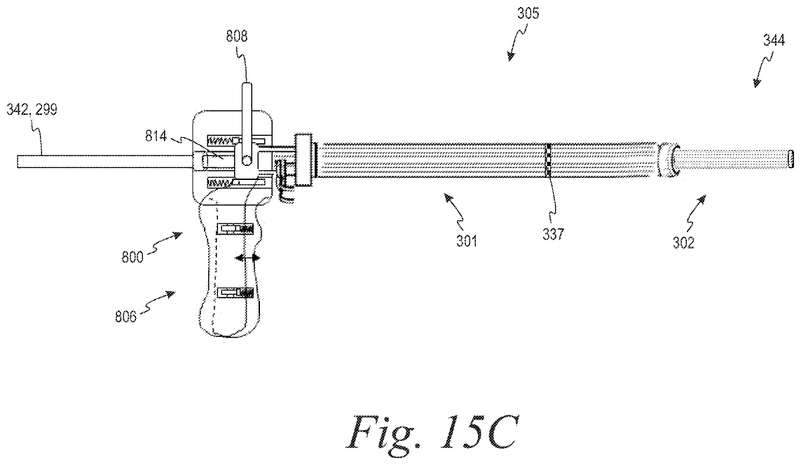

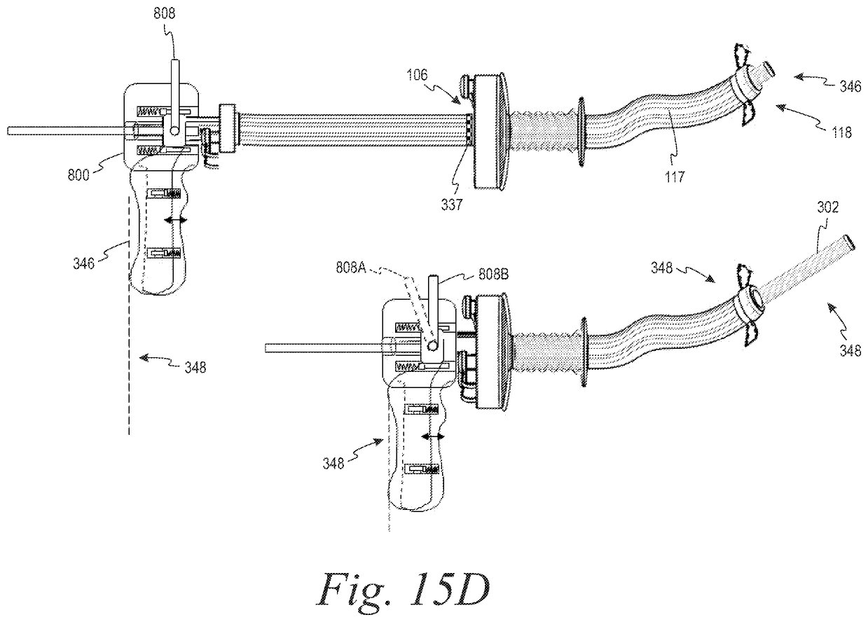

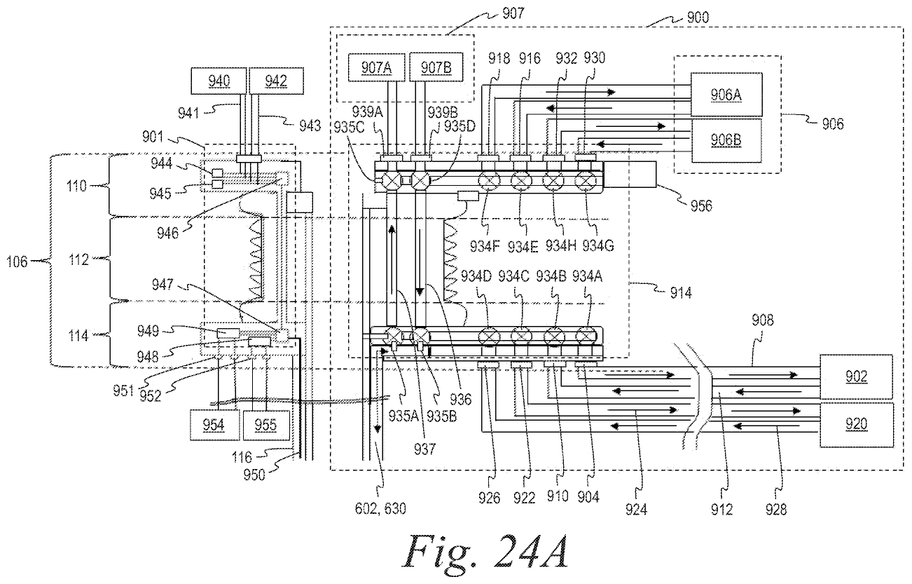

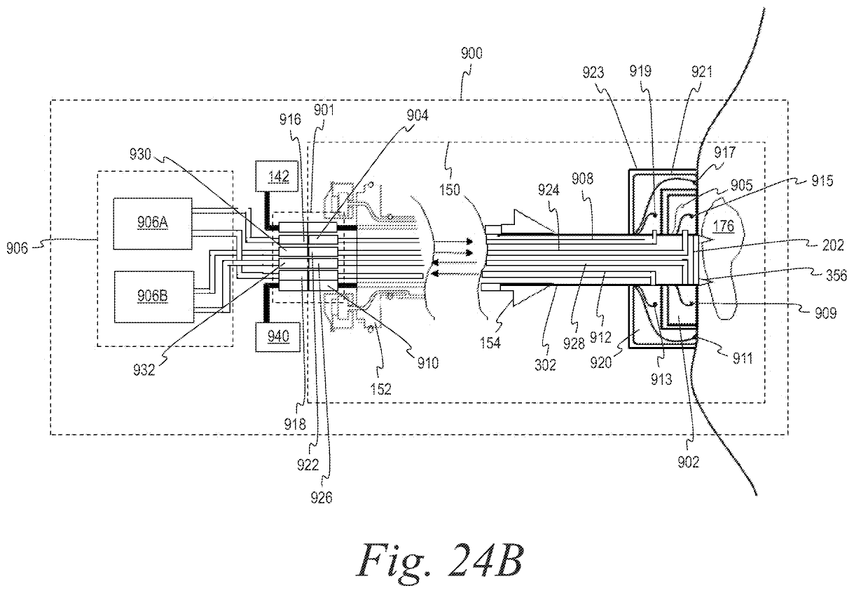

As noted above, FIG. 1A depicts the PPIS 101 in an open configuration. As shown in FIG. 1A, the PPIS lumen 113 is unoccupied. FIG. 1B depicts the PPIS 101 in the closed configuration. As shown in FIG. 1B, the PPIS 101 can further include a plug catheter 150 that is configured to be inserted in the PPIS lumen 113. As described in further detail below, the plug catheter 150 can include and/or receive one or more devices that can facilitate monitoring and/or treating the target tissue 176 and/or other tissues 180, 184 in the body cavity 172. Within examples, the plug catheter 150 is insertable and removable from the PPIS lumen 113. This can provide for the exchange of a multitude of plug catheters 150 configured for specific diagnostic and/or therapeutic purposes deliverable through a single PPIS 101 base design. Several configurations including the fenestrated tube plug catheter FIG. 14A, the extensible plug catheter FIG. 15A, and the thermal bladder and photodynamic therapy catheter FIG. 24B discussed below, illustrate just a few examples.

As described below, the port housing 106, the port catheter 104, and/or the plug catheter 150 can include features that can help to promote recurring aseptic access to the body cavity 172. Additionally, as shown in FIG. 1C, the SDT 100 can include a cap 156 that covers the port housing 106 external to the body 170 (e.g., covering the proximal housing portion 110) when the PPIS 101 is in the closed and capped configuration. This can further help to mitigate infections. Also, as shown in FIG. 1C, the SDT 100 can include an AMT over-bandage 159 that covers the cap 156 providing a water resistant and antimicrobial seal between the patient's body 170 and the PPIS 101. The cap 156 and the AMT over-bandage 159 are described in further detail below.

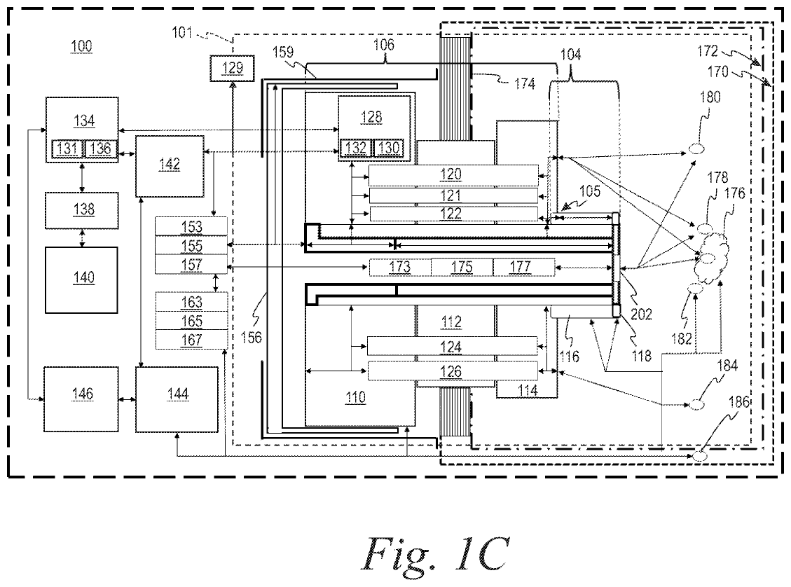

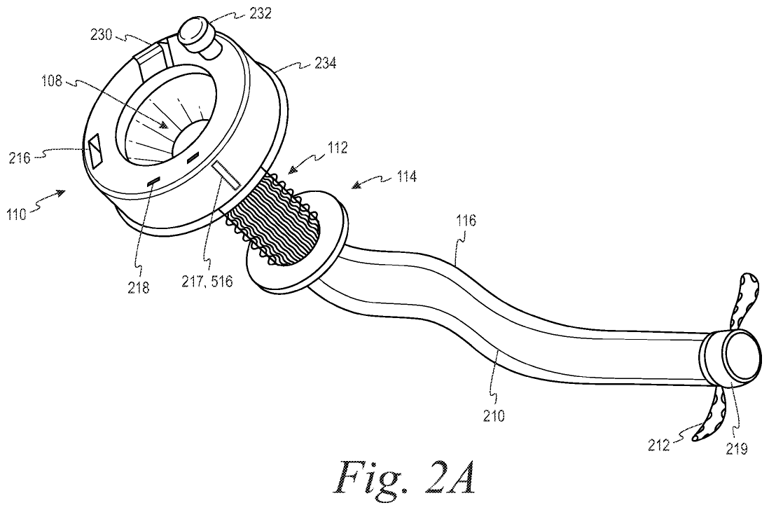

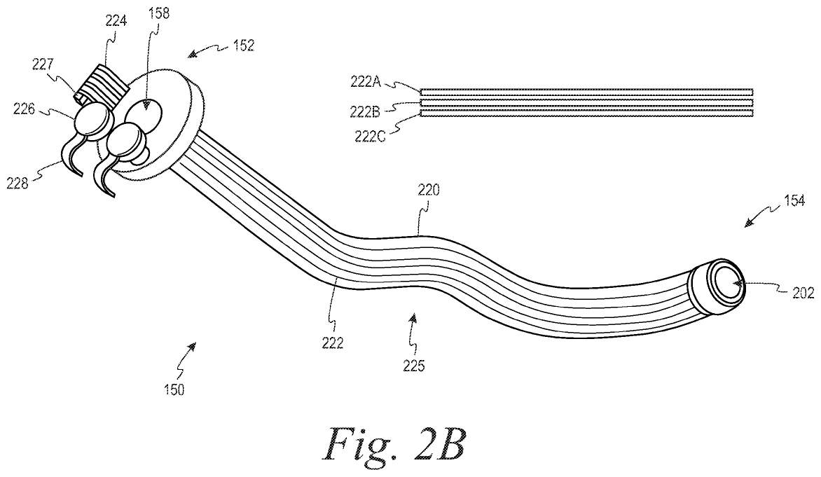

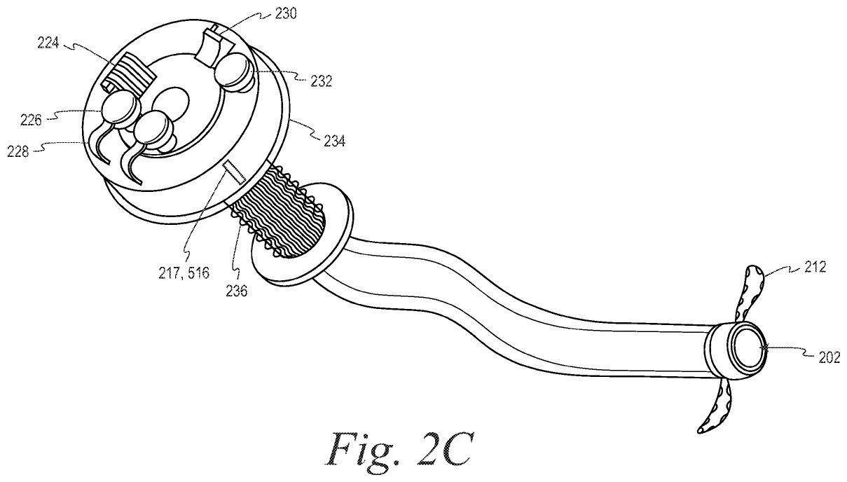

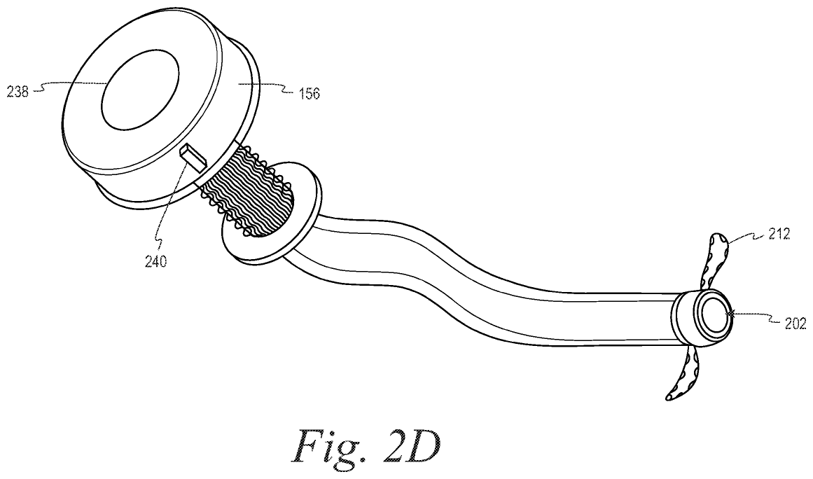

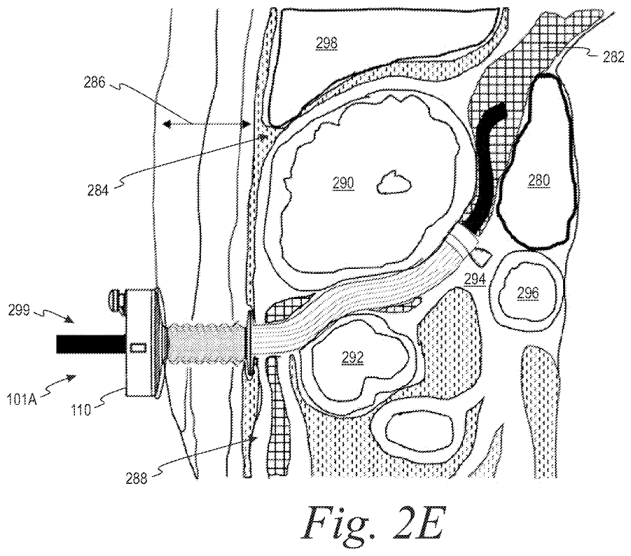

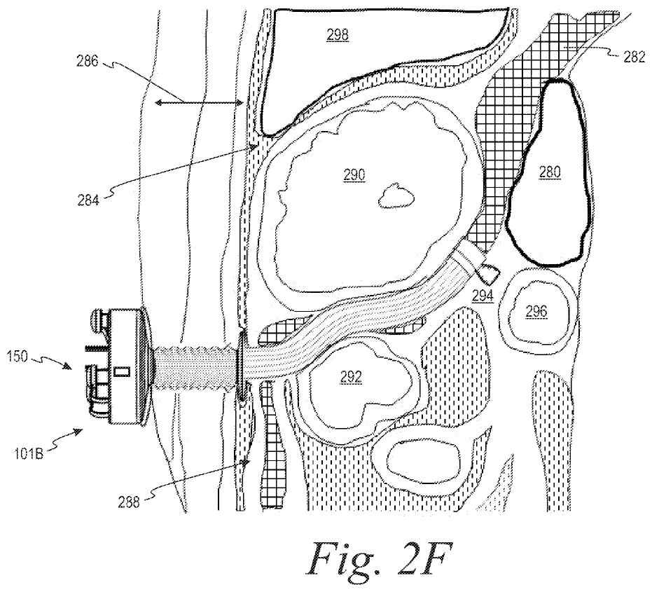

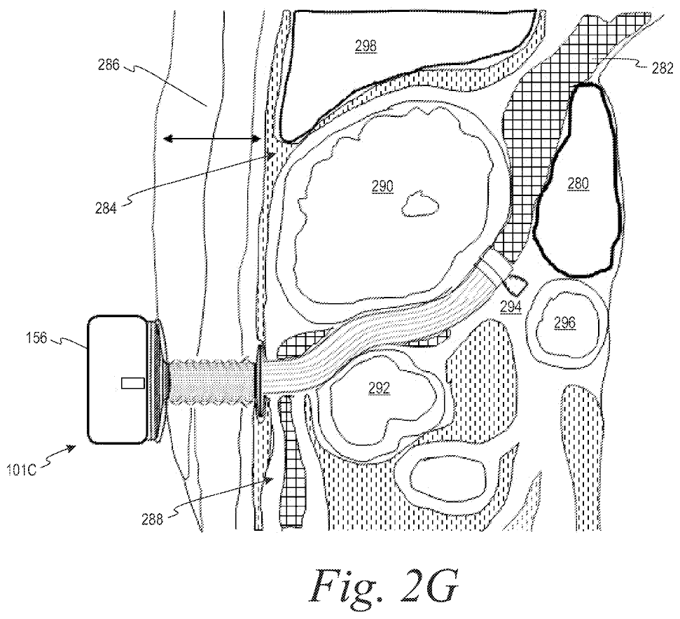

As shown in FIG. 1B, the plug catheter 150 can include a plug tube 225 (shown in FIG. 2B) that defines a plug lumen 158 extending between a proximal plug end 152 and a distal plug end 154. In some examples, the plug lumen 158 can provide a conduit for inserting and/or positioning one or more devices through the PPIS 101 and into the body cavity 172, and/or delivering a therapy through the PPIS 101 and into the body cavity 172. As examples, the plug catheter 150 can be configured to receive, in the plug lumen 158 at least one device selected from a group consisting of: an imaging device, endoscope, an active steering device, an injection device, a biopsy device, a surgical tool, an irrigation device, an insufflation device, a deflation device, an ultrasonic device, an ablation device, a light delivery device, a thermal delivery device, a radiation device, a radiation seed delivery device, a biosensor, and/or any of the devices described below with respect to other systems of the SDT 100.

In the example shown in FIGS. 1B-1C, the plug catheter 150 is configured to be inserted in the PPIS lumen 113 of the PPIS 101 such that a distal plug end 154 extends to the distal port catheter end 118. As described in further detail below, the distal port catheter end 118 can be configured to couple to the target tissue 176 and/or other tissue 180, 182, 184 in the body cavity 172. By positioning the distal plug end 154 at the distal port catheter end 118, the position of the distal plug end 154 and one or more devices coupled to or received in the plug catheter 150 can be more precisely positioned relative to anatomical structures in the body cavity 172. However, as described in further detail below, the distal plug end 154 can extend distally of the distal port catheter end 118 in other examples.

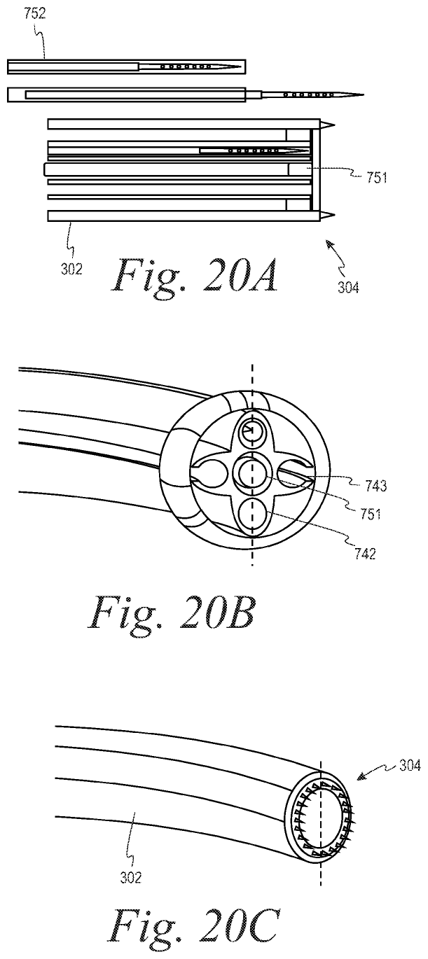

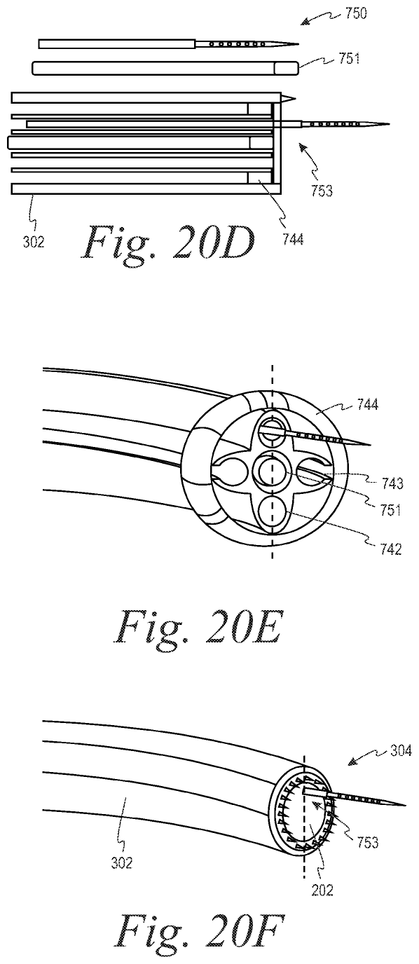

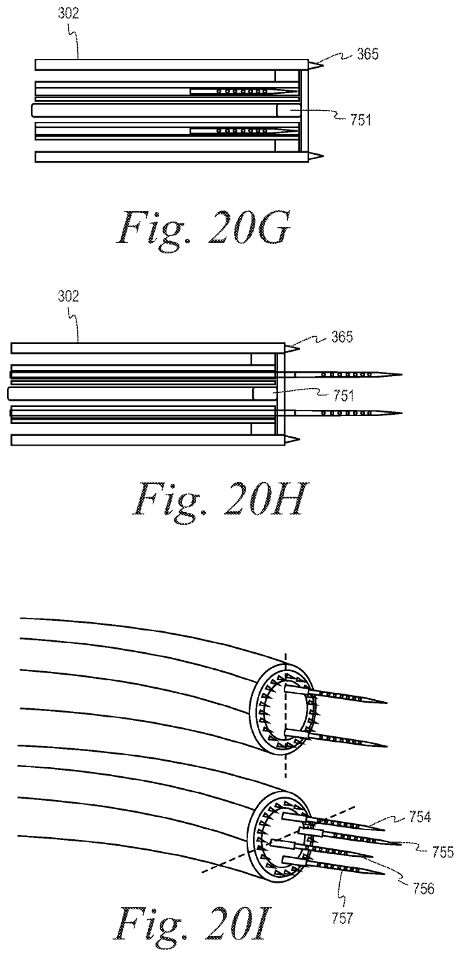

Additionally, in the example shown in FIGS. 1B-1C, the distal plug end 154 of the plug catheter 150 can include a closed window 202 that is configured to seal the plug lumen 158 from the body cavity 172. The window 202 can help to promote aseptic access of the body cavity 172 in some examples. For instance, the closed window 202 can include a self-healing window that is configured to (i) form seal around a device in the plug lumen 158 that pierces through the closed window 202 at a puncture site 753 (shown in FIGS. 20A-20I) and (ii) reseal the closed window 202 at the puncture site 753 responsive to the device ceasing to pierce through the closed window 202. As examples, the closed window 202 of the PPIS 101 can be formed from at least one material selected from a group consisting of: silicone rubber, thermoplastic polyurethane (TPU), thermoplastic elastomer (TPE). Example implementations of the self-healing window 202 are shown and described below with respect to FIGS. 2B-2C and 20A-20I.

In some examples, the closed window 202 of the PPIS 101 is additionally or alternatively configured to allow an imaging device to capture an image through the closed window 202. For instance, the closed window 202 can be formed from at least one optically-transmissive material and/or ultrasonically-transmissive material selected from a group consisting of: silicone rubber, thermoplastic polyurethane (TPU), thermoplastic elastomer (TPE), Ultra-High Molecular Weight Polyethylene (UHMW-PE), Polyether Ether Ketone (PEEK), Polysulfone (PS), Acetal Copolymer (Polyoxymethylene), Polymethyl methacrylate (PMMA), Polypropylene (PP), Polycarbonate (PC), glass and quartz.

In other examples not shown, the distal plug end 154 can include a plurality of closed windows 202, where one or more of the closed windows 202 are self-healing windows and one or more of the closed windows 202 are imaging windows. This can be beneficial in some implementations in which the plug catheter 150 includes at least one imaging device that is configured to capture an image through the imaging window(s) and at least one other device that is configured to puncture through the self-healing window(s).

As shown in FIGS. 1A-1C, the SDT 100 can include the PPIS 101 and one or more additional systems in some examples. As examples, FIGS. 1A-1C show that the SDT 100 can include the PPIS 101 and one or more additional systems selected from a group consisting of: a Continuous Cancer Monitoring system (CCM) 122, 123, 133, 143, 153, 163, 173, a Tumor Microenvironment Management system (TMM) 124, 125, 135, 145, 155, 165, 175, a Cancer Therapeutics Delivery system (CTD) 126, 127, 137, 147, 157, 167, 177, and an Artificial Intelligence System & Software Enterprise (AIS) 128, 134.

The CCM, TMM, and/or CTD can (i) pass through the PPIS 101, (ii) be physically incorporated into the PPIS 101, and/or (iii) reside independently within the PPIS 101. For instance, the CCM, the TMM, and/or the CTD can be coupled to the port housing 106, coupled to the port catheter 104, inserted through the PPIS lumen 113, coupled to the plug catheter 150, and/or inserted through the plug catheter 150. As described in further detail below, the CCM, TMM, and/or CTD can interact and communicate with each another and/or the other systems of the SDT 100, including other external diagnostics therapeutics & data systems 144 through the AIS 134 and a graphical user interface (GUI) 138 to the Healthcare Team 140 as shown in FIGS. 1A-1C.

Accordingly, within examples, the CCM 122, 123, 133, 143, 153, 163, 173 can include real-time and/or periodically sampled diagnostic biosensors, imaging systems, and molecular technologies placed in, on and around, distant to the target tissue 176 and/or within the body 170. The CCM can be used to measure physiological measures, biomarkers, and/or analytes. In some implementations, the CCM can also be used as a feedback system in conjunction with the other subsystems of the PPIS 101 and SDT 100. As an example, an automated spectral analysis and imaging system that independently resides in the plug catheter 150 for extended periods of time where the automated spectral analysis and imaging system utilizes a window 202 of the plug catheter 150 to image the target tissue 176 and is controlled and communicated to through the PPIS 101. Additional aspects and implementations of the CCM are described in further detail below.

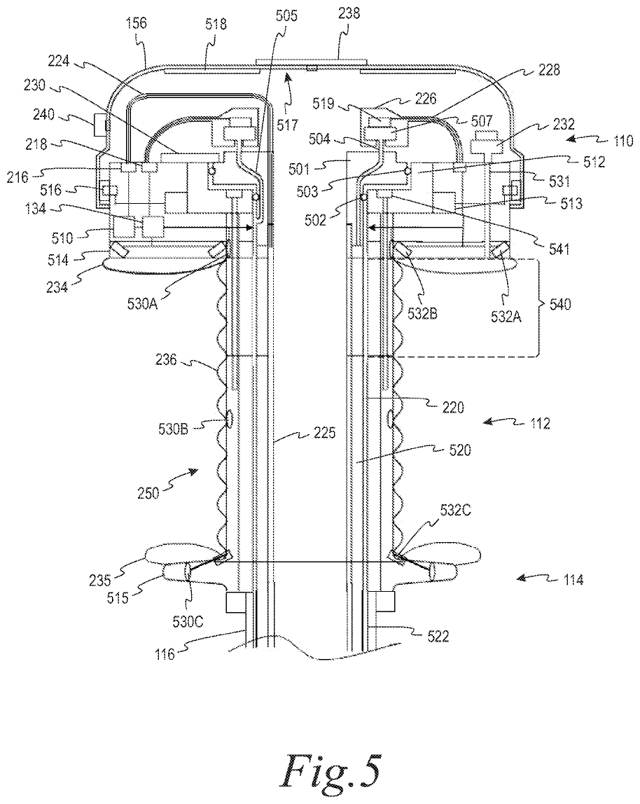

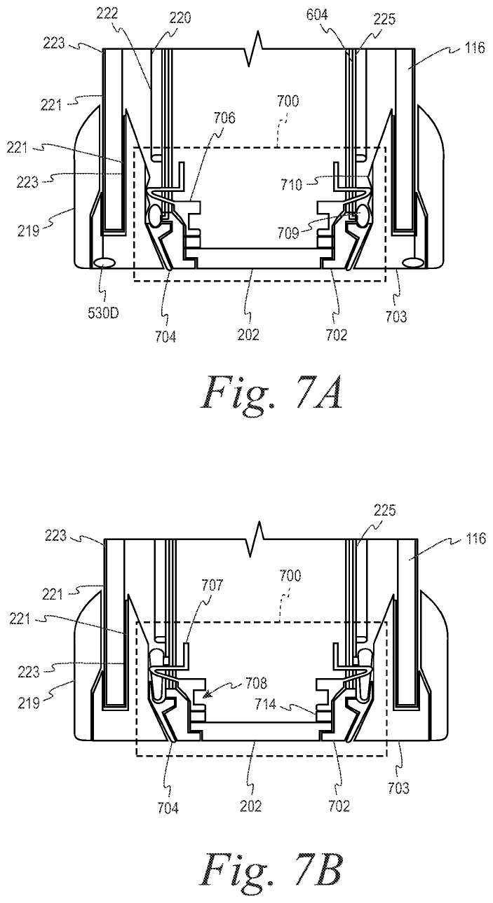

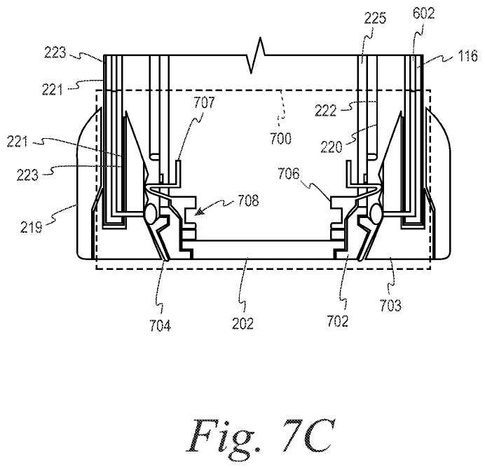

Within examples, the CCM can include one or more sensors (e.g., a sensor 365, 366, 530A-530D, 365, 366 shown in FIGS. 5 and 7A) that are configured to sense one or more biological parameters related to a tissue in the body cavity 172, and the sensor(s) can be configured to access the body cavity 172 via the PPIS 101. For instance, as noted above, the sensor(s) of the CCM can be coupled to the port housing 106, coupled to the port catheter 104, inserted through the PPIS lumen 113, coupled to the plug catheter 150, and/or inserted through the plug catheter 150.

In one example implementation, the sensor of the CCM can include a first sensor coupled to the port housing 106 (e.g., the first sensor can be included in the CCM 122 shown in FIGS. 1A-1C) and a second sensor coupled to the plug catheter 150 (e.g., the second sensor can be included in the CCM 173 shown in FIG. 1C). In another example implementation, the sensor of the CCM can include a first sensor coupled to the port housing 106, a second sensor coupled to the port catheter 104, a third sensor coupled to the plug catheter 150, and/or a fourth sensor inserted through the plug catheter 150. As shown in FIG. 1C, the first sensor, the second sensor, the third sensor, and/or the fourth sensor can be positioned as described above while the plug catheter 150 is positioned in the PPIS lumen 113 and/or the cap 156 is coupled to the port housing 106. In yet another implementation, the sensor of the CCM can include a first sensor coupled to the port housing 106, a second sensor coupled to the port catheter 104, and a third sensor inserted through the port catheter 104.

Within examples, the sensor(s) of the CCM can be configured to be positioned in the target tissue 176, the sensor(s) of the CCM can be configured to be positioned on the target tissue 176, and/or the sensor(s) of the CCM can be configured to be positioned at a location that is spaced away from the target tissue 176 (e.g., at the tissue 180, 182, 184). A benefit of placing multiple CCM in the various locations is the collection of both local and systemic real-time biometric data with significant resolution. This data can be utilized by the AIS and external databases 144 using artificial intelligence algorithms to inform on the status of an individual patient and categories of patients with similar disease state profiles to allow the Healthcare Team to create better and more targeted treatment plans through a single PPIS 101 and SDT 100. More details are discussed below.

As examples, the sensor of the CCM can include at least one sensor selected from a group consisting of: a biosensor, an imaging sensor, and a molecular assay. Additionally, as examples, the biological parameter that can be sensed by the sensor of the CCM can include at least one biological parameter selected from a group consisting of: a physiological measure, a biomarker, and an analyte. In some examples, the biological parameter can include at least one biological parameter selected from a group consisting of: a temperature, an interstitial fluid pressure, a heart rate, a respiration rate, a rate of blood flow, an oxygen saturation level, a pH, an amount of glucose, an amount of lactate, an amount of carbohydrate antigen 19-9 (CA-19-9), an amount of carcinoembryonic antigen (CEA), an amount of cancer antigen 125 (CA125), an amount of CA242, an amount of vascular endothelial growth factor (VEGF), and an amount of PD-1/PD-L1. In some examples, the biological parameter can additionally or alternatively include at least one parameter selected from a group including: a temperature, a heart rate, a blood pressure, PO2, pH, lactate, cytokines, liver enzymes—including alanine transaminase (ALT) and aspartate transaminase (AST), alkaline phosphatase (ALP), gamma-glutamyl transferase (GGT), serum bilirubin, prothrombin time (PT), the international normalized ratio (INR), total protein and albumin.; kidney—Blood urea nitrogen (BUN), serum creatinine, molecule-1 (KIM-1), β2-microglobulin (B2M), cystatin C, clusterin, and trefoil factor-3 (TFF-3), IL-18 (interleukin-18), NGAL (neutrophil gelatinase-associated lipocalin), Netrin-1, liver type fatty acid binding protein (L-FABP), urinary exosomes, and TIMP2 (insulin-like growth factor-binding protein 7)/IGFBP7 (insulin-like growth factor binding protein 7); brain—pro-inflammatory cytokines (Interleukin-1, Tumor Necrosis Factor, Interleukin-6), and the anti-inflammatory cytokines (IL-4, Interleukin-10, and TGF-alpha and beta).

In some examples, the sensor(s) of the CCM is configured to measure at least one of a bioavailability parameter, a pharmacokinetics parameter, and/or a pharmacodynamics parameter of a drug therapy. In some examples, the sensor(s) of the CCM is additionally or alternatively configured to measure a drug accumulation and intravenous drug accumulation of the tissue, where a ratio of drug accumulation divided by intravenous drug accumulation determines an absolute bioavailability. In some examples, the sensor(s) of the CCM is additionally or alternatively configured to measure the pharmacokinetics parameter of the drug therapy, where the pharmacokinetics parameter includes at least one drug parameter selected from a group consisting of: an absorption metric, a distribution metric, a metabolism metric, and an excretion metric. In some examples, the sensor(s) of the CCM is additionally or alternatively configured to measure the pharmacokinetics parameter of the drug therapy, where the pharmacokinetics parameter includes at least one drug parameter selected from a group including: a biochemical effect, a physiologic effect, and a molecular effect.

Within examples, the TTM 124, 125, 135, 145, 155, 165, 175 can include thermal, O₂, pH, and/or Interstitial Fluid Pressure (IFP) measurement biosensors as examples and/or corresponding environmental stimulation hardware and software that is configured to apply one or more stimuli to an environment in which a tissue 176, 178, 180, 182, 184, 186 is located in the body cavity 172 or body 170. The TMM can be configured in multiple ways as part of the PPIS 101 and SDT 100 systems. For instance, in some implementations, the TMM can provide continuous biosensor measurement, environmental stimulation and feedback control that can be used to maintain specifically targeted tumor and tissue microenvironmental parameters through combinations of environmental stimulation devices.

The pancreatic tumor has high IFP, low oxygen tissue concentration and low pH. As per example, the temperature of a solid tumor can be elevated and maintained by the TMM at a specific or modulated set of temperatures in combination with drug therapy to enhanced the pharmacokinetics and pharmacodynamics of the drug, lower and maintain the IFP of a solid tumor, and lower and/or increase and/or maintain the surrounding tissues at a specific or modulated set of IFPs before, during, and after cancer treatment. This is particularly beneficial for directly treating solid tumors such as pancreatic tumors which have elevated IFP levels which significantly diminishes systemic drug delivery efficiency. In another example, the O₂ tissue concentration of a solid tumor can be increased and/or decreased and maintained by the TMM at a specific or modulated O₂ tissue concentration to enhance a treatment. This is particularly beneficial for directly treating solid tumors such as pancreatic tumors which have low O₂ levels. In another example, the pH of a solid tumor can be increased and/or decreased and maintained by the TMM at a specific or modulated pH tissue to enhance a treatment. This is particularly beneficial for directly treating solid tumors such as pancreatic tumors which have low pH levels.

In some examples in which the TMM is configured to monitor oxygen, the TMM can include PaO₂ and PO₂ sensors that are configured with an electrochemical electrode system or an optical fiber system. The TMM can be further used in combination with O₂ delivery systems for the addition of O₂ to the body 170 through various methods including direct O₂ to the target tissue 176, increased respiratory O₂ to the patient, hyperbaric O₂ therapy and/or combinations thereof.

In some examples, the TMM and/or the CCM can additionally or alternatively monitor pH, and the SDT 100 can use measured pH information as an indication of how the body/tumor is responding to treatment. Different treatment modalities will cause changes in pH (increases and decreases) of the targeted and surrounding tissues. Changes in pH can also be an indicator of cancerous tissue and the pH can indicate the presence or absence of cancerous tissue over time. The magnitude and duration of the pH changes are another vector of information for the AI system. The pH sensor can include several examples including electrochemical electrode systems and/or optical methods with immobilized indicator dyes or fluorophores. As examples, the pH sensors of the TMM and/or CCM can be an electrochemical electrode system or an optical fiber system with immobilized indicator dyes or fluorophores and combinations thereof, where the pH sensors are powered and communicate through the PPIS 101.

In some examples, the TMM and/or the CCM can additionally or alternatively monitor temperature, and the SDT 100 can use measured temperature information as an indication of an infection or unwanted adverse events to a particular treatment. In some implementations, the TMM and/or the CCM can include one or more temperature probes that are configured to monitor tissue heating and maintain/adjust optical fluences during photodynamic therapy (PDT) treatment protocols to ensure the tissue stays within prescribed treatment ranges to prevent thermal or unwanted photobiological damage. The CCM can also monitor and maintain/adjust thermal treatments to know the extent of tissue volume damage that the treatment produced. The TMM can also be used to monitor the progression of disease solely or in combination with other diagnostics to determine the success or failure of various treatment protocols. Within examples, the TMM and/or the CCM can include one or more thermocouples, thermistors, and/or optical temperature sensors, which are powered through and communicate through the PPIS 101 to monitor temperature.

Accordingly, within examples, the TMM can include an environmental stimulator (e.g., an environmental stimulator shown in FIGS. 24A-24B) that is configured to apply one or more stimuli to an environment in which a target tissue 176 is located in the body cavity 172, and the environmental stimulator can be configured to access the body cavity 172 via the PPIS 101. As noted above, the environmental stimulator of the TMM can be coupled to the port housing 106, coupled to the port catheter 104, inserted through the PPIS lumen 113 of the PPIS 101, coupled to the plug catheter 150, and/or inserted through the plug catheter 150.

In one implementation, the environmental stimulator can include a first environmental stimulator (e.g., the first environmental stimulator can be included in the TMM 124) coupled to the port housing 106 and a second environmental stimulator coupled to the plug catheter 150 (e.g., the second environmental stimulator can be included in the TMM 175). In another implementation, the environmental stimulator can include a first environmental stimulator coupled to the port housing 106, a second environmental stimulator coupled to the port catheter 104, a third environmental stimulator coupled to the plug catheter 150, and/or a fourth environmental stimulator inserted through the plug catheter 150. In yet another example implementation, the environmental stimulator can include a first environmental stimulator coupled to the port housing 106, a second environmental stimulator coupled to the port catheter 104, and a third environmental stimulator inserted through the port catheter 104.

Within examples, the environmental stimulator(s) of the TMM can be configured to be positioned in the target tissue 176, the environmental stimulator(s) of the TMM can configured to be positioned on the target tissue 176, and/or the environmental stimulator(s) of the TMM can configured to be positioned at a location that is spaced away from the target tissue 176 (e.g., at the tissue 180, 182, 184). A benefit of the TMM at the various locations is to provide local management of the tumor microenvironment and systemic environmental management of the body simultaneously over a short and/or extended period of time through a single PPIS 101 and SDT 100. More details are discussed below.

As examples, the environmental stimulator of the TMM system can include at least one environmental stimulator selected from a group including: a liquid delivery system, a gas delivery system, a vacuum system, a thermal energy delivery system, a light delivery system, and a radiation delivery system. Additionally, as examples, the environmental stimulator of the TMM system can additionally or alternatively include at least one environmental stimulator selected from a group including: a resistive heater, an electromagnetic energy delivery device, an ultrasonic energy delivery device, a radio frequency (RF) energy delivery device, a microwave energy delivery device, a fluidic heat exchanger system, a chemical thermal heat exchanger system configured to produce a chemical exothermic reaction and/or an endothermic reaction, and a thermoelectric device.

In an implementation in which the environmental stimulator includes the electromagnetic energy delivery device, the electromagnetic energy delivery device can be configured to generate electromagnetic energy at frequency that corresponds to an absorption frequency of the target tissue. In an implementation in which the environmental stimulator includes the ultrasonic energy delivery device, the ultrasonic energy delivery device can be configured to generate ultrasonic energy at frequency that corresponds to an absorption frequency of the target tissue. In an implementation in which the environmental stimulator includes the electromagnetic energy delivery device, the electromagnetic energy delivery device can be configured to generate electromagnetic energy at frequency that corresponds to an absorption frequency of a particle absorber positioned in the environment of the target tissue, where the particle absorber is positioned at at least one position selected from a group including: a position adjacent to the target tissue, a position on the target tissue, and a position in the target tissue. In an implementation in which the environmental stimulator includes the ultrasonic energy delivery device, the ultrasonic energy delivery device can be configured to generate ultrasonic energy at frequency that corresponds to an absorption frequency of a particle absorber positioned in the environment of the target tissue, where the particle absorber is positioned at at least one position selected from a group including: a position adjacent to the target tissue, a position on the target tissue, and a position in the target tissue.

Within examples, the CTD can include a therapeutic treatment device that is configured to deliver a therapeutic treatment to a tissue in the body cavity 172. The therapeutic treatment device can access the body cavity 172 via the PPIS 101. For instance, the therapeutic treatment device can be coupled to the port housing 106, coupled to the port catheter 104, and/or inserted through the outer lumen 111 of the port catheter 104 (e.g., through the PPIS lumen 113). As examples, the CTD can be configured such that the therapeutic treatment includes at least one treatment modality selected from a group consisting of: drug therapy, thermal therapy, photodynamic therapy, laser ablation, radiofrequency ablation (RFA), microwave ablation (MWA), high-intensity focused ultrasound (HIFU), irreversible electroporation (IRE), and light-activated nanoparticles ablation therapy. In other examples, the therapeutic treatment device can additionally or alternatively include a thermal circulatory bladder system and/or a fluid manifold as described in further detail below with respect to FIGS. 24A and 24B.

The AIS 128, 134 can be implemented using hardware, software, and/or firmware. For example, the AIS 128, 134 can include one or more processors and a non-transitory computer-readable medium (e.g., volatile and/or non-volatile memory) that stores machine language instructions or other executable instructions. The instructions, when executed by the one or more processors, cause the AIS 128, 134 to carry out various operations described herein. The AIS 128, 134 can, thus, can receive data (including data indicated by CCM, the TMM, the CTD, the GUI 138, the Healthcare Team 140, and/or the databases 146) and store the data in memory as well.

The processor(s) and/or the non-transitory computer-readable medium can be implemented in any number of physical devices/machines. For example, the AIS 128, 134 can include one or more shared or dedicated general purpose computer systems/servers. Accordingly, principles and advantages of distributed processing, such as redundancy, replication, and the like, also can be implemented, as desired, to increase the robustness and performance of the devices and systems of the AIS 128, 134.

The physical devices/machines can be implemented by the preparation of integrated circuits or by interconnecting an appropriate network of conventional component circuits, as is appreciated by those skilled in the electrical art(s). The physical devices/machines, for example, may include field programmable gate arrays (FPGA's), application-specific integrated circuits (ASIC's), digital signal processors (DSP's), etc. The physical devices/machines may reside on a wired or wireless network, e.g., LAN, WAN, Internet, cloud, near-field communications, etc., to communicate with each other and/or other systems (e.g., the PPIS 101, the CCM 122, 123, 133, 143, 153, 163, 173, the TMM 124, 125, 135, 145, 155, 165, 175, and/or the CTD 126, 127, 137, 147, 157, 167, 177) using, for example, Internet/web resources.

As shown in FIGS. 1A-1C, in some examples, the AIS 128, 134 can include one or more regular processors (130, 131) and/or one or more neuromorphic processors (132, 136) configured to communicate with the external and internal hardware, software, firmware, and interfaces 120 contained within the systems of the SDT 100. A benefit of using regular and neuromorphic processors in combination is that it can allow for the distribution of basic system management and control via regular processors versus the significant computational capacity for artificial algorithm analysis via neuromorphic processors, and their subsequent control and control of feedback systems in real-time for the CCM, TMM, and CTD systems through a single PPIS 101 and SDT 100.

The SDT 100 can also include one or more external Databases 146, GUIs 138 and their interfaces with one or more treatment plans 142 that are generated with additional external diagnostics, therapeutics and data 144 through the databases 146 and the AIS system 134 and 128 of the PPIS 101. The AIS 128, 134 can be configured to use both the regular processors 130, 131 and neuromorphic processors 132, 136 to monitor, analyze and control the CCM, TMM and CTD subsystems and the AMT 121 through the PPIS 101 and SDT 100.

In some implementations, the AIS 128, 134 can use the gathered real-time data from the CCM and/or the TMM alone or in combination with other externally gathered data 144 and databases 146. In some implementations, the AIS 128, 134 can use algorithms including both Recurrent (Temporal) and/or Convolutional (Spatial) Neural Networks (RNN and CNN) and the neuromorphic processors 132, 136 can be used to analyze the data 144, 146. These systems can be semi-permanently placed (e.g., weeks, months, years) to continuously and/or periodically treat and gather biometric data related to the state of an individual's disease and health status and combined with any other data of the individual's medical record usually in the form of an electronic medical record (EMR) within the databases 146. This data can then be downloaded and aggregated over time and utilized by specialized artificial intelligence (AI) learning algorithms using GUI 138 with spatiotemporal mapping to visualize the evolution of data for different combinations of CCM, TMM, and CTD with space and time and the correlations between them to inform on a patient's disease state regarding progression or remission utilizing the neuromorphic processors 132, 136 of the PPIS 101 and SDT 100. The software allows for the aggregation of others with similar disease states and allows for the Healthcare Team 140 to prognosticate on treatment plan 142 and protocol algorithms to enhance positive disease state treatment outcomes of the various treatment protocols. The AIS 128, 134 is configured to integrate into mobile applications for the Healthcare Team 140 and another for the Patient which including functions such as physical parameter feedback, biomarker feedback, toxicity and infection monitoring and alerts, system status and alerts, reports, patient feedback, scheduling, etc.

In some examples, data collected from individual patients can also be collected and run on external neuromorphic processors 136 through additional RNN and CNN coupled to the neuromorphic processors 132 to improve overall RNN and CNN combination feedback for various categories of disease states where the initial patient inputs and subsequent treatment outcomes inform on the overall efficacy and toxicity profiles of the various diagnostics and therapeutics. This system and method also allows for the variation (elimination or addition) of new and future diagnostics and therapeutics as they are developed and implemented as previous data within the databases 146 can be applicable as long as the various conditions are well known and/or controlled.

Within examples, the CCM, TMM, CTD, AIS, and/or AMT systems powered and communicated through the PPIS 101 and SDT 100 can be tethered or untethered to the PPIS 101 where the PPIS 101 is configured to be the treatment delivery and communication hub for the various CCM, TMM, CTD and AIS devices and systems. The advantage of this hub approach is the sharing of device hardware, power and communication resources and the shortening of power and communication distances between devices delivered through the PPIS 101

As described above, the processor(s) 130, 131, 132, 136 can (i) be communicatively coupled to the CMM, the TMM, and/or the CTD, (ii) receive information from the CMM, the TMM, and/or the CTD, and (iii) responsively take an action based on the received information.

In one example, the processor(s) 130, 131, 132, 136 can be in communication with the sensor of the CCM system, and the processor 130 is configured to: (i) receive, from the sensor of the CCM system, a signal indicative of the biological parameter, (ii) make a determination, based on the signal, that an alert condition has occurred, and (iii) responsive to the determination, cause an output device 129 to generate an alert. For instance, the output device can be configured to generate the alert as a visual alert, an auditory alert, and/or a tactile alert. As examples, the output device can be at least one device selected from a group consisting of: a mobile phone, a tablet computer, and a personal computer.

In some implementations, the processor(s) 130, 131, 132, 136 can be additionally or alternatively configured to communicate the alert as an electronic message communicated over a communications network. In one implementation, the electronic message can be a short message service (SMS) protocol message and/or an e-mail. The communication network can include at least one communication connection selected from a group including: a Bluetooth connection, a wireless local-area network (WLAN) connection, a cellular connection, a radio frequency connection, and an infrared communication connection. The system where the communication network includes at least one communication connection selected from a group consisting of: a Bluetooth connection, a wireless local-area network (WLAN) connection, a cellular connection, a radio frequency connection, and an infrared communication connection.

In another example, the processor(s) 130, 131, 132, 136 can be in communication with the sensor of the CCM system and the at least one therapeutic treatment device. In this example, the processor is configured to perform a feedback operation including: (i) receiving, from the sensor of the CCM system, a signal indicative of the biological parameter, (ii) determining, based on the signal received from the CCM system, a treatment parameter relating to operation of at least one therapeutic treatment device, and (iii) causing at least one therapeutic treatment device to deliver, in accordance with the treatment parameter, the therapeutic treatment to the tissue. In some implementations, determining the treatment parameter can include determining the treatment parameter such that a value of the biological parameter is maintained within a predetermined range of values responsive to the at least one therapeutic treatment device delivering the therapeutic treatment to the tissue in accordance with the treatment parameter. Also, within examples, the processor 130, 131, 132, 136 can be configured to perform the feedback operation on a continuous basis and on a periodic basis.

In an implementation of this example, the processor(s) 132, 136 can include the neuromorphic processor 132, and the neuromorphic processor 132 can be in the port housing 106. In another implementation of this example, the processor(s) 132, 136 can include the neuromorphic processor 132 in communication with the neuromorphic processor 136, where the neuromorphic processor 132 is in the port housing 106 and the neuromorphic processor 136 is external to the port housing 106. This implementation can provide for the creation of adaptable autonomous feedback and control systems. As artificial algorithms of the AIS learn over time, they can modify and enhance the ongoing treatment parameters of the CCM, TMM and CTD in real-time. This can be particularly important for the monitoring and control of toxicity and/or treatment efficacy and to improve overall treatment for the individual patient as discussed below.

In an example operation, after a patient has been diagnosed with a particular form of cancer, for example Pancreatic Cancer, the patient's healthcare team 140 can create a treatment plan 142 through the GUI 138 of the AIS software determining, for example, the initial types, numbers, and diagnostic and the neoadjuvant, primary and adjuvant therapeutic protocols for the PPIS 101, CCM (122,123, 133, 143, 153, 163), TMM (124, 125, 135, 145, 155, 165) and CTD (126, 127, 137, 147, 157, 167) as well as external Diagnostics and Therapeutics and Data 144 to the PPIS 101 to be implemented utilizing the SDT 100. Real-time feedback, control and data can be shared between the PPIS 101 and the external Diagnostics, Therapeutics and external Data 144. The treatment plan 142 can be supported by the GUI 138 interface, the AIS & Software Enterprise 134, and the data within the databases 146.

The surgeon can implant one or more PPIS 101, CCM (122,123, 133, 143, 153, 163), TMM (124, 125, 135, 145, 155, 165) and CTD (126, 127, 137, 147, 157, 167) and AIS 128 subsystems into the body cavity 172 and/or body 170 of the patient. Multiple subsystems can be placed in, on, adjacent to, distant to the target tissues, and/or within the body 170, 172, 174, 176, 178, 180, 182, 184, 186. Several sites can be treated with separate PPIS if the cancerous tissue has metastasized to one or more body cavities of the body. Within examples, the SDT 100 can be configured to treat bladder cancer, breast cancer, colon cancer, kidney cancer, lung cancer, ovarian cancer, pancreatic cancer, prostate cancer, rectal cancer, stomach cancer, thyroid cancer, and uterine cancer.

The initial system and treatment parameters can then be initiated by the Healthcare Team 140. The CCM TMM and AIS of the PPIS 101 and SDT 100 help to measure and maintain/modulate the tumor microenvironment and provide delivery and access of the CTD and the delivery of the treatment combinations through the system hardware, firmware and software interfaces 120.

Feedback from before, during, and after the initial treatment is monitored by the CCM and maintained/modulated by the TMM. This data can be automatically and continuously entered through a hardwire interface and/or a wireless interface. The AI enterprise software 134 can be enabled, which analyzes the data using RNN and CNN software algorithms to begin to determine whether the initial treatment parameters are having a beneficial, neutral, or a deleterious effect. In addition, the CCM can initially monitor toxicity (minutes, hours, days, weeks) but as time goes forward, it can also monitor the therapeutic effect (hours, days, weeks, months).

In some implementations, the SDT 100 can be a continuous feedback system. For instance, the algorithms can initially be fixed but over time can be become adaptable through AI algorithms to allow for the Healthcare Team 140 to fine tune the treatment parameters of the treatment plan 142 to obtain the most beneficial treatment combinations for the individual patient based on their specific response to the on-going treatment. As the AI adaptable algorithms become more sophisticated, the treatment protocols can be automated for autonomous TMM and CTD delivery. Depending on the cancer being treated and the number of sites required to be treated, various combinations of the PPIS 101 shown in FIGS. 1A-1C and CMM, TMM, CTD and AIS subsystems can be grouped in specific combinations and quantities with the example advantages of being more manageable in size, and/or closer to the targeted tissue if there are multiple cancer sites and/or once a patient goes into remission. Specific subsystem combinations can be grouped for short term or sequenced removal while leaving others such as a smaller PPIS and CCM combination to remain over a longer period of time to monitor the patient. Additional combinations can be added or deleted depending on the treatment protocol requirements of the individual patient determined by the Healthcare Team 140.

Referring now to FIG. 2A, a perspective view of an implementation of the PPIS 101 in the open configuration is shown according to an example. As shown in FIG. 2A, the PPIS 101 includes the port housing 106 and the port catheter 104 as described above. Accordingly, the port housing 106 includes the proximal housing portion 110, the distal housing portion 114, and the percutaneous body portion 112 extending between the proximal housing portion 110 and the distal housing portion 114. Additionally, the port housing 106 includes the port lumen 108 extending entirely through the proximal housing portion 110, the percutaneous body portion 112, and the distal housing portion 114. The port catheter 104 includes the port tube 116 that defines the outer lumen 111 (shown in FIG. 1A), and the port lumen 108 of the port housing 106 and the outer lumen 111 of the port catheter 104 define the PPIS lumen 113. Additionally, as shown in FIG. 2A, the distal port catheter end 118 of the port catheter 104 extends distally of the distal housing portion 114.

As shown in in FIG. 2A and FIG. 5, the proximal housing portion 110 of the PPIS 101 can include a proximal flange 514 that extends outwardly from the percutaneous body portion 112, and the distal housing portion 114 can include a distal flange 515 that extends outwardly from the percutaneous body portion 112. As shown in FIGS. 2E-2G, the proximal flange 514 and/or the distal flange 515 can help to retain the port housing 106 at an implant site in the wall 174 (e.g., an abdominal wall 286) of the body 170.

In the example shown in FIG. 2A, the percutaneous body portion 112 has a circular shape. This can help to reduce (or minimize) the cross sectional area through the body wall. In other examples, the percutaneous body portion 112 can have a cross-sectional shape that is selected from among a group consisting of: a circle shape, an oval shape, a polygonal shape, and a nonpolygonal shape.

As shown in FIGS. 2A, 2C, 3A, 3B, and 5, the proximal housing portion 110 can also include an inflatable bumper balloon 234 located on a distal side of the proximal flange 514. Accordingly, when the port housing 106 is positioned with a body wall 174 at the percutaneous body portion 112, the inflatable bumper balloon 234 can be inflated through an inflation port 232 and an inflation tube 531 coupled to a fluid source to adjust a distance between the proximal housing portion 110 and the body wall 174 (e.g., the inflatable bumper balloon 234 can fill the gap between the proximal housing portion 110 and the body wall 174, which helps to secure and mitigate distal movement of the port housing 106 to and through the body wall 174).

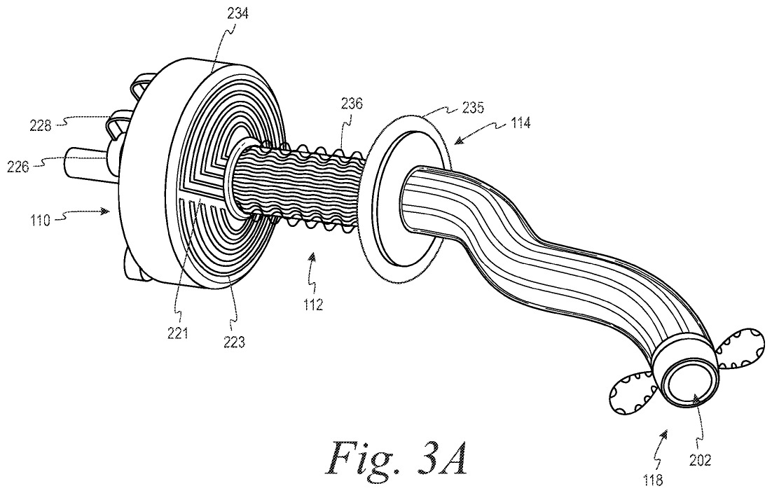

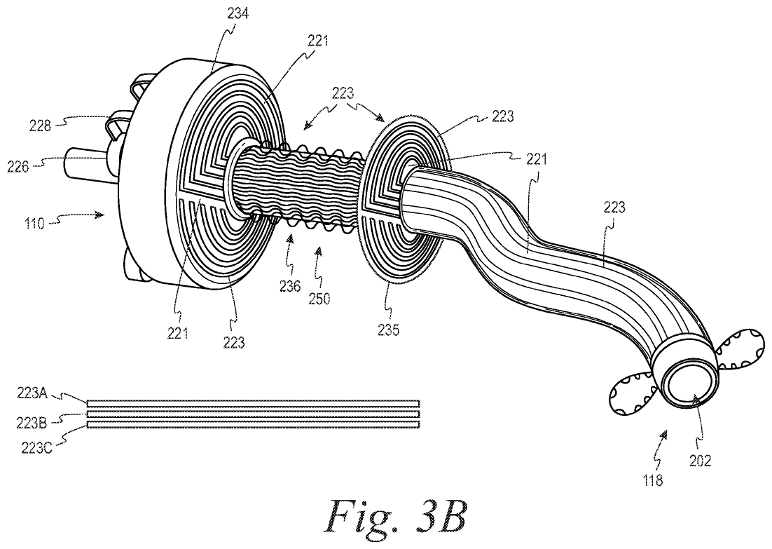

As shown in in FIGS. 3A-3B and 5, the distal housing portion 114 can also include an inflatable bumper balloon 235 located on a proximal side of the distal flange 515. Accordingly, when the port housing 106 is positioned with a body wall 174 at the percutaneous body portion 112, the inflatable bumper balloon 235 can be inflated through the inflation port 232 and the inflation tube 531 coupled to the fluid source to adjust a distance between the distal housing portion 114 and the body wall 174 (e.g., the inflatable bumper balloon 235 can fill the gap between the distal housing portion 114 and the body wall 174, which helps to secure and mitigate proximal movement of the port housing 106 to and through the body wall 174).

As shown in FIG. 5, the PPIS 101 can additionally include a percutaneous body portion extension 540, which can be added to the proximal end of the percutaneous body portion 112 and secured through a threaded connector 541 to allow for the adjustment of gross changes in body wall thickness of the body wall 174 of the patient over time.

In some implementations, the distal port catheter end 118 can be configured to be sutured to an adjacent tissue 178, 182 or target tissue 176 in the body cavity 172. For instance, the distal port catheter end 118 can include a collar 219 that is configured to resist collapsing of the outer lumen 111 at the distal port catheter end 118. In addition, the distal port catheter end 118 of the PPIS 101 can further include one or more suture haptics 212 that are configured to receive a suture coupled to an adjacent tissue 178, 182 or a target tissue 176.

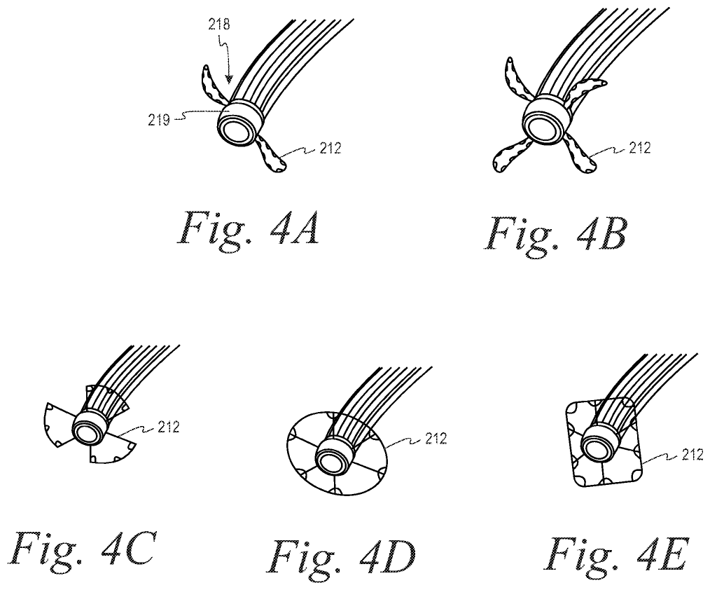

As example in FIGS. 4A-4E, one or more suture haptics 212 can be selected from among a group consisting of: a circle shape, an oval shape, a polygonal shape, and a nonpolygonal shape. The suture haptics 212 can be planar sheets and/or tubular rods or strings which are flexible, semi-rigid or rigid.

In some examples, the port tube 116 can be a self-supporting tube that resists collapsing on itself when the outer lumen 111 is unoccupied. This can be beneficial when the distal port catheter end 118 is not able to be sutured within the body cavity 172 and/or when it is advantageous for the distal port catheter end 118 to float within the body cavity 172.

In other examples, the port catheter 104 of the PPIS 101 can includes an elastic material such that the port tube 116 is configured to collapse on itself when the outer lumen 111 is unoccupied and radially expand when the outer lumen 111 is occupied. In the example shown in FIG. 2A, the port catheter 104 also includes a port catheter stiffener 210 that resists the port tube 116 stretching in the axial direction between the proximal port catheter end 105 and the distal port catheter end 118 while allowing the port tube 116 to collapse on itself when the outer lumen 111 is unoccupied and radially expand when the outer lumen 111 is occupied. Example advantages of this type of configuration are that it allows for significant strain relief of the conduits especially for larger conduits and the ability to provide more circuitous access to tissues such as the pancreas 280 deep within the body 170 and body cavity 172 such as the lesser sac 282 allowing for more body entry point options for the Healthcare Team 140. It also allows for greater body and tissue movement such as bending, twisting, breathing and/or peristalsis that helps resist dislocation of the PPIS 101 or impedance of body tissue movement while providing more comfort to the patient.

In some examples, the port catheter stiffener 210 of the PPIS 101 can be formed from at least one material selected from a group consisting of: metal, glass, quartz or a non-elastic polymer. In other examples the stiffener can be an optical fiber and/or an optical fiber bundle. The optical fiber or optical fiber bundle can be at least one device selected from a group consisting of: an imaging device, a light delivery device, a thermal delivery device, and a biosensor. In another examples, the port catheter stiffener 210 of the PPIS 101 can be one or more electrical wires configured to deliver power and/or provide communication.

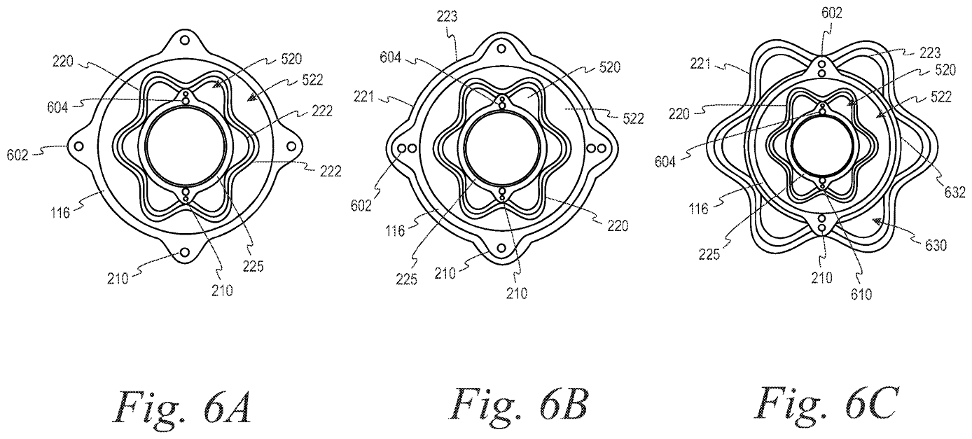

In other examples, as shown in FIGS. 2A, 5, and 6A-6D, the port tube 116 of the PPIS 101 can include one or more port tube wall channels 602 (shown in FIGS. 6A-6D). The port tube wall channels 602 can be configured for the delivery of at least one device selected from the group consisting of an electrical device, an insufflation/deflation device, a mechanical cable device, an active steering device, a fluid delivery device, an optical device, an imaging device, an ablation device, a light delivery device, and a biosensor.

FIG. 2B depicts a perspective view of an implementation of the plug catheter 150, according to an example. As shown in FIG. 2B, the plug catheter 150 can include the plug tube 225 that defines the plug lumen 158 extending between the proximal plug end 152 and the distal plug end 154.

In some examples, one or more portions of the plug tube 225 are collapsible when not in use. The advantages of this type of configuration are that it allows for significant strain relief of the conduits especially for larger conduits and the ability to provide more circuitous access to tissues such as the pancreas 280 deep within the body 170 and body cavity 172 such as the lesser sac 282 allowing for more body entry point options for the Healthcare Team 140. It also allows for greater body and tissue movement such as bending, twisting, breathing and/or peristalsis that helps resist dislocation of the PPIS 101 or impedance of body tissue movement while providing more comfort to the patient.

In other examples, the plug tube 225 of the plug catheter 150 and/or the port tube 116 of the port catheter 104 be a self-supporting tube that resists collapsing on itself when the plug lumen 158 is unoccupied. This can beneficially allow for applications where the distal port catheter end 118 is not able to be sutured within the body cavity 172 or when it is advantageous for the distal port catheter end 118 to float within the body cavity 172.

In some examples, the plug tube 225 can include one or more plug wall channels 604 (shown in FIGS. 6A-6D). The plug wall channels 604 can be configured for the delivery of at least one device selected from the group consisting of an electrical device, an insufflation/deflation device, a mechanical cable device, an active steering device, a fluid delivery device, an optical device, an imaging device, an ablation device, a light delivery device, and a biosensor.

FIG. 2C depicts a perspective view of an implementation of the percutaneous port implant system in the closed configuration, according to an example. As shown in FIG. 2C, the plug catheter 150 is inserted in the PPIS lumen 113 of the PPIS 101. As such, the plug catheter 150 extends through the port housing 106 and the port catheter 104.

In this example, the port catheter 104 includes the closed window 202 at the distal port catheter end 118. The distal plug end 154 of the plug catheter 150 extends to the distal port catheter end 118. Additionally, as shown in FIG. 2C, the port housing 106 can include a locking mechanism that is configured to axially retain the plug catheter 150 in the PPIS lumen 113. For instance, in FIG. 2C, the PPIS 101 includes a slide lock 230. The slide lock 230 can be retracted radially outward during insertion of the proximal plug end 152 distally into the proximal housing portion 110. Once the proximal plug end 152 is fully seated in the proximal housing portion 110, the slide lock 230 can be moved radially inward over a proximal surface of the proximal plug end 152 to axially retain the plug catheter 150 in a fully seated position in the PPIS lumen 113.

FIG. 2D depicts a perspective view of an implementation of the percutaneous port implant system in the closed and capped configuration, according to an example. As shown in FIG. 2, the protective cap 156 covers the proximal opening 107 of the port housing 106 and the proximal plug end 152 of the plug catheter 150. As shown in FIGS. 2D and 5, in some examples, the cap 156 can includes an external electrical connectors 240, an external liquid crystal display (LCD) 238, internal electrical connectors, and/or an internal power induction coil 518 configured to provide auxiliary power and a data connection to the power source 510 and the processor 130, and is configured to activate an aseptic maintenance system 121 when the cap 156 is fully seated over the port housing 106.

As examples, FIGS. 2E-2G depict an implementation of the PPIS 101 positioned and implanted in a human body 170. In particular, FIG. 2E depicts an implementation of the percutaneous port implant system positioned implanted in a human body and an endoscope accessing a body cavity of the human body through the percutaneous port implant system, according to an example. FIG. 2F depicts an implementation of the percutaneous port implant system positioned implanted in the human body and a plug catheter accessing the body cavity of the human body through the percutaneous port implant system, according to an example. FIG. 2G depicts an implementation of the percutaneous port implant system positioned implanted in the human body with a cap coupled to the percutaneous port implant system, according to an example.

FIG. 2E depicts a cross section of the body 170 and a body cavity 172 which includes: a pancreas 280, a lesser sac 282, a greater sac 284, a abdominal wall 286, a gastrocolic ligament 288, a stomach 290, a transverse colon 292, a transverse mesocolon 294, a duodenum 296, and a liver 298. The PPIS 101 has been implanted within the body 170, through the abdominal wall 286, the gastrocolic ligament 288 into the lesser sac 282 between the stomach 290 and the transverse colon 292. In the example shown in FIG. 2E, a distal port catheter end 118 of the port catheter 104 is includes one or more suture haptics 212 that have been sutured to the transverse mesocolon 294 of the lesser sac 282. In other examples, the suture haptic(s) 212 can be sutured to other anatomical structures. As example FIG. 2E, the PPIS 101 is in the open system configuration (FIG. 1A) and is configured to treat a target tissue 176 of the pancreas 280 with an endoscope 299. In other examples, diagnostics and/or therapeutics can be inserted through the PPIS lumen 113 in combination with other devices integrated into the PPIS 101 and/or external to the PPIS 101 as part of the SDT 100 as will be discussed later. Once treatment is complete, the plug catheter 150, cap 156 and over bandage 159 are reinstalled, sealing and reestablishing aseptic environmental integrity of the PPIS 101 using the AMT 121.

FIG. 2F depicts the PPIS 101 in the closed system configuration of FIG. 1B and is configured to treat a target tissue 176 of the pancreas 280 through a self-healing window 202. In examples, diagnostics and/or therapeutics can be inserted through a plug lumen 158 in combination with other devices integrated into the PPIS 101 and/or external to the PPIS 101 as part of the SDT 100 as will be discussed later. Once treatment is complete, standard disinfection solutions and disinfection protocols can be used to disinfect the proximal side of the self-healing window 202 and the plug lumen 158 which works in combination with the AMT 121 to create and maintain the aseptic environmental integrity of the PPIS 101.

FIG. 2G depicts a PPIS 101 in the closed and capped system configuration of FIG. 1C and is configured to treat a target tissue 176 of the pancreas 280 with the cap 156 using UV-C light and an electrochemical over bandage 159 (not shown) to create and maintain the aseptic environmental integrity of the PPIS 101. In examples, diagnostics and/or therapeutics can be in combination with other devices integrated into the PPIS 101, residing in the plug lumen 158 and/or external to the PPIS 101 as part of the SDT 100 as will be discussed later.

Now referring to FIGS. 2C and 5, the PPIS 101 can additionally include a tissue integration element 236 having a first end coupled to the proximal flange 514 and a second end coupled to the distal flange 515 such that the tissue integration element 236 extends between the proximal flange 514 and the distal flange 515. The tissue integration element 236 can be at a position that is laterally outward of an external surface of the percutaneous body portion 112 such that a gap is defined between the tissue integration element 236 and the external surface of the percutaneous body portion 112. In some examples, the gap can be approximately 1 millimeters to approximately 10 millimeters. This can help to stabilize the port housing through the body wall 174 and reduce infection risk.

In some implementations, the tissue integration element 236 can include a plurality of apertures that are configured to allow a tissue of the wall 174 of the body cavity 172 to extend through the tissue integration element 236 into the gap between the tissue integration element 236 and the external surface of the percutaneous body portion 112. For instance, the tissue integration element 236 can include at least one structure selected from a group consisting of: a mesh, a lattice, a weave, and a porous structure.

As shown in FIG. 5, the PPIS 101 can further include one or more tissue illumination sources 532A-532C configured to illuminate the wall 174 of the body cavity 172. As examples, the tissue illumination sources can be configured to emit light (e.g., UV light) to reduce a risk of infection. In some implementations, the tissue integration element 236 can include an optically transmissive material, and the one or more tissue illumination sources 532A-532C can be in optical communication with the tissue integration element 236. In this arrangement, the tissue integration element 236 can be configured to: receive light from the one or more tissue illumination sources 532A-532C, and output light to (i) a first portion of the wall 174 of the body cavity 172 and an external surface of the percutaneous body portion 112 of the port housing 106. This can help to provide the light to tissue that is adjacent to and/or extends through the tissue integration element 236, and/or tissue immediately adjacent to the percutaneous body portion 112 for photodynamic disinfection percutaneous body portion 112. Many microbial pathogens carry endogenous photosensitizer compounds which upon light illumination, inhibit and destroy the microbial pathogens by production of ROS where the wavelength of the light source 532A-532C matches the absorption wavelength of the endogenous photosensitizer compounds.

In some implementations, the tissue integration element 236 can include a plurality of waveguide diffusers 250 that are configured to direct the light out of the tissue integration element 236 to the wall 174 of the body cavity 172 and the external surface of the percutaneous body portion 112 of the port housing. In some implementations, the one or more tissue illumination sources 532A-532C and the tissue integration element 236 are configured to transmit light through the wall 174 of the body cavity 172 to illuminate an external surface of the percutaneous body portion 112 of the port housing 106. In some implementations, the one or more tissue illumination sources 532A-532C include a first tissue illumination source optically coupled to a proximal end of the tissue integration element 236 and a second tissue illumination source optically coupled to a distal end of the tissue integration element 236. In some implementations, the one or more tissue illumination sources 532A and 532B are coupled to the port housing 106 and configured to emit light to an exterior surface of the wall 174 of the body cavity 172.

FIGS. 2B, 3A-3B, 5-6C, 7A-7C, and 11 depict features of the AMT 121 (shown in FIGS. 1A-1C) for establishing, reestablishing, and maintaining aseptic integrity of the PPIS 101 over weeks, months and years by inhibiting and eliminating biofilms, according to examples. During exchanges of plug catheters 150 with the PPIS 101, the PPIS lumen 113 of the PPIS 101 is exposed to biological fluids and biological contaminates of the body 170. Therefore all the surfaces of the system need to reestablish an aseptic environment when a new plug catheter 150 is replaced. In particular, FIGS. 2B, 3A-3B, 5-6C, 7A-7C, and 11 depict features of the PPIS 101 and/or the AMT 121 that can provide for combinations of three-electrode electrochemical oxidation/reduction configurations, ultraviolet-C (UV-C) light emitting diode (LED) light illumination configurations, and/or photodynamic disinfection configurations using photosensitizer compounds with light illumination on the external and internal parts of the PPIS 101.

Within examples, the PPIS 101 can include a three-electrode electrochemical system, which can include a (i) working electrode 222A, 223A, (ii) a counter electrode 222B, 223B and (iii) a reference electrode 222C, 223C on one or more polymer membranes 220, 221, which couple to the internal and/or the external surfaces of the PPIS 101 as will be discussed in detail below. When activated, the three-electrode electrochemical system creates an oxidation/reduction reaction on the surface of the electrodes 222A-222C, 223A-223C with salt-based fluids and/or salt-based hydrogels in contact with the internal and/or external surfaces of the PPIS 101. The salt-based fluids and/or salt-based hydrogels can be (i) naturally occurring in the body 170 (e.g., bodily fluids in the body cavity 172), (ii) supplied to the PPIS 101 from one or more external fluid sources, and/or (iii) integrated in one or more components of the PPIS 101. The oxidation/reduction reaction at the internal and/or external surfaces of the PPIS 101 can produce various reactive oxygen species (ROS) and oxidizing compounds such as hydrogen peroxide and hypochlorous acid, which inhibit and destroy biofilm formation. The PPIS 101 can further include one or more light sources that can emit UV-C light to enhance disinfection of the internal and/or external surfaces of the PPIS 101 and work to damage and/or destroy nucleic acids and disrupt the DNA of the microbial pathogens. For instance, in some implementations described in further detail below, the PPIS 101 can provide for photodynamic disinfection using photosensitizer compounds with light illumination inhibit and destroy biofilm formation by production of ROS as well.

As described above, the PPIS 101 can include the electrodes 222A-222C, 223A-223C one or more polymer membranes 220, 221. In some examples, the electrodes 222A-222C can all be included on a single polymer membrane 220, 221. In other examples, the electrodes 22A-222C can be included on two polymer membranes 220, 221 that are coupled back-to-back to each other.

FIGS. 3A-3B depict additional implementations of the PPIS 101, according to further examples. In these examples, PPIS 101 further includes a membrane 221 on a distal surface of the inflatable bumper balloon 234, and the membrane 221 includes a plurality of electrodes 223. For instance, the plurality of electrodes 223 can include a working electrode 223A, a counter electrode 223B, and a reference electrode 223C. As examples, the working electrode 223A, the counter electrode 223B, and the reference electrode 223C can be formed from at least one electrically conductive material selected from a group consisting of: platinum, silver/silver chloride, carbon, graphene, zinc, bismuth, and copper.

In some examples, the electrodes 223 can be electrically coupled to a power source 510 (shown in FIG. 5) in the port housing 106. For instance, the port housing 106 can include one or more electrical contacts 217, 516 that can be electrically coupled to one or more electrical contacts of the membrane 221. In other examples, the power source 510 can be external to the port housing 106 and/or the power source can be included in the plug catheter 150.

In FIG. 3A, the PPIS 101 includes the inflatable bumper balloon 234 and the membrane 221 with the electrodes 223 at a distal surface of the proximal housing portion 110. An aqueous gel containing salts of NaCl, KCl and or CaCl is applied between the inflatable bumper balloon and the body wall. When the AMT system 121 is activated, an oxidation/reduction reaction is created within the gel and the surfaces of the electrodes 223 between the bumper balloon 234 and the external surface of the body wall 174 which helps to prevent tunnel infections.

As shown in FIG. 3B, the PPIS 101 can additionally or alternatively include an inflatable bumper balloon 235 and/or the membrane 221 with the electrodes 223 at the distal housing portion 114. Additionally, as shown in FIG. 3B, the port tube 116 can include the electrodes 223 as will be described in further detail below.

As shown in FIGS. 2B, 5, and 6A-6C, the PPIS 101 can additionally or alternatively include one or more membranes 220, 221 along the plug tube 225 and/or the port tube 116 to provide for disinfection by the electrodes 222A-222C along the internal and/or external surfaces of the plug catheter 150 and/or the port catheter 104. In each of the implementations shown in FIGS. 2B, 5, and 6A-6C, the membrane(s) 220, 221 can include the plurality of electrodes 222A-222C, and the plurality of electrodes 222 can include the working electrode 222A, the counter electrode 222B, and the reference electrode 222C. As described in further detail below, in some examples, the membrane(s) 220, 221 can additionally assist in defining one or more channels for receiving the fluid and/or salt along the internal and/or external surfaces of the plug catheter 150 and/or the port catheter 104.

As shown in FIG. 5, the PPIS 101 can include a proximal sealing element 502, 503 that is configured to form a fluid-tight seal (i) between an interior surface of the port housing 106 at the proximal housing portion 110 and an outer wall of the plug catheter 150 and (ii) around a circumference of the plug catheter 150 when the plug catheter 150 is inserted in the PPIS lumen 113. In an example, the proximal sealing element of the PPIS 101 can include a first O-ring 502 at the proximal housing portion 110. In some examples, the proximal sealing element can further include a second O-ring 503 that is proximal of the first O-ring 502. The first O-ring 502 and second O-ring 503 ensure a tight and stable seal with the second O-ring 503 as a backup seal to the first O-ring 502.

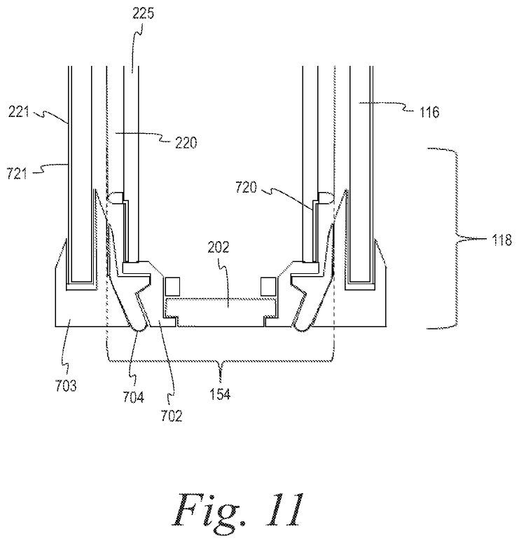

As shown in FIGS. 7A-7D and FIG. 11, the PPIS 101 can also include a distal sealing element 704 configured to form a fluid-tight seal (i) between the distal port catheter end 118 and the plug catheter 150 and (ii) around a circumference of the plug catheter 150 when the plug catheter 150 is inserted in PPIS lumen 113. In one implementation, the distal sealing element 704 of the PPIS 101 can include a wiper seal between the distal plug end 154 and the distal port catheter end 118.

Referring to FIG. 11, the distal sealing element 704 and the axial retention mechanism 700 of the PPIS 101 of the PPIS 101 are configured to be an electrochemical sealing system which produce an oxidation reduction reaction within the seal which kill and inhibit the formation of biofilms from the biological contaminates that might get trapped in the distal sealing element 704 and the axial retention mechanism 700 of the PPIS 101. This includes the distal sealing element 704, the plug catheter distal tip 702, port catheter distal tip 703 and the parts of the axial retention mechanism 700. The distal sealing element 704 can be configured with a hydrogel containing salts which produce hypochlorous acid and other reactive oxygen species using different configurations of electrodes for of AMT system 121 at the interfaces between the distal sealing elements and the axial retention mechanism 700 of the PPIS 101. The distal plug end 154 includes a first electrode 720 that electrically couples to the plug catheter distal tip 702 to the membrane electrodes 220. The first electrode 720 can be configured as a working electrode 222A, a counter electrode 222B, or a reference electrode 222C. The plug catheter distal tip 702 is also electrically coupled to the power source 510 through the membrane as previously described. The distal port catheter end 118 includes a second electrode 721 that electrically couples to the port catheter distal tip 703. The second electrode 721 can be configured as a working electrode 223A, a counter electrode 223B, or a reference electrode 223C. The port catheter distal tip 703 is also electrically coupled to the power source 510 through the membrane as previously described. Similarly, the various electrically conducting parts of the axial retention mechanism 700 are coupled through the first electrode 720 and/or the second electrode 721 to the power source 510. The first electrode 720, second electrode 721, the plug catheter distal tip 702, the port catheter distal tip 703, the spring 706 and other parts of the axial retention mechanism can be formed or coated from at least one electrically conductive material including platinum, titanium, silver/silver chloride, carbon, graphene, zinc, bismuth, and copper. The distal sealing element 704 is formed from a hydrogel which contains one or more salts including NaCl, KCl, and CaCl.

When the AMT system 121 is activated, an oxidation/reduction reaction is created between the plug catheter distal tip 702 and the port catheter distal tip 703 across and through the distal sealing element 704 and along the axial retention mechanism 700 inhibiting and eliminating biofilms on their surfaces. An advantage of this system is that even if the distal sealing elements leak, the electrochemical system still functions to produce hypochlorous acid and other reactive oxygen species which kills any infiltrating pathogens and inhibits biofilm formation along the seal.

As shown in FIG. 5, the proximal plug end 152 can include a flushing port 507 that is configured to receive a fluid into a channel (i) between an outer surface of the plug catheter 150 and an inner surface of the port catheter 104 and (ii) between the proximal sealing element 502, 503 and the distal sealing element 704. The proximal sealing element 502, 503 and the distal sealing element 704 can thus function to reduce migration of pathogens into the PPIS 101 and provide a fluid-tight seal to retain the fluid within the PPIS 101.

As shown in FIGS. 5 and 6A-6C, the plug catheter of the PPIS 101 can include a membrane 220 coupled to an outer surface of the plug tube 225 such that the membrane 220 is between the outer surface of the plug tube 225 and an inner surface of the port tube 116 when the plug catheter 150 is inserted in the port catheter 104. The membrane 220 can extend at least from the distal port catheter end 118 to a position in the port housing 106. Additionally, in FIGS. 6A-6C, the inner surface of the membrane 220 and the outer surface of the plug tube 225 define one or more first channels 520 extending along a portion of the plug catheter 150 between the proximal plug end 152 and the distal plug end 154. For instance, the membrane 220 can be fluted to allow for a more even distribution of flushing the axial retention mechanism 700 (discussed later) around the radius of the plug tube 225.

As shown in FIG. 5, the flushing port 507 can include a fluid port inlet 504 and a fluid port outlet 505. The fluid port inlet 504 can be fluidly coupled with the one or more first channels 520 of the plug catheter 150. The one or more first channels 520 are fluidly coupled with a second channel 522 between an inner surface of the port tube 116 and an outer surface of the membrane 220. The fluid port outlet 505 can be fluidly coupled to the second channel 522. In this arrangement, the fluid that is received at the fluid port inlet 504 is configured to flow along the one or more first channels 520 to the second channel 522, from the second channel 522 to the fluid port outlet 505, and egress out through the fluid port outlet 505. Additionally, in this arrangement, the plurality of electrodes 222A-222C of the PPIS 101 are configured to apply electrical energy to the fluid in the one or more first channels 520 and the second channel 522 to disinfect the plug catheter 150 and the port catheter 104 (e.g., via an oxidation/reduction reaction, which produces various reactive oxygen species (ROS) and oxidizing compounds (e.g., hydrogen peroxide and/or hypochlorous acid).

In another example, the proximal sealing elements 502, 503 can be configured to be an electrochemical seal configured to produce an oxidation reduction reaction within the proximal sealing element 502, 503 between an interior surface of the port lumen 108 of the proximal housing portion 110 an exterior surface of the proximal plug end 152. A plurality of electrodes 222 can be configured to be on an outer wall of the proximal plug end 152 at the proximal sealing element 502, 503 and a plurality electrodes 222 around a circumference of the proximal plug end 152. The plurality of electrodes 222 can be a working electrode 222A, counter electrode 122B and a reference electrode. The proximal sealing elements 502, 503 can be configured with a hydrogel containing salts that produce hypochlorous acid and other reactive oxygen species which kill and or inhibit biofilm formation.

In one implementation, the fluid port inlet 504 can be configured to couple to a fluid source by a fluid-tight seal, and the fluid port outlet 505 is configured to couple to a suction source by a fluid-tight seal. The fluid source and/or the suction source can assist in moving the fluid along the fluid pathway through the first channel(s) 520 and the second channel 522. Within examples, the fluid source can be operated to supply the fluid and/or the vacuum source can be operated to withdraw the fluid before, during, and/or after applying electrical energy to the electrodes 222A-222C. The fluid supplied by the fluid source can be a biocompatible electrolytic solution which can contain various salts selected from a group consisting of: NaCl, KCl, and CaCl with or without biocompatible surfactants, and/or sterile gases, and/or standard disinfection solutions. Examples of biocompatible electrolytic solutions include buffered saline solution, BSS, or Balanced Multielectrolyte Solution, BMES or 0.9% saline solution with or without additional surfactants.

In FIG. 6A, the PPIS 101 includes the membrane 220 coupled to the outer surface of the plug tube 225. In FIGS. 6B-6C, the PPIS 101 also includes the membrane 221 coupled to the outer surface of the port tube 116. In FIG. 6B, the membrane 221 is coupled to the outer surface of the port tube 116. In this example, the membrane 221 and the electrodes 223A-223C can be configured to contact bodily fluids and salt that naturally occur in the body cavity 172. Accordingly, the plurality of electrodes 223A-223C of the membrane 221 can be configured to apply electrical energy to the fluid in the body cavity 172 in contact with the membrane 221 to disinfect the port catheter 104 (e.g., via an oxidation/reduction reaction, which produces various reactive oxygen species (ROS) and oxidizing compounds (e.g., hydrogen peroxide and/or hypochlorous acid)).

In FIG. 6C, the membrane 221 is coupled to the outer surface of the port tube 116 such that the inner surface of the membrane 221 and the outer surface of the port tube 116 define one or more third channels 622 extending along at least a portion of the port catheter 104. In this example, the third channel(s) 622 can additionally or alternatively be configured to receive the fluid (e.g., from the fluid source via the fluid port inlet 504 and/or another fluid source such as the fluid sources described in further detail below with respect to a thermal circulatory bladder system). In FIG. 6C, the plurality of electrodes 223A-223C of the membrane 221 can be configured to apply electrical energy to the fluid in the third channel(s) 622 in contact with the membrane 221 to disinfect the port catheter 104 (e.g., via an oxidation/reduction reaction, which produces various reactive oxygen species (ROS) and oxidizing compounds (e.g., hydrogen peroxide and/or hypochlorous acid). As described in further detail below, the third channel(s) 622 can additionally or alternatively be used as fluid conduits for supplying fluids to other components of the SDT 100 that may be distal of the distal port catheter end 118 (e.g., one or more bladders).

As described above with reference to FIGS. 6A-6C, the membrane 220 can coupled to the outer surface of the plug tube 225 to form the first channel(s) 520 and the second channel 522. However, in other examples, the membrane 220 can additionally or alternatively be coupled to the inner surface of the port tube 116 to form the first channel(s) 520 and the second channel 522.

As described above, the electrodes 222A-222C, 223A-223C are configured to receive electrical power. As such, the PPIS 101 can include one or more features for electrically coupling the electrodes 222A-222C, 223A-223C to a power source. As shown in FIG. 5, for example, PPIS 101 can include a power source 510 in the port housing 106. Providing the power source 510 in the port housing 106 can help to reduce (or, in some implementations, eliminate) external cables and/or wires. In other examples, the power source 510 can be supplemented through the auxiliary power and data connector 240 external to the port housing 106 as auxiliary power for CCM, TMM and CTD and backup power for the PPIS 101.

In some implementations, the membrane(s) 220, 221 can include an electrical connector that can be removably coupled to a corresponding electrical connector on an exterior surface or an internal surface of the port housing 106. This may be beneficial in implementations in which the membrane(s) 220, 221 are removable from the port housing 106 (e.g., in implementations in which the plug catheter 150 includes the membrane(s) 220, 221). For instance, as shown in FIGS. 2A-2B, the plug catheter 150 can include an electrode ribbon connector 224 that can plug into a slot 216 on an external surface of the port housing 106 to electrically couple the electrodes 222A-222C, 223A-223C to the power source 510. In other implementations, the electrode(s) 222A-222C, 223A-223C of the membrane(s) 220, 221 can be electrically coupled to the power source 510 in a non-removable manner. This may be suitable in some implementations in which port catheter 104 includes the membrane(s) 220, 221.

As noted above, the PPIS 101 and/or the AMT 121 can additionally include a light source that can be configured to assist in disinfecting the internal and/or the external surfaces of the PPIS 101. For example, as shown in FIG. 5, the port housing 106 can include a light source 513 that is configured to emit ultraviolet light in the port lumen 108. In one implementation, the port housing 106 can also include an optical element 512 that is configured to diffuse the ultraviolet light in the port lumen 108 of the port housing 106 and an interior surface of the cap 156. Additionally or alternatively, the fluid port cap 226 (e.g., a fluid port cap 226 of the plug catheter 150 shown in FIG. 2B) can include a light source 519 that is configured to emit ultraviolet light to disinfect the fluid port inlet 504 and the fluid port outlet 505. This can help to disinfect an interface between the PPIS 101 and one or more external devices such as, for instance, the fluid source and/or the suction source described above. As shown in FIGS. 2A-2B and 5, port housing 106 can additionally include a connector 218 that is configured to electrically couple to an electrical connector of the light source(s) of the PPIS 101 (e.g., a light ribbon connector 228 of the plug catheter 150 shown in FIG. 2B).

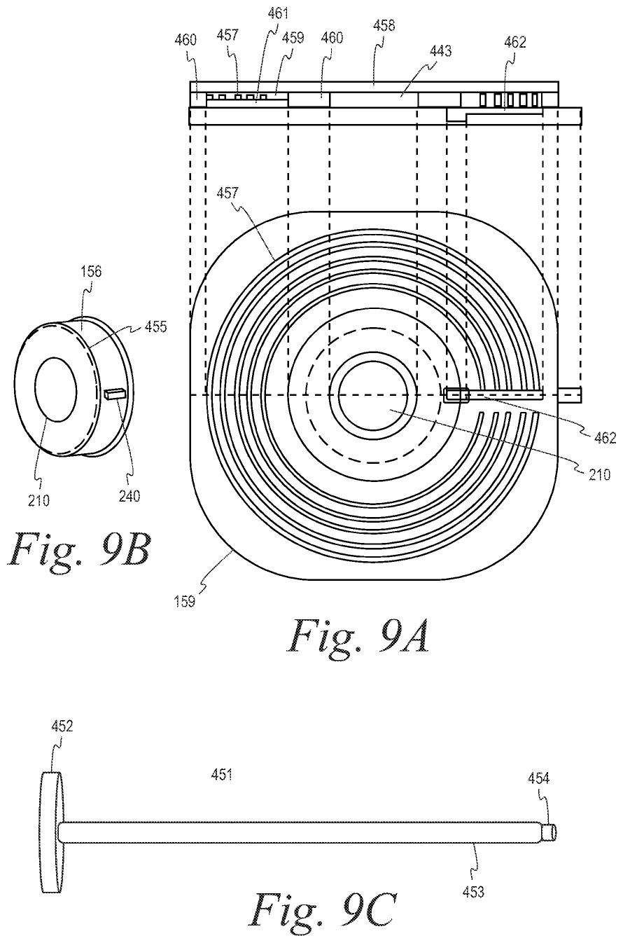

Referring now to FIGS. 1B, 2B, 5, and 9A-9C, the PPIS 101 can include a fill rod 451 for use with configurations (e.g., when then the PPIS 101 is in the closed configuration) using port catheters 104 and/or plug catheters 150 that are collapsible, according to additional examples. As shown in FIG. 9C, the fill rod 451 can include a rod 453 with a tapered distal tip 454 and a flange 452 on a proximal end of the rod 453. The fill rod 451 can be configured to be inserted distally into the plug lumen 158 from the proximal plug end 152 to the distal plug end 154.

After a new plug catheter 150 has been inserted into the PPIS lumen 113 with the plug insertion tool 800 as described below. The PPIS 101 can be flushed and filled with fluid through the fluid port inlet 504 and the fluid port outlet 505. Next, the fluid port inlet 504 can be sealed with a fluid port cap 226. The fill rod 451 can be inserted distally into the plug lumen 158 from the proximal plug end 152 to the distal plug end 154. Any excess fluid exits through the fluid port outlet 505 and the fluid port outlet 505 can be sealed with a fluid port cap 226.

Within examples, the fill rod 451 is configured to consistently maintain a predetermined diameter through the plug lumen 158 of the plug catheter 150 for the insertion and delivery of CCM, TMM and CTD devices through the plug lumen 158 (e.g., in the closed configuration of FIG. 1B and/or the closed and capped configuration of FIG. 1C). Within examples, the fill rod 451 can be of various diameters and lengths.

Within examples, the fill rod 451 can be inserted in the plug lumen 158 (i) after insertion of the plug catheter 150 and flushing fluid as described above, (ii) during operation of the AMT 121, and/or (ii) before the flushing and removal of the plug catheter 150. This can help to maintain the fluid pathways open (e.g., mitigating kinks), and/or mitigate creases to improve coverage of the fluid over the internal and/or external surfaces of the port catheter 104 and/or the plug catheter 150 while applying electrical energy to fluid (e.g., via the electrodes 222A-222C).

Additionally, as shown in FIG. 1C now to FIGS. 5 and 9A-9B, the PPIS 101 can include an over bandage 159 for use with the PPIS 101 in the closed and capped configuration, according to additional examples. The over bandage 159 is configured to be applied over the proximal housing portion 110 and the cap 156 aligning at the cap edge 455, connecting the electrode connector 456 to the auxiliary power & data connector 240 and extending onto the tissue of the body wall 174 external of the body 170 as part of the AMT 121. The electrode connector 456 electrically couple a plurality of electrodes 457 to the power source 510 within the proximal housing portion 110.

As shown in FIGS. 9A-9B, the over bandage 159 can include a flexible polymer substrate first layer 458, a second layer of a membrane 459 with a plurality of electrodes 457 (e.g., the working electrodes 222A, the counter electrodes 222B, and the reference electrodes 222C described above), an adhesive layer 460 radially internal and external to the plurality of electrodes 457, a third layer consisting of a hydrogel layer 461 and a fourth layer including of a connector 462 and a peelable adhesive backing layer 463.

The working electrode 222A, the counter electrode 222B, and the reference electrode 222C of the electrodes 457 can be formed from at least one electrically conductive material selected from a group consisting of: platinum, silver/silver chloride, carbon, graphene, zinc, bismuth, and copper. The plurality of electrodes 457 can have different pattern arrangements with various widths and spacings (example: radial, linear grid, interweaved, etc). The flexible polymer substrate first layer 458 can be formed from a polymer that allows the diffusion of oxygen where the presence of replenishable oxygen enhances the electrolytic and photodynamic antimicrobial effect.

The over bandage 159 can also include a hydrogel layer 461, which can include one or more salts selected from a group consisting of: NaCl, KCl, and CaCl. The hydrogel layer 461 can additionally or alternatively include one or more photosensitizer dyes selected from a group consisting of: methylene blue, toluidine blue, and rose bengal. The hydrogel layer 461 can additionally or alternatively include salts and/or antibiotics and/or impregnated with silver particles and/or a photosensitizer dye or other anti-biofilm inhibiting agents.

In some examples, the light source 532A is configured to illuminate the hydrogel layer 461, and a wavelength of the light source 532A to provide photodynamic disinfection as described above.

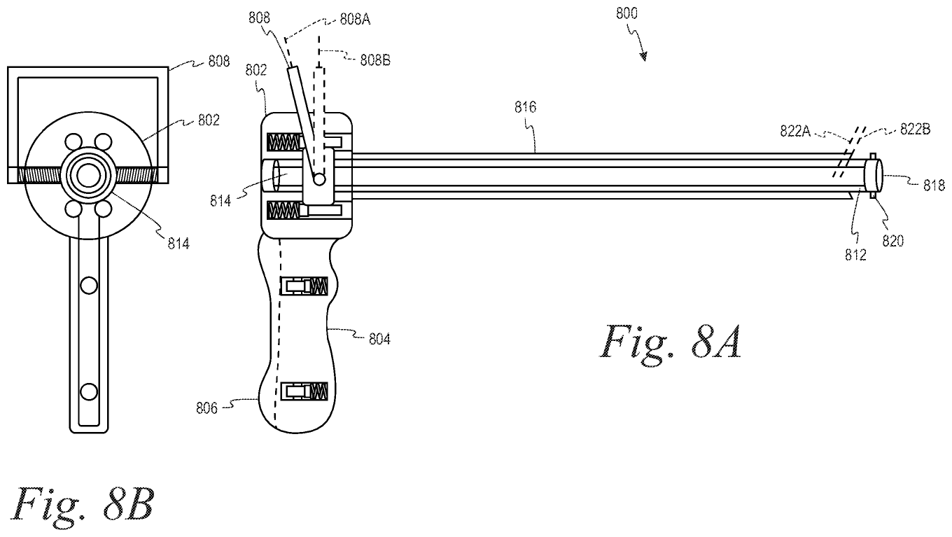

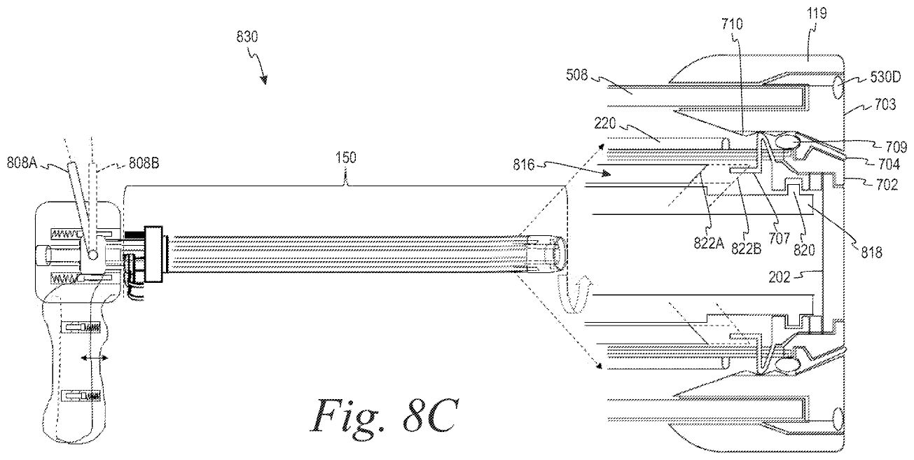

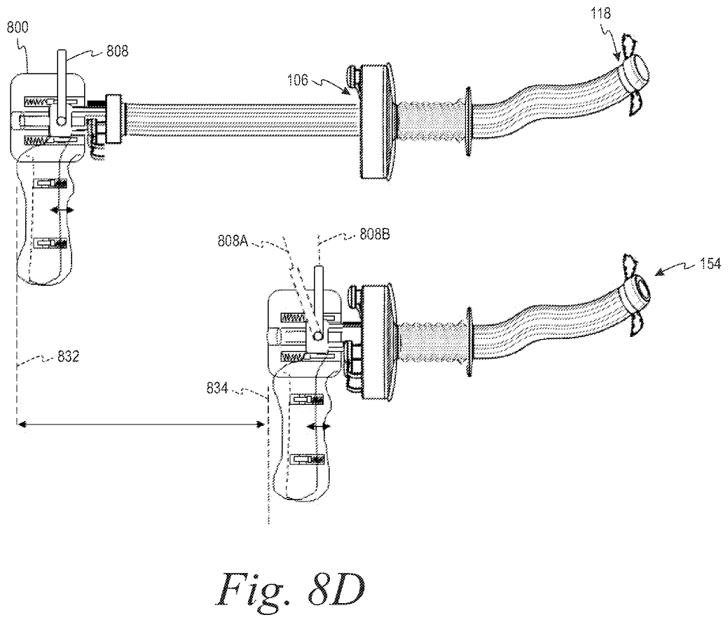

As noted above, in some implementations, the plug catheter 150 can be configured to be inserted in the PPIS lumen 113 of the PPIS 101 such that a distal plug end 154 extends to the distal port catheter end 118. Within examples, the SDT 100 and/or the PPIS 101 can include one or more plug insertion tools and/or axial retention mechanisms to facilitate inserting the plug catheter 150 in the PPIS lumen 113, retaining the distal plug end 154 at the distal port catheter end 118, and/or withdrawing the plug catheter 150 from the PPIS lumen 113. As examples, FIGS. 7A-7D depict an axial retention mechanism 700 and FIGS. 8A-8D depict a plug insertion tool 800 that can be included and/or used with the SDT 100 and/or the PPIS 101.

In the example shown in FIGS. 8A-8C, the plug insertion tool 800 is configured to be inserted in the plug lumen 158 of the plug catheter 150 to form a plug catheter insertion assembly 830. With the plug insertion tool 800 inserted in the plug lumen 158, the plug catheter insertion assembly 830 can move the plug catheter 150 through the PPIS lumen 113 to position the distal plug end 154 at the distal port catheter end 118. For example, as shown in FIGS. 8A-8C, the plug insertion tool 800 can include a handle portion 804 and a shaft portion 812 that extends distally of the handle portion 804.

In general, the handle portion 804 can be configured to facilitate a user gripping and manipulating the plug insertion tool 800 while performing inserting the plug catheter 150 in the PPIS lumen 113 and/or removing the plug catheter 150 from the PPIS lumen 113. For example, the handle portion 804 can have a shape and/or a size that can facilitate a user inserting and/or withdrawing the plug catheter 150 using a single hand. In one implementation, the handle portion 804 can have a shape and/or a size that facilitates the user holding the plug insertion tool 800 in a pistol utensil gripping manner. In another implementation, the insertion tool 800 has a has a tool locking feature 808 configured to lock the position of the user control device relative to the handle portion 804 and the insertion tool housing 802 when moved from an unlocked position 808A to a locked position 808B. This ensures the plug catheter insertion assembly 830 remains in a locked state 822A or 822B as will be discussed below allowing the user easy of operation for one handed use.

The shaft portion 812 can be configured to be inserted in the plug lumen 158 of the plug catheter 150. Additionally, within examples, the plug insertion tool 800 can be substantially stiff in an axial dimension and substantially flexible in a radial dimension such that the plug insertion tool 800 is navigable along a non-linear path of the outer lumen 111 of the port catheter 104 when a portion the plug insertion tool 800 that is external to the port housing 106 is advanced towards the port housing 106.

In some examples, the shaft portion 812 can have a length that is equal to or greater than a length of the plug catheter 150. This can help to position a distal end of the shaft portion 812 at the distal plug end 154.

As shown in FIGS. 7A-7C, the PPIS 101 can further include an axial retention mechanism 700 that is configured to axially retain the distal plug end 154 at the distal port catheter end 118 providing a tight and reliable seal. As described in further detail below, this can assist in operating an axial retention mechanism 700 at the distal port catheter end 118.

The distal port catheter end 118 can include a tapered socket that gradually widens in a direction from the distal port catheter end 118 towards the port housing 106, and the distal plug end 154 can includes a tapered plug that gradually widens in a direction from the distal plug end 154 toward the proximal plug end 152. The tapered plug of the distal plug end 154 can wedge against the tapered socket of the distal port catheter end 118 when the plug insertion tool 800 positions the distal plug end 154 at the distal port catheter end 118. In some examples, the tapered socket and the tapered plug each have a respective conical shape. In examples in which the distal port catheter end 118 and the distal plug end 154 have corresponding tapered shapes, the tapered shapes can help to create a better seal of the distal sealing element 704.

Within examples, the axial retention mechanism 700 can be actuatable by the plug insertion tool 800 between an unlocked state 822B and a locked state 822A. When the axial retention mechanism 700 is in the locked state 822A, the axial retention mechanism 700 resists axial movement of the distal plug end 154 relative to the distal port catheter end 118. By contrast, when the axial retention mechanism 700 is in the unlocked state 822B, the axial retention mechanism 700 allows axial movement of the distal plug end 154 relative to the distal port catheter end 118. Accordingly, in some examples, the plug insertion tool 800 can provide a single tool that can both (i) move the plug catheter 150 through PPIS lumen 113 and (ii) actuate the axial retention mechanism 700 to lock and unlock the distal plug end 154 of plug catheter 150 relative to the distal port catheter end 118. As such, the plug insertion tool 800 can improve operational efficiency and ease of use.

As shown in the example of FIGS. 8A-8C, the plug insertion tool 800 can include an actuator 816 that is configured to interact with the axial retention mechanism 700 to actuate the axial retention mechanism 700 between the locked state 822A and the unlocked state 822B. Additionally, the plug insertion tool 800 can further include a user control device 806 that is external to the port housing 106 and the port catheter 104 when the plug insertion tool 800 is inserted in the plug lumen 158 of the plug catheter 150. The user control device 806 is operable to cause the actuator 816 to interact with the axial retention mechanism 700.

As examples, the axial retention mechanism 700 can include at least one structure selected from a group including: (i) a bayonet coupling between the distal plug end 154 and the distal port catheter end 118, (ii) a threaded coupling between the distal plug end 154 and the distal port catheter end 118, (iii) a spring 706 configured to apply radial compression between the distal plug end 154 and the distal port catheter end 118, (iv) a detent 710 coupling between the distal plug end 154 and the distal port catheter end 118, (v) an inflatable balloon 709 and detent 710 configured to apply both radial and axial compression between the distal plug end 154 and the distal port catheter end 118, and (vi) an inflatable balloon 709 configured to apply radial compression between the distal plug end 154 and the distal port catheter end 118.

FIGS. 7A-8B depict an example implementation in which the axial retention mechanism 700 includes a spring 706 that is configured to extend in a radial direction between the distal plug end 154 and the distal port catheter end 118 to resist axial movement of the distal plug end 154 relative to the distal port catheter end 118 when the axial retention mechanism 700 is in the locked state 822A. In this example, the plug insertion tool 800 is configured to actuate the axial retention mechanism 700 by (i) moving the spring 706 in a radially inward direction to actuate the axial retention mechanism 700 to the unlocked state 822B and (ii) moving the spring 706 in a radially outward direction to actuate the axial retention mechanism 700 to the locked state 822A. For example, the plug insertion tool 800 can be configured to actuate the axial retention mechanism 700 by (i) moving the spring 706 in a radially inward direction to actuate the axial retention mechanism 700 to the unlocked state 822B and (ii) moving the spring 706 in a radially outward direction to actuate the axial retention mechanism 700 to the locked state 822A.

Also, as an example, the spring 706 can include: a spring base 708 that is fixedly coupled to the distal plug end 154, and a spring arm 707 that extends from the spring base 708 to engage the distal port catheter end 118 when the axial retention mechanism 700 is in the locked state 822A. The plug insertion tool 800 can be configured to move a proximal end of the spring 706 towards the spring base 708 to force the spring arm away from the distal port catheter end 118 when the axial retention mechanism 700 is in the unlocked state 822B. And where the user control device 806 engages the actuator 816 when the user control device 806 distally is moved distally toward the handle portion 804 to lock the spring arm 707 away from the distal port catheter end 118 when the axial retention mechanism 700 is in the unlocked state 822B. As shown in FIGS. 8A-8C, the plug insertion tool 800 can include a protrusion 820 and the spring base 708 can include a recess that is configured to receive the protrusion 820 of the plug insertion tool 800 after the plug insertion tool 800 is fully inserted in the plug catheter 150 and rotated relative to the plug catheter 150.

As shown in FIG. 8B, the axial retention mechanism 700 can additionally or alternatively include an inflatable balloon 709 disposed between the distal plug end 154 and the distal port catheter end 118 in another example implementation. The inflatable balloon 709 exerts additional compression force against the detents 710 to the distal sealing element 704 providing a tight and reliable seal.

In operation, the shaft portion 812 of the plug insertion tool 800 can be first inserted in the plug lumen 158 of the plug catheter 150 to form the plug catheter insertion assembly 830. This can include inserting the shaft portion 812 of the plug insertion tool 800 until the distal end of the plug insertion tool 800 is at the distal plug end 154 of the plug catheter 150. The plug insertion tool 800 can then be operated to actuate the axial retention mechanism 700 from the locked state 822A to the unlocked state 822B using the user control device 806 of the plug insertion tool 800. The tool locking feature 808 is then positioned to a locked position 808B locking the plug catheter insertion assembly 830 as previously described.

Next, using the handle portion 804, the plug catheter insertion assembly 830 can be initially inserted into the PPIS lumen 113 to a pre-deployment position 832 (e.g., at the proximal opening 107 of the port housing 106). The handle portion 800A can then be moved distally towards the port housing 106 to move the plug catheter 150 through the PPIS lumen 113 until the distal plug end 154 is at the distal port catheter end 118 to a deployed position 834.

Next, the plug insertion tool 800 can be operated to actuate the axial retention mechanism 700 from the unlocked state 822B to the locked state 822A to axially retain the distal plug end 154 at the distal port catheter end 118. For instance, operating the plug insertion tool 800 to actuate the axial retention mechanism 700 can include operating the user control device of the plug insertion tool 800. While the axial retention mechanism 700 is in the locked state and axially retains the distal plug end 154 at the distal port catheter end 118, the tool locking feature 808 is then positioned to an unlocked position 808A unlocking the plug catheter insertion assembly 830 such that the plug insertion tool 800 can be withdrawn from the plug lumen 158 of the plug catheter 150.

To withdraw the plug catheter 150 from the PPIS lumen 113, the plug insertion tool 800 can be reinserted in the plug lumen 158 of the plug catheter 150. The plug insertion tool 800 can then be operated to actuate the axial retention mechanism 700 from the locked state 822A to the unlocked state 822B (e.g., using the user control device of the plug insertion tool 800). After the axial retention mechanism 700 is actuated to the unlocked state, the plug insertion tool 800 can be moved proximally relative to the port housing 106 to remove the plug catheter 150 from the PPIS lumen 113.

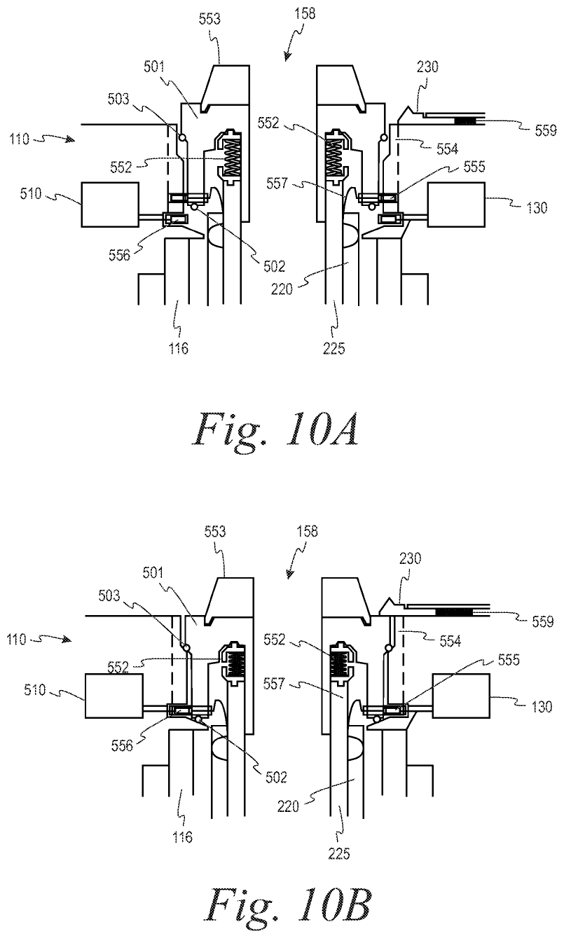

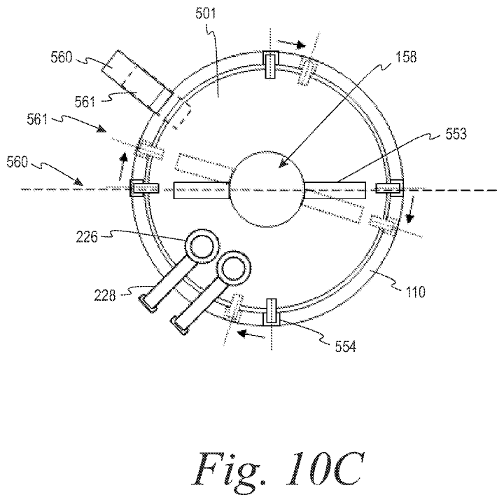

In another example, the PPIS 101 can be configured to use a bayonet connector system 550 providing a sealing mechanism and electrical connections between the plug catheter 150 and the port housing 106. The bayonet connector system 550 is shown in FIGS. 10A-10C as another example of an axial retention mechanism 700.

Referring now to FIG. 10A-10C for PPIS 101 as example for a bayonet connector system for the proximal plug end 152 and the proximal housing portion 110 of the port housing 106. As seen in FIGS. 10A-10C, the percutaneous port implant systems 101B and 101C includes a bayonet connector system 550 configured to form an electrical connection between the port housing 106 at the proximal housing portion 110 and the proximal plug end 152 when the plug catheter 150 is inserted in the port lumen 108. The proximal plug end 152 of the plug catheter 150 contains a constant force spring assembly 552 and is affixed distal to the proximal plug catheter housing 501. Tabs 553 are affixed to the proximal surface of the proximal plug catheter housing 501. As described previously, the percutaneous port implant systems 101B and 101C includes a proximal sealing element configured to form a fluid-tight seal between an interior surface of the port housing 106 around a circumference of the proximal plug end 152 when the plug catheter 150 is inserted in the port lumen 108. The proximal sealing element includes a first O-ring 502, and further includes a second O-ring 503 that is proximal of the first O-ring 502.

The bayonet connector system 550 includes a plug catheter pin connector 555 and a port connector 556. The plug catheter pin connector 555 is connected to the proximal plug catheter housing 551 and the port connector 556 is connected to the proximal housing portion 110. A plurality of membrane electrical contacts includes a ribbon cable 557 configured to provide an electrical connection to couple the plurality of membrane 220 electrical contacts and/or other electrical connects coming on or through the plug tube 225 to the plurality of electrical contacts of the plug catheter pin connector 555.

The plug catheter 150 is inserted distally into the port lumen 108, where the plug catheter pin connectors 555 are aligned with the slots 554 in the proximal plug catheter housing 501 and the slide lock 203 is pushed radially outward against the slide lock spring 559 to a first position 560. The plug catheter pin connector 555 pin moves distally through the slot 554 where the first O-ring 502 and the second O-ring 503 engage the internal walls of the proximal housing portion 110 compressing the constant force spring assembly 552 and aligning plug catheter pin connector 555 with the port connector 556. The proximal plug catheter housing 501 is rotated using the tabs 553 from the first position 560 to a second position 561 providing an electrical connection to the power source 510 and the processor 130 between the plug catheter 150 and the proximal housing portion 110. The constant force spring assembly 552 provides compressional forces at the pin connectors. The slide lock 230 is released and the slide lock spring 559 pushes the slide lock radially inward over the proximal surface of the proximal plug catheter housing 501 locking the plug catheter 150 and the proximal housing portion 110 together. The process is repeated in reverse to remove the plug catheter 150 from the proximal housing portion 110.

In another example, FIG. 11 using the bayonet connector system 550, where the distal port catheter end 118 are either not able to be sutured within the body cavity 172 or when it is advantageous for the distal port catheter end 118 to float within the body cavity 172, the plug tube 225 port tube 116 are semi rigid or minimally compressible both axially and torsionally and both flexible and bendable radially. When the plug catheter 150 is fully inserted through the port catheter 104, the plug catheter distal tip 702 engages the port catheter distal tip 703 compressing the distal sealing element 704. The plug catheter distal tip 702 and the port catheter distal tip 703 can have matching conical aspherical shaped surfaces (not shown) allowing the two surfaces to slide against each other providing both axial and rotation forces and compressing the distal sealing element 704. The rotational force within the constant force spring assembly 552 of the bayonet connector system 550 provides rotational entry, sealing and axial and rotational locking forces.

In some other examples, as shown in FIGS. 14A-14E and 15A-15E, it can be advantageous to have a distal portion of the plug catheter 150 extend distally beyond the distal port catheter end 118 (e.g., which, in some implementations, may be sutured to an anatomical structure). The distal portion of the plug catheter 150 can be formed from materials that are flexible and stretchable which can incorporate tubes, springs and/or wires to allow their independent articulation and active positioning of the distal portion.

As an example, FIGS. 14A-14F depicts a fenestrated tube plug catheter 330 of the PPIS 101 is configured to be inserted in the port catheter 104 such that the distal portion 334 extends distally of the distal port catheter end 118. The fenestrated plug catheter 330 can be used for, for instance, ascites drainage, peritoneal dialysis and/or drug delivery. Delivery or removal of fluids can be accomplished through standard methods of pressure or vacuum created through mechanical means or gravity and valved delivery/removal systems for switching between them as required attached to fluid connectors. Examples can include vacuum bottles, hand pumps, mechanical pumps, spring loaded syringes, hanging bags, etc. The fenestrated plug catheter 330 can be substantially similar to the plug catheter 150 described herein, except for the differences described below.

In some examples, the fenestrated tube plug catheter 330 of the PPIS 101 can further include a fluid tube 338 that extends through the plug lumen 158 of the plug tube 225, where a distal portion 334 of the fluid tube 338 extends distally of the plug tube 225 and where the distal portion 334 of the fluid tube 338 includes a plurality of holes 332. The plurality of holes 332 can be arranged along a longitudinal axis of a distal portion 334 of the fenestrated plug catheter 330 where the distal portion 334 of the plug catheter 150 is configured to extend distally of the distal port catheter end 118. In some implementations, the distal portion 334 can be formed from a shape memory material that biases the port catheter 104 to a predetermined shape when the fenestrated plug catheter 330 extends distally of the distal port catheter end. As examples, the distal portion 334 can be a predetermined shape such as a spiral, a curved shape, an s-shape, a straight shape, a hairpin shape, an irregularly contoured shape or combinations thereof.

In operation, the plug insertion tool 800 can be inserted into the plug lumen 158 of the fenestrated tube plug catheter 330 as previously described above with respect to FIGS. 8A-8C. In this example, the fluid tube 338 extends proximally of the plug insertion tool 800 through a tool lumen 814. Next, an applicator rod 342 is inserted in the fluid tube 335 and used to actuate the fenestrated plug catheter 330 from the predetermined shape to a straight shape 344 to form a fenestrated tube plug catheter insertion assembly 341.

Next, using the handle portion 804 of the fenestrated tube insertion assembly 341, the fenestrated tube insertion assembly 341 can be initially inserted into the PPIS lumen 113. In some implementations, the plug tube 225 can include an indicator 337 that indicates when the insertion assembly 341 has been inserted through the PPIS lumen 113 to a pre-deployment position 346 at which a distal end of the applicator rod 342 is at the distal port catheter end 118. For instance, the indicator 337 can align with the proximal surface of the proximal housing portion 110 when the fenestrated tube plug catheter insertion assembly 341 is at the pre-deployment position 346. While the fenestrated tube plug catheter insertion assembly 341 is at the pre-deployment position 346, the fenestrated tube plug catheter insertion assembly 341 is movable relative to the applicator rod 342 and the port housing 106 to move the fenestrated tube plug catheter insertion assembly 341 distally of the distal port catheter end 118.

A proximal end of the applicator rod 342 can be held in a fixed position, and the fenestrated tube insertion assembly 341 can be inserted over the applicator rod 342 towards the distal end of the port catheter 104 to a deployed position 348 where the distal portion 334 assumes its predetermined shape within the body cavity 172. The fenestrated tube plug catheter insertion assembly 341 can then be locked in place with the plug insertion tool 800 and removed as previously described.

An advantage of this method of deployment for spiraling tubes is that the tip of the tube can be controlled and deployed around itself as the tube retains its spiral shape where the sidewall of the tube expands within the particular body cavity reducing (or minimizing) the potential of perforating the body cavity 172.

In another example, seen in FIG. 14C, the fenestrated tube plug catheter 330 can include a plurality of branches 340 and a plurality of fluid tubes 335A and 335B where one or more applicator rods 342 are configured to be inserted into the fluid tubes 335A and 335B.

In some examples, the plug catheter 150 can have a fixed length. In other examples, the plug catheter 150 can have an adjustable length. For instance, in some examples, a distal portion of the plug catheter 150 can be extensible and/or retractable. This can help to adjust a position of the distal plug end 154 to access a particular target tissue and/or location in the body cavity 172 (e.g., when using a device of the plug catheter 150), and/or retract the distal plug end 154 away from the target tissue and/or the location in the body cavity 172 (e.g., when not using the device of the plug catheter 150). Additionally or alternatively, the plug catheter 150 that is configured to be extensible and/or retractable can provide for accessing a plurality of target tissues and/or locations in the body cavity 172.

FIGS. 15A-15D depict an implementations of the PPIS 101 including the plug catheter 150 that is configured to be extensible and/or retractable, according to an example. FIG. 15A depicts the plug catheter 150 that is configured to be extensible and/or retractable, and FIG. 15B depicts the plug catheter 150 of FIG. 15A inserted in the PPIS lumen 113 of the port housing 106 and the port catheter 104, according to the example.

As shown in FIG. 15A, the plug catheter 150 can include a distal extensible portion 302 that is configured to be extended and/or retracted relative to the distal plug end 154 of the plug catheter 150. As shown in FIG. 15B, the distal extensible portion 302 can extend distally from the distal port catheter end 118 when the port catheter 150 is fully inserted in the PPIS lumen 113.

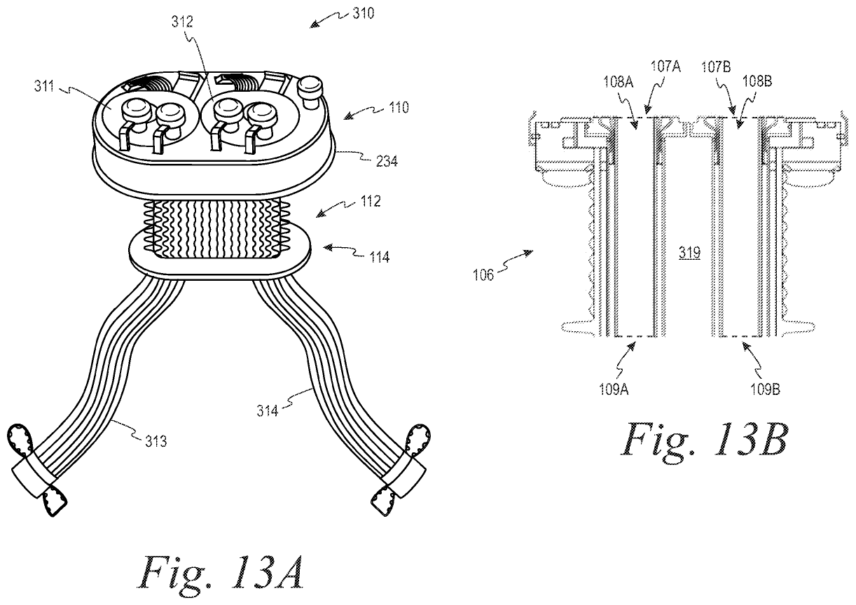

In FIG. 15A, the distal extensible portion 302 of the plug catheter 150 can include a tube formed from an elastic material. The elastic material can be a material having an elasticity that is suitable to allow the distal extensible portion 302 to elastically deform and increase in length when a force is applied to the distal extensible portion 302. In some examples, the elastic material of the distal extensible portion 302 can be further configured such that, when the force is removed, the distal extensible portion 302 returns to its original length. As shown in FIG. 13A, in some examples, the distal extensible portion 302 can further include a biasing element (e.g., a spring 303 and/or elastic bands) that can assist in returning the distal extensible portion 302 to its original length when the force is removed from the distal extensible portion 302. In other examples, the distal extensible portion 302 can omit the biasing element.

In one implementation, the plug catheter 150 can be inserted in the PPIS lumen 113 as shown in FIG. 15B. A device in the plug lumen 158 of the plug catheter 150 can then be moved distally (e.g., relative to the distal plug end 154) to apply a distally directed force on a distal end of the distal extensible portion 302. The distally directed force on the distal end of the distal extensible portion 302 can extend the distal extensible portion 302 from a retracted length to an extended length. When the device in the plug lumen 158 is moved proximally (e.g., relative to the distal plug end 154) to remove the distally directed force from the distal end of the distal extensible portion 302 and responsively cause the distal extensible portion 302 to retract from the extended length back to the retracted length. In implementations that include the biasing element, the biasing element can assist in retracting the distal extensible portion 302 from the extended length to the retracted length.

FIGS. 15C-15D depict the plug catheter 150 of FIG. 15A used with the plug insertion tool 800 described above to insert the plug catheter 150 in the PPIS lumen 113. As shown in FIGS. 15C-15D, this process is substantially similar or identical to the process described above with respect to FIGS. 14AD-14F, except the plug catheter 150 shown in FIG. 15A can omit the fluid tube 335 in some implementations.

Because of the modular nature of the PPIS 101, the PPIS 101 can be configured in many configurations as shown, for example, in FIGS. 12, 13A-13B, 16A-16D, 17A-17B and 18A-18B.

In the example shown and described above (e.g., the examples of FIGS. 1A-1C and 5), the PPIS 101 includes both the port housing 106 and the port catheter 104. In other examples, the PPIS 101 can omit the port catheter 104. In such examples, the port housing 106 can include one or more features described herein for the distal plug end 154 to facilitate using the port housing 106 with the plug catheters 150 described herein. For instance, the port housing 106 can include the closed window 20, the axial retention mechanism 700, and/or the bayonet connector system 550 described herein.

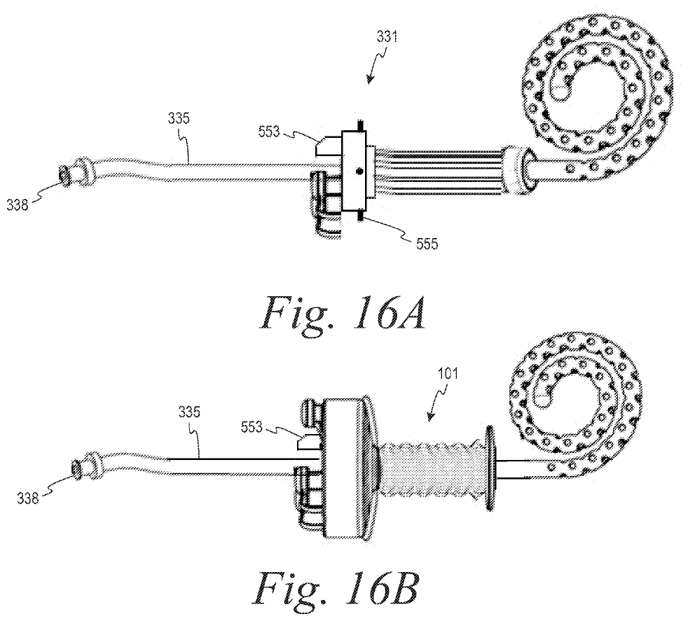

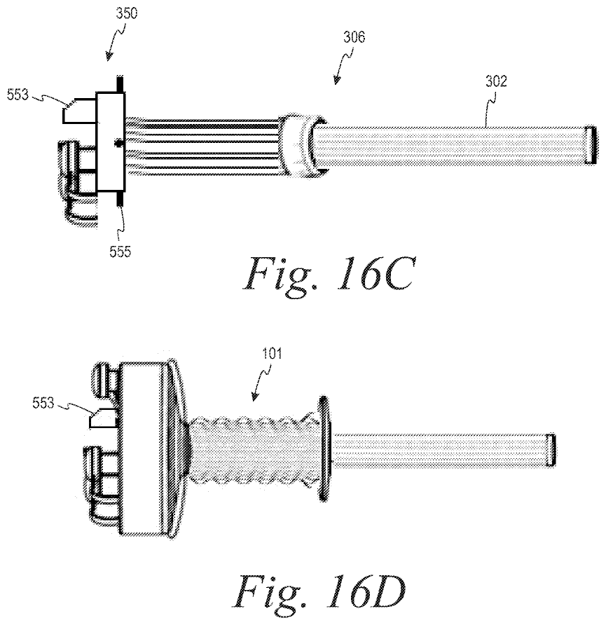

As examples, FIGS. 12 and 16A-16D depict implementations of the PPIS 101 that omit the port catheter 104 and include the port housing 106 with a configuration that can be used where recurring access may only be desired through the body wall 174 not greatly extending into the body cavity 172. In the example shown in FIG. 12, the distal plug end 154 of the plug catheter 150 seals in the distal housing portion 114 of the port housing 106. In the example shown in FIG. 16A and FIG. 16B, a short fenestrated tube plug catheter 331 extends through the port housing 106 and is coupled to the port housing 106 by the bayonet connector system 550 configuration described with respect to FIG. 10. In another example, FIG. 16C and FIG. 16D depicts a short extensible plug catheter 306 extends through the port housing 106 and is coupled to the port housing 106 by the bayonet connector system 550 configuration described with respect to FIG. 10.

In another example, FIG. 13A-13B depict a perspective view of a multiport catheter and multi plug catheter configuration 310 of the PPIS 101.

As shown in FIG. 13A, the multiport catheter and multi plug catheter configuration 310 of the PPIS 101 can include the port housing 106, a first port catheter 313, and a first plug catheter 311 configured to be inserted in the first port catheter 313. The PPIS 101 can also include a second port catheter 314 and a second plug catheter 312 configured to be inserted in the second port catheter 314. The port housing 106 including the proximal housing portion 110, the percutaneous body portion 112, and the distal housing portion 114 of the PPIS 101 can have a cross-sectional shape that is selected from among a group consisting of: a circle shape, an oval shape, a polygonal shape, and a nonpolygonal shape

As shown in FIG. 13B, the multiport catheter and multi plug catheter configuration 310 can further include a second port lumen 108B between the proximal opening 107B and the distal opening 109B, where the second port lumen 108B extends entirely through the proximal housing portion 110, the percutaneous body portion 112, and the distal housing portion 114, where the port lumen 108A and the second port lumen 108B are separated by one or more internal walls 319 of the port housing 106.

In some examples, the multiport catheter and multi plug catheter configuration 310 can treat two locations within one body cavity 172, or one location in one body cavity 172 and another location in an adjacent cavity 172 through a single PPIS 101. For instance, in some examples, the multiport catheter and multi plug catheter configuration 310 can treat two locations in a pelvic cavity, an abdominal cavity, a thoracic cavity, or a cranial cavity through a single PPIS 101. In other examples the multiport catheter and multi plug catheter configuration 310 can treat two locations in an abdominal cavity and pelvic cavity, and an abdominal cavity and a thoracic cavity, through a single PPIS 101. The is particularly helpful for treating metastatic cancer where the cancer has spread to multiple locations within the same cavity or one or more body cavity 172 of the human body 170.

In some examples, for the treatment of metastatic pancreatic cancer, the multiport catheter and multi plug catheter configuration 310 includes the port housing 106 configured to have a collapsible first port catheter 313 that accesses the lesser sac 282 and the pancreas 280 and configured to have a second short PPIS 300 that accesses the greater sac 284. This configuration allows for a wide range of treatment options for metastatic pancreatic cancer patients through a single PPIS 101.

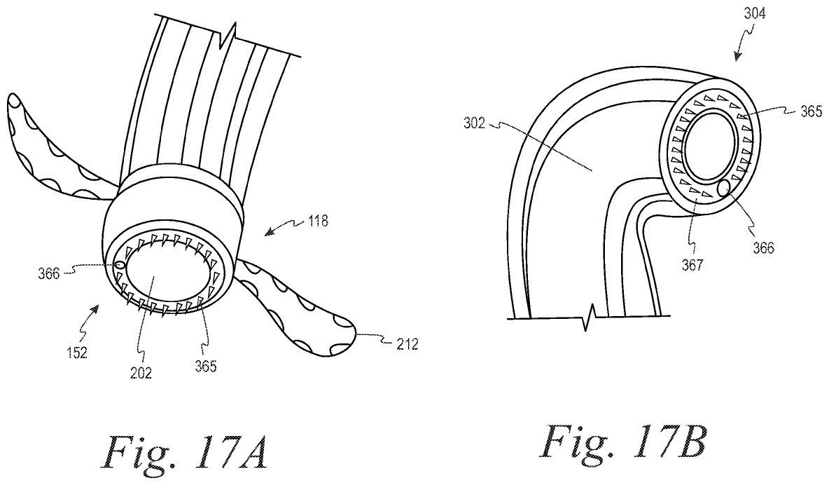

Referring to FIGS. 17A and 17B, the distal plug end 154 and the extensible port catheter distal end 118 can include one or more biosensors 365, one or more imaging devices 751, one or more devices of the CCM, one or more devices of the TMM, and/or one or more devices of the CT, which are configured to be coupled to the face plate 367 of the plug catheter 150 and extensible plug catheter 301. As example, the face plate 367 can be configured with one or more microneedles, one or more piezo electric devices, and/or one or more ultrasonic devices. One advantage of this configuration is a mechanical pressure wave can be directed into the target tissue 176, facilitating fluid movement within the target tissue 176 for therapeutic and diagnostic treatments such as drug delivery using sonodynamic therapy or facilitating drug delivery using single or multiple injection devices and profusion needles as described below.

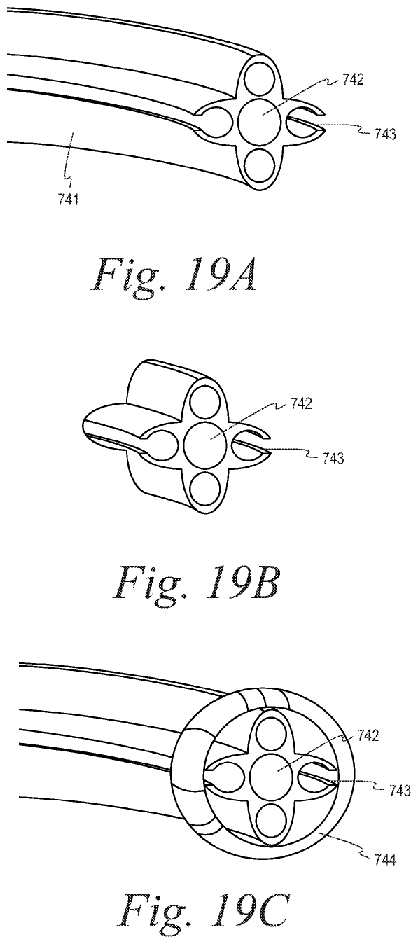

According to an example as seen in FIGS. 19A-19C, the PPIS 101 in the “open” and “closed” configuration seen in FIGS. 1A and 1B includes a lumen applicator 740 configured to be inserted through the plug lumen 158, the extensible plug catheter lumen 301 or the PPIS lumen 113 to treat in, on, adjacent to, and/or distant to a target tissue 176 of the body 170. The lumen applicator 740 is comprised of one or more channels 742 and/or clips 743 for the organization and delivery of one or more devices 750. The lumen applicator 740 is further comprised of a lumen fixation element 744 affixed to the lumen applicator 740 configured to axially retain the distal end of the lumen applicator 740 at the distal port catheter end 118, the distal plug end 154 proximal to the window 202, and the distal extensible portion 302 proximal to the extensible plug catheter window 202. The lumen fixation element 744 is selected from among a group consisting of: balloons, springs, threads, bayonet mechanisms, clips and detents. In other examples, the lumen fixation element 744 can be affixed along the lumen applicator 740 from its proximal end to its distal end. When the lumen fixation element 744 is engaged, the lumen applicator 740 maintains its position within the inner lumen of the plug catheter 150 and/or port catheter 104. When the lumen applicator 740 is disengaged, the lumen applicator 740 can be repositioned or withdrawn from the plug lumen 158 of the plug catheters 150, 301 and the outer lumen of the port catheter 104. In some examples (not shown), two or more lumen applicators 740 with different channels 742 and clips 743 are configured to be used in combination, the first distal inner lumen applicator 740 configured to deliver one or more devices 750 and a second proximal lumen applicator 740 as an axial length stop at the proximal end where the second lumen applicator 740 is larger than the diameter of the plug lumen 158 or port lumen 108 and engages the proximal surfaces of the proximal plug end 152 and/or the proximal housing portion 110.

The lumen applicator 740 is configured to deliver devices which include at least one or more devices 750 selected from a group consisting of: an imaging device, endoscope, an active steering device, an injection device, a biopsy device, a surgical tool, an irrigation device, an insufflation device, a deflation device, an ultrasonic device, an ablation device, a light delivery device, a thermal delivery device, a radiation device, a radiation seed delivery device, a biosensor, a CCM device, a TMM device, and a CTD device. The lumen applicator can be an extrusion or the combination of separate conduits and clips and can have multiple configurations and combinations of channel guides and clips dependent on the combination of devices to be delivered.

According to an example, FIGS. 20A-20I, a lumen applicator 740 is configured to be inserted through the plug lumen 158 of the distal end of the extensible plug catheter portion 302 to the distal portion end 304. The lumen fixation element 744 is engaged to fix the position of the lumen applicator 740 against the proximal surface of the self-healing window 202. One or more therapeutic and/or diagnostic devices are configured to be inserted through one or more channels of the lumen applicator 740. The one or more devices are extended distally through the self-healing window 202 through a puncture site 753 in, on, adjacent or distant to a target tissue 176 to deliver a therapeutic and/or diagnostic treatment. The devices comprise at least one device selected from a group consisting of: an imaging device, endoscope, an active steering device, an injection device, a biopsy device, a surgical tool, an irrigation device, an insufflation device, a deflation device, an ultrasonic device, an ablation device, a light delivery device, a thermal delivery device, a radiation device, a radiation seed delivery device, a biosensor, a CCM device, a TMM device, and a CTD device. Each device's position can be articulated independently or in combination. Once the treatment has been completed, the one or more devices are withdrawn proximally from the target tissue 176 through the puncture site 753 of the self-healing window 202 into the lumen applicator 740. The lumen fixation element 744 is disengaged and the lumen applicator 740 is removed proximally out of the extensible plug catheter 301.

As example in FIG. 20C, a CCM, TMM and CTD combination configuration of the PPIS 101 includes a first device 754 configured as a drug delivery profusion needle device, a second device 755 configured as a drug delivery profusion needle device, a third device 756 configured as a vacuum profusion needle device, a fourth device 757 configured as a vacuum profusion needle device, a fifth device 751 configured as an articulating imaging device and a sixth device configured as a biosensor 365. The first device 754, second device 755, third device 756 and fourth device 757 are extended distally through the self-healing window 202 through a puncture site 753 into the target tissue 176. One or more fluids are delivered through the first device 754 and the second device 755, and a vacuum is placed on the third device 756 and the fourth device 757. An interstitial fluid pressure gradient is produced between the first device 754, the second device 755, the third device 756 and the fourth device 757 of the target tissue to predictably distribute the fluid through the target tissue 176. The fifth device 751 is configured to image the target tissue 176 and assist in position and reposition the distal end of the extensible plug catheter 150 (e.g., shown in FIG. 15A). The sixth device 365 monitors the target tissue 176 before, during, and after the treatment.

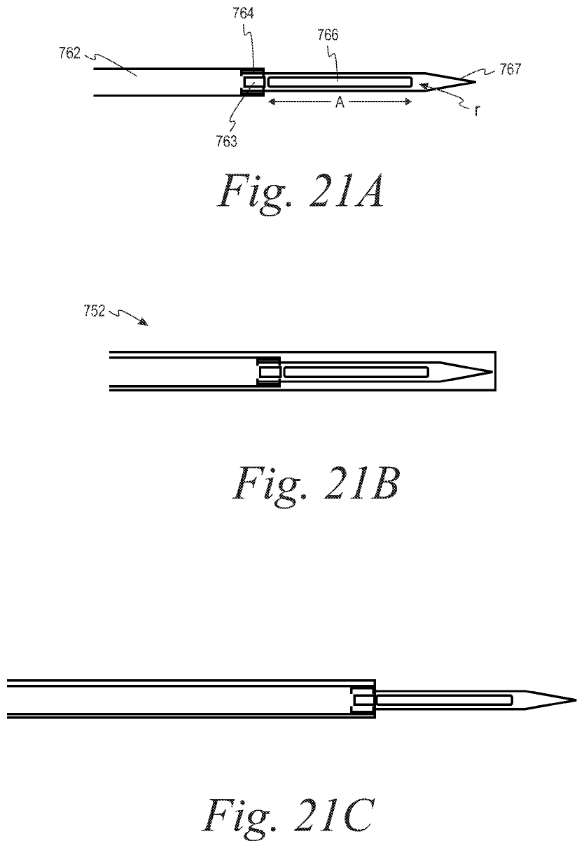

According to an example seen in FIGS. 21A-21C, the PPIS 101 includes the use of a brachytherapy needle probe 760 configured to perform local radiation therapy to a target tissue 176 with the PPIS 101 as previously described. The brachytherapy needle probe 760 includes a cylindrically hollow housing portion 767 with an open threaded proximal end and a sealed conically piercing distal end. The housing can be formed from titanium. Cylindrical radioactive inserts 766 are inserted into the proximal end of the housing portion 767 and sealed with a plug 763. The housing portion 767 is then fixed to the catheter rod 762 by threading it onto the catheter rod. A cylindrical conduit shield 752 is configured with an open proximal end and a closed distal end and can be formed from lead (Pb). The housing portion 767 and catheter rod 762 are inserted into a cylindrical conduit shield 752 to the proximal side of the conduit shield. The brachytherapy needle probe 760 is used with the PPIS 101 and the lumen applicator 740 as previously described.

The cylindrical distal housing portion 767 is defined by a distal inner housing length “A”, an inner housing diameter “r”, with a corresponding a radioactive insert length and radius. The brachytherapy needle probe 760 is configured to provide a specified radiation dose to a targeted tissue 176 of the body 170 and is defined by the housing dimensions “A” and “r”, the measured radiation of the cylindrical radioactive insert 766 at the time of treatment, the time the brachytherapy needle probe 760 and the number and distribution of the brachytherapy needle probe 760 that reside in the target tissue 176 during treatment.

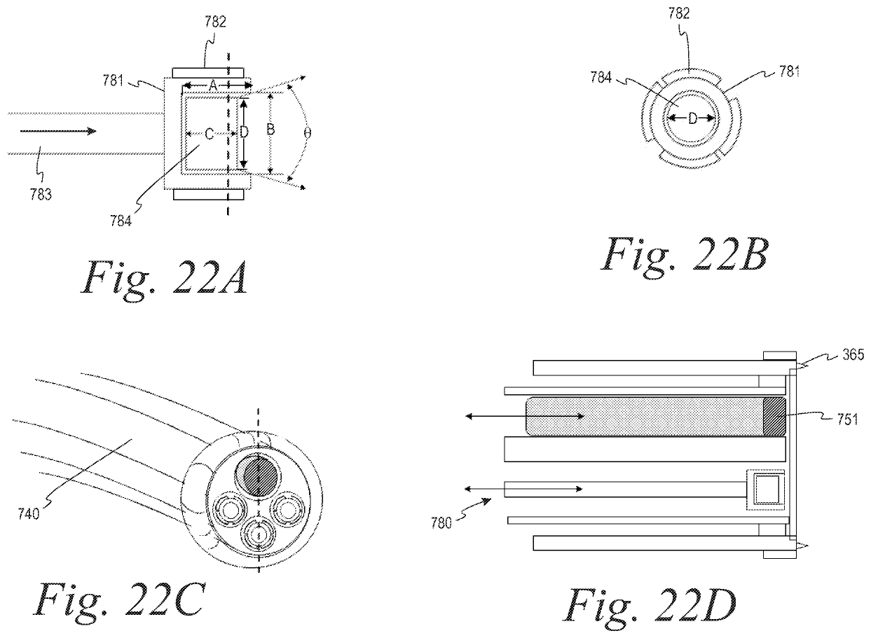

According to an example seen in FIGS. 22A and 22B, the PPIS 101 can include the use of an enface radiating brachytherapy probe 780 configured to perform local radiation therapy to a target tissue 176. Referring to FIG. 22A, the enface radiating brachytherapy probe 780 includes a housing portion 781 defining a cylindrical housing whose opening is at the distal end, one or more spacers 782 rigidly fixed to the external circumferential surface of the cylindrical housing portion 781 and a rod 783 rigidly fixed to the proximal end of the housing portion 781. The housing portion 791 is formed from at least one material selected from a group including: lead (Pb). Tin (Sn), antimony (Sb), tungsten (W) and bismuth (Bi).

In other examples, the housing portion 781, the spacers 782 and the radioactive inserts 784 can have cross sections selected from among a group including: a circle shape, an oval shape, a polygonal shape, and a nonpolygonal shape.

Referring to FIG. 22B, the enface radiating brachytherapy probe 780 is configured to be inserted into the plug lumen 158 of the plug catheter 150 or extended plug catheter 301 and/or one or more channels 742 of the lumen applicator 740 and/or affixed at the distal end of the one or more clips 743 of the lumen applicator 740. An imaging device 751 is inserted through the lumen applicator 740 and is configured to guide and position the enface radiating brachytherapy probe 780 to the target tissue 176. The imaging device 751 can include at least one device selected from a group including: an endoscope, an ultrasonic imaging device, an optical coherence tomography (OCT) imaging device, a brightfield imaging device, a hyperspectral imaging device, and a fluorescence imaging device.

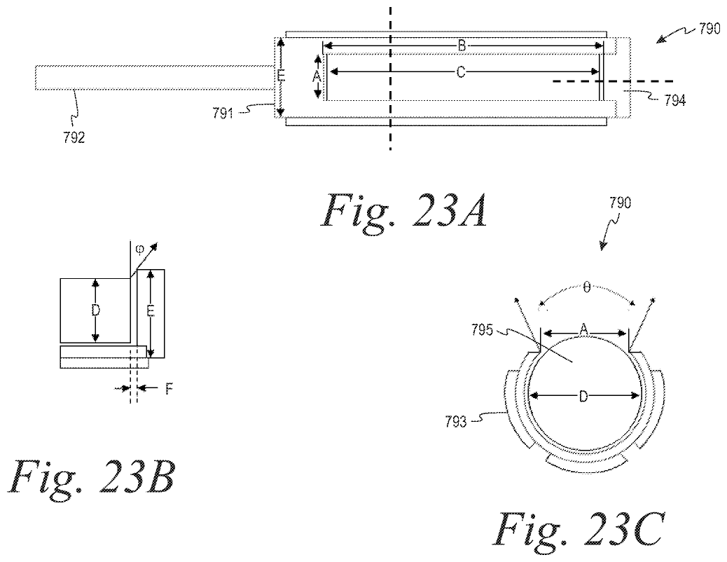

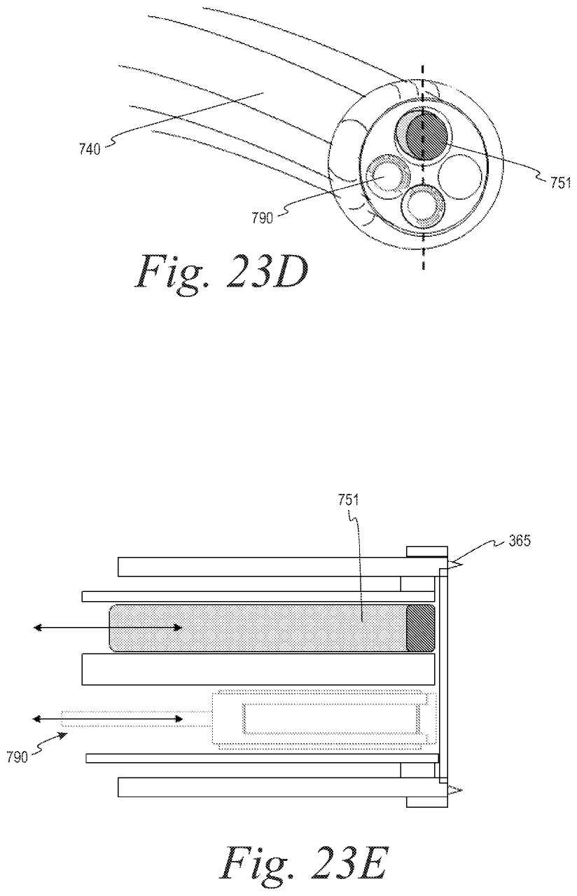

Referring to FIG. 23A, the cylindrical distal housing 781 is defined by a distal inner housing depth—“A”, an inner housing diameter “B”, a radioactive insert thickness “C” and a radioactive insert diameter “D” which defines a specific radiation pattern “θ”. In some examples, cylindrical housing spacers (not shown) are placed distal or proximal to the cylindrical radioactive insert 784, where the spacers can be solid or patterned with one or more holes. The enface radiating brachytherapy lumen probe 720 is configured to provide a specified radiation dose to a targeted tissue 176 of the body 170 and is defined by the housing dimensions “A”, “B”, “C”,“D”, “θ”, the spacers, the measured radiation of the cylindrical radioactive insert 784 at the time of treatment, the distance between the distal surface of the cylindrical radioactive insert 784 and the proximal surface of the target tissue 176, the absorption of radiation by the window 202 of the plug catheter 301 and the time the enface radiating brachytherapy lumen probe 780 and plug catheter 301 resides at the target tissue 176. One or more enface radiating lumen brachytherapy probes 780 can be used to create specific radiation intensity patterns dependent on the size and shape of the target tissue 176. The radiation dose can be extended along a targeted tissue 176 of the body 170 by actively repositioning the extensible plug catheter 301 and brachytherapy probe 780 using the imaging device 751.

According to an example seen in FIGS. 23A-23B, the PPIS 101 can include the use of a side radiating brachytherapy probe 790 configured to perform local radiation therapy to a target tissue 176. Referring to FIG. 23A, the side radiating brachytherapy probe 790 includes a housing portion 791 defining a cylindrical housing opening of the circumferential wall of the housing, one or more spacers 793 rigidly fixed to the external circumferential surface of the cylindrical housing 791 and a rod 792 rigidly fixed to the proximal end of the housing portion 791. The housing portion 791 is formed from at least one material selected from a group including: lead (Pb). Tin (Sn), antimony (Sb), tungsten (W) and bismuth (Bi).

In other examples, the housing portion 791, the spacers 793 and the radioactive inserts can have cross sections selected from among a group including: a circle shape, an oval shape, a polygonal shape, and a nonpolygonal shape.

Referring to FIG. 23B, the side radiating brachytherapy probe 790 is configured to be inserted into the plug lumen 158 of the plug catheter 150 or extended plug catheter 301 and/or one or more channels 742 of the lumen applicator 740 and/or affixed at the distal end of the one or more clips 743 of the inner lumen applicator. An imaging device 751 is inserted through the lumen applicator 740 and is configured to guide and position the side radiating brachytherapy probe 790 to the target tissue 176.

Referring to FIG. 23A, the cylindrical housing 791 is defined by a housing opening width—“A”, a housing opening length “B”, a radioactive insert length “C”, a radioactive insert diameter “D”, a housing diameter “E”, and a space “F” between the end cap 794 and the cylindrical radioactive insert 795, which defines a specific radiation pattern “θ” and “φ” In some examples, cylindrical housing spacers (not shown) are placed distal or proximal to the cylindrical radioactive insert 795, where the spacers can be solid or patterned with one or more holes. The side radiating brachytherapy lumen probe is configured to provide a specified radiation dose to a targeted tissue 176 of the body 170 and is defined by the housing dimensions “A”, “B”, “C”, “D”, “E”, “F”, “θ”, “φ”, the spacers, the measured radiation of the cylindrical radioactive insert 795 at the time of treatment, the distance between the distal surface of the cylindrical radioactive insert 795 and the proximal surface of the target tissue 176, the absorption of radiation by the side wall of the extensible plug catheter 301 and the time the enface radiating brachytherapy lumen probe 790 and plug catheter 301 resides at the target tissue 176. One or more enface radiating lumen brachytherapy probes 790 can be used to create specific radiation intensity patterns dependent on the size and shape of the target tissue 176. The radiation dose can be extended along a targeted tissue 176 of the body 170 by actively repositioning the extensible plug catheter 301 and brachytherapy probe 790 using the imaging device 751. Biosensors 365 on the distal portion end 304 of the extensible plug catheter 301, are configured to monitor the radiation treatment before, during and after the radiation treatment. Data from the biosensors 365 is collected and used by the PPIS 101 and SDT 100 as previously described.

In other examples the brachytherapy needle probe 760, the enface radiating brachytherapy probe 780, the side radiating brachytherapy probe 790, CCM device, TMM devices and CTD devices and the inner lumen applicator as describe above can be configured in one or more combinations and inserted through the PPIS lumen 113 of PPIS 101 in the open configuration of FIG. 1A to treat a target tissue 176 of the body 170.

In some examples, the PPIS 101 can include one or more multiplexing and demultiplexing systems to efficiently manage electrical, optical, fluid, and/or data pathways through the PPIS 101. The multiplexing and demultiplexing system(s) can help to efficiently use the limited space of the port housing 106 to enhance the functional capabilities of the SDT 100 and/or the PPIS 101.

FIG. 24A depicts an implementation of the PPIS 101 that includes a multiplexing and demultiplexing system 901, according to an example. In FIG. 24A, the multiplexing and demultiplexing system 901 is configured to reduce the number of wires and optical fibers that go through the percutaneous body portion 112 of the port housing 106, which allows for a reduction in the size of the percutaneous portion's 112 diameter.

As shown in FIG. 21A, the multiplexing and demultiplexing system 901 can include a plurality electrical wires 941 that are coupled to one or more electrically enabled therapeutic and/or diagnostic devices 940, and/or a plurality optical fibers 943 coupled to one or more electrically enabled therapeutic and/or diagnostic devices 942. The plurality of electrical wires 941 and optical fibers 943 can be coupled through the proximal housing portion 110 to a multiplexor 946. Additional electrical devices 944 and/or optical devices 945 contained within the proximal housing portion 110 can be coupled to the multiplexor 946. The multiplexor 946 can combine and reduce the number of wires and/or optical fibers. The reduced set of wires and/or optical fibers are coupled to a demultiplexer 947 located in the distal housing portion 114. The demultiplexer 947 can distribute the wires and/or the optical fibers to one or more electrical distribution devices 949 and/or one or more optical distribution devices 949 located within the distal housing portion 114. These wires and/or optical fibers are then distributed through one or more electrical connectors 951 and/or one or more optical connectors 952 to a plurality of electrical devices 954 and/or a plurality of optical devices 955 distributed throughout the body cavity 172 and body 170 as seen in FIGS. 1A-1C. The demultiplexer 947 can also distribute the electrical wires and/or optical fibers 950 to the port catheter 104.

The multiplexing demultiplexing system 901 is configured to divide the capacity of a communication channel into several logical channels, one for each electrical and optical message signal or data stream to be transferred through multiplexing and the extraction of the original channels through demultiplexing. The multiplexing demultiplexing system 901 can be configured to be at least one system selected from the group consisting of: a space-division multiplexing system, frequency-division multiplexing system, time-division multiplexing system, wavelength-division multiplexing system, and polarization-division multiplexing system.

Additionally, as shown in FIG. 24A, the PPIS 101 can include one or more thermal bladders 902, 920 and, in some implementations, the PPIS 101 can include a multiplexing and demultiplexing system (e.g., a fluid manifold 914) for a fluid used with the fluid bladder(s) 902, 920. Although FIG. 24A depicts an implementation of the PPIS 101 that includes the fluid bladder(s) 902, 920 and fluid manifold 914, the PPIS 101 can include the fluid bladder(s) 902, 920 without the fluid manifold 914 or the PPIS can include the fluid manifold 914 without the fluid bladder(s) 902, 920 in other examples.

As shown in FIG. 24A, a therapeutic treatment device of the PPIS 101 (and/or the CTD and/or the TMM) can include a thermal circulatory bladder system 900, which includes a fluid bladder 902 configured to be positioned on or adjacent (178, 182) to the tissue 176 in the body cavity 172. The thermal circulatory bladder system 900 can also include a bladder inlet 904 configured to receive a fluid from a temperature-controlled fluid circulatory system 906, and an inlet tube 908 coupling the fluid bladder 902 to the bladder inlet 904. The inlet tube 908 can extend from the bladder inlet 904 at the port housing 106 to the fluid bladder 902. The thermal circulatory bladder system 900 can also include a bladder outlet 910 that is configured to return the fluid from the fluid bladder 902 to the temperature-controlled fluid circulatory system 906, and an outlet tube 912 coupling the fluid bladder 902 to the bladder outlet 910, where the outlet tube 912 extends from the bladder outlet 910 at the port housing 106 to the fluid bladder 902.

In some examples, the inlet tube 908 can include an insulator surrounding the inlet tube 908 along at least a portion of the inlet tube 908 between the port housing 106 and the fluid bladder 902, and the outlet tube 912 can include an insulator surrounding the outlet tube 912 along at least a portion of the outlet tube 912 between the port housing and the fluid bladder. This can help to maintain the fluid at a desired temperature. As examples, the insulator surrounding the inlet tube 908 and the insulator surrounding the outlet tube 912 can include a polymer material. For instance, the insulator surrounding the inlet tube 908 and/or the insulator surrounding the outlet tube 912 and include an insulating jacket filled with an inert gas in one example.

In the example shown in FIG. 24A, the PPIS 101 can further include a fluid manifold 914, and the bladder inlet 904 and the bladder outlet 910 can be coupled to the temperature-controlled fluid circulatory system 906 via the fluid manifold 914 of the PPIS 101. The fluid manifold 914 can be in the proximal housing portion 110 of the port housing 106. The thermal circulatory bladder system 900 can be configured to circulate the fluid from a temperature-controlled fluid circulatory system 906 to the fluid manifold 914, through a manifold inlet 916 of the fluid manifold 914 of the percutaneous port implant system to the bladder inlet 904, through the bladder inlet 904 to the inlet tube 908, through the inlet tube 908 to the fluid bladder 902, through the fluid bladder 902 to the outlet tube 912, through the outlet tube 912 to the bladder outlet 910, through the bladder outlet 910 to the fluid manifold 914, through a manifold outlet 918 of the fluid manifold 914 to the temperature-controlled fluid circulatory system 906.

In the example shown in FIG. 24A, the thermal circulatory bladder system 900 can also include a second bladder 920 configured to be positioned on or adjacent to the same tissue in the body cavity 172 and/or another tissue in the body cavity 172. In some implementations, the second bladder 920 can extend around a circumference of the fluid bladder 902. In other implementations, the second bladder 920 and the fluid bladder 902 can be adjacent to each other and/or remotely spaced apart from each other.

The thermal circulatory bladder system 900 can also include a second bladder inlet 922 configured to receive a second fluid from the temperature-controlled fluid circulatory system 906, a second inlet tube 924 coupling the second bladder 920 to the second bladder inlet 922, where the second inlet tube 924 extends from the second bladder inlet 922 at the port housing 106 to the second bladder 920, a second bladder outlet 926 configured to return the second fluid from the second bladder 920 to the temperature-controlled fluid circulatory system 906, and a second outlet tube 928 coupling the second bladder 920 to the second bladder outlet 926, where the second outlet tube 928 extends from the second bladder outlet 926 at the port housing 106 to the second bladder 920.

In some examples, the second inlet tube 924 can include an insulator surrounding the second inlet tube 924 along at least a portion of the second inlet tube 924 between the port housing 106 and the second bladder 920, and the second outlet tube 928 can include an insulator surrounding the second outlet tube 928 along at least a portion of the second outlet tube 928 between the port housing 106 and the second bladder 920. In addition, the insulator surrounding the second inlet tube 924 and the insulator surrounding the second outlet tube 928 cab be a polymer material and/or an insulating jacket filled with an inert gas.

Additionally, in some examples, the thermal circulatory bladder system 900 can include one or more temperature sensors 909, 911, 905, 913. For instance, the thermal circulatory bladder system 900 can include one or more temperature sensors 909 on a portion of an exterior surface of the fluid bladder 902 that is configured to engage the tissue 176, one or more temperature sensors 905 in a portion of the fluid bladder 902 that is configured to measure the temperature of the fluid of the fluid bladder 902, one or more temperature sensors 911 on a portion of an exterior surface of the second bladder 920 that is configured to engage the tissue 176, and/or one or more temperature sensors 913 in a portion of the second bladder 920 that is configured to measure the temperature of the second fluid of the second bladder 920.

In some implementations, the temperature sensor(s) 909, 911, 905, 913 can provide a signal indicative of a measured temperature that can be used as feedback to control a temperature of the fluid and/or the second fluid supplied by the temperature-controlled fluid circulatory system 906. In some examples, the thermal circulatory bladder system 900 is configured to supply the fluid to the fluid bladder 902 and/or supply the second fluid to the second bladder 920 at a temperature that is configured to raise a temperature of the tissue (176, 178, 182), and/or to maintain the fluid in the fluid bladder 902 and/or the second fluid in the second bladder 920 at a temperature between 98 degrees Fahrenheit (F) to 116 degrees F and 110 degrees F to 116 degrees F. In some examples, the thermal circulatory bladder system 900 can be additionally or alternatively configured to supply the fluid to the fluid bladder 902 at a temperature that is configured to lower a temperature of the tissue (176, 178, 182), and/or maintain the fluid in the fluid bladder 902 at a temperature between 33 degrees Fahrenheit (F) to 98 degrees F and 75 degrees F to 98 degrees F).

In some examples, the first fluid and/or the second fluid can include a saline solution. In another example, the first fluid bladder 902 can be inflated with a gas and the second bladder 920 can be inflated with a liquid of the thermal circulatory bladder system 900. In another example, the second bladder 920 can be inflated with a gas and the first bladder 902 can be inflated with a liquid of the thermal circulatory bladder system 900.

In some examples, the temperature of the fluid in the fluid bladder 902 can be the same as the temperature of the second fluid in the second bladder 920. In other examples, the thermal circulatory bladder system 900 can be configured to supply the first fluid at a first temperature and the second fluid at a second temperature, where the first temperature is different than the second temperature. For instance, in some examples, the temperature of the fluid in the fluid bladder 902 can be greater than the temperature of the second fluid in the second bladder 920. In some examples, the temperature of the fluid in the fluid bladder 902 can be less than the temperature of the second fluid in the second bladder 920.

As noted above, in FIG. 24A, the PPIS 101 can include the fluid manifold 914. As shown in FIG. 24A, the bladder inlet 904 and the bladder outlet 910 can be coupled to the temperature-controlled fluid circulatory system 906 via the fluid manifold 914 of the PPIS 101, and the second bladder inlet 922 and the second bladder outlet 926 can be coupled to the temperature-controlled fluid circulatory system 906 via the fluid manifold 914 of the PPIS 101.

In this example, the thermal circulatory bladder system 900 of the PPIS 101 is configured to circulate the fluid from a temperature-controlled fluid circulatory system 906 to the fluid manifold 914, through a manifold inlet 916 of the fluid manifold 914 of the PPIS 101 to the bladder inlet 904, through the bladder inlet 904 to the inlet tube 908, through the inlet tube 908 to the fluid bladder 902, through the fluid bladder 902 to the outlet tube 912, through the outlet tube 912 to the bladder outlet 910, through the bladder outlet 910 to the fluid manifold 914, through a manifold outlet 918 of the fluid manifold 914 to the a temperature-controlled fluid circulatory system 906. The thermal circulatory bladder system 900 of the PPIS 101 is also configured to circulate the second fluid from the temperature-controlled fluid circulatory system 906 to the fluid manifold 914, through a second manifold inlet 930 of the fluid manifold 914 of the percutaneous port implant system to the second bladder inlet 922, through the second bladder inlet 922 to the second inlet tube 924, through the second inlet tube 924 to the second bladder 920, through the second bladder 920 to the second outlet tube 928, through the second outlet tube 928 to the second bladder outlet 926, through the second bladder outlet 926 to the fluid manifold 914, through a second manifold outlet 932 of the fluid manifold 914 to the a temperature-controlled fluid circulatory system 906.

As shown in FIG. 24A, the temperature-controlled fluid circulatory system 906 of the PPIS 101 can include a first temperature-controlled fluid source 906A that is configured to supply the fluid to the fluid manifold 914 and a second temperature-controlled fluid source 906B that is configured to supply the second fluid to the fluid manifold 914. In an example, the fluid manifold 914 can include: a supply tube 936 and a return tube 937 that each extend, through the percutaneous body portion 112, between the proximal housing portion 110 and the distal housing portion 114. Also, in the example shown in FIG. 24A, the fluid manifold 914 can include a plurality of valves 934A-934H that are operable to: (i) supply, via the supply tube 936, the fluid from the first temperature-controlled fluid source 906A to the fluid bladder 902, (ii) supply, via the supply tube 936, the fluid from the first temperature-controlled fluid source 906A to the second bladder 920, (iii) supply, via the supply tube 936, the second fluid from the second temperature-controlled fluid source 906B to the fluid bladder 902, and (iv) supply, via the supply tube 936, the second fluid from the second temperature-controlled fluid source 906B to the second bladder 920. The plurality of valves 934A-934H can be further operable to: (v) return, via the return tube 937, the fluid from the first fluid bladder 902 to the first temperature-controlled fluid source 906A, (vi) return, via the return tube 937, the fluid from the second bladder 920 to the first temperature-controlled fluid source 906A, (vii) return, via the return tube 937, the second fluid from the first fluid bladder 902 to the second temperature-controlled fluid source 906B, and (viii) return, via the return tube 937, the second fluid from the second bladder 920 to the second temperature-controlled fluid source 906B.

Additionally, in this example, the plurality of valves 934A-934H of the temperature-controlled fluid circulatory system 906 can include a first valve 934A that is fluidly coupled to the bladder inlet 904, a second valve 934B that is fluidly coupled to the bladder outlet 910, a third valve 934C that is fluidly coupled to the second bladder inlet 922, and a fourth valve 934D that is fluidly coupled to the second bladder outlet 926. The plurality of valves 934A-934H can also include a fifth valve 934E that is fluidly coupled to the manifold inlet 916, a sixth valve 934F that is fluidly coupled to the manifold outlet 918, a seventh valve 934G that is fluidly coupled to the second manifold inlet 930, and an eighth valve 934H that is fluidly coupled to the second manifold outlet 932.

Additionally, the supply tube 936 can include a proximal end at the proximal housing portion 110 and a distal end at the distal housing portion 114, the return tube 937 can include a proximal end at the proximal housing portion 110 and a distal end at the distal housing portion 114, and the plurality of valves can be further configured to: (i) open and close the proximal end and the distal end of the supply tube 936, and (ii) open and close the proximal end and the distal end of the return tube 937.

In some examples, the fluid manifold 914 and/or the valves 934A-934H can additionally or alternatively couple to the port tube 116 to one or more channels 602 and 630 as seen in FIG. 6C. In some examples, the fluid bladder 902 and the second bladder 920 can be configured to be inflated through an inflation port 907A coupled to the fluid manifold 914 through a connector 939A and a deflation port 907B that is coupled to the fluid manifold 914 through a connector 939B. In some implementations, the inflation port 907A and the deflation port 907B can be configured to couple to a gas to facilitate the first fluid and/or the second fluid being a gas.

Additionally, as shown in FIG. 24A, the fluid manifold 914 can include one or more fluid valves 935A-935D that control the flow of the fluid(s) between the (i) the supply valves 934A-934D and (ii) the supply valves 934E-934G. For instance, the fluid valve 935A and/or the fluid valve 935C can be configured to control a flow of fluid(s) through a first fluid conduit extending through the percutaneous body portion 112, and the fluid valve 935B and/or the fluid valve 935D can be configured to control a flow of fluid(s) through a second fluid conduit extending through the percutaneous body portion 112.

FIG. 24B depicts another implementation of the PPIS 101 including another implementation of the thermal circulatory bladder system 900, according to another example. In FIG. 24B, one or more components of the thermal circulatory bladder system 900 are included in the plug catheter 150.

As shown in FIG. 24B, the PPIS 101 can include the one or more thermal bladders 902, 920 described above. Additionally, for example, as shown in FIG. 24B, the plug catheter 150 can include the inlet tube 908, the outlet tube 912, the second inlet tube 924, and the second outlet tube 928. The bladder inlet 904, the bladder outlet 910, the second bladder inlet 922, and the second bladder outlet 926 can be at the proximal plug end 152 of the plug catheter 150. In some examples, the inlet tube 908, the second bladder inlet 922, the bladder outlet 910, and/or the second bladder outlet 926 can include an insulator as described above. Additionally, the thermal circulatory bladder system 900 can include the one or more temperature sensors 909, 911, 905, 913, as described above. The thermal circulatory bladder system 900 can additionally or alternatively be configured to supply the first fluid and/or the second fluid at the temperatures, and/or to achieve target tissue temperatures as described above.

In some implementations, the plug catheter 150 and the thermal circulatory bladder system 900 can configured to deliver a photosensitizer and/or supply oxygen to a target tissue (176, 178, 182) at the distal port catheter end 118 or the distal portion end 304 through the self-healing window 202 as previously described. In some examples, the plug catheter 150 can include a needle at the distal plug end 154, and the needle is configured to inject the photosensitizer into the target tissue.

In some examples, the plug catheter 150 can include one or more optical illuminators 915, 917, 919 that is configured to deliver light to the target tissue 176, 178, 182 at the distal plug end 154 through the self-healing window 202, and/or one or more optical illuminators 915, 117, 119 of the fluid bladder 902 and/or the fluid bladder 920. In some implementations, the shape and surfaces of the fluid bladder 902 and the second bladder 920 can be optically reflective and/or optically diffusive and patterned to deliver a light irradiation distribution pattern to the target tissue 176, 178, 182.

In some examples of the implementation shown in FIG. 24B, the plug catheter 150 can be configured to supply oxygen to the target tissue. In other implementations, the plug catheter 150 can be configured to supply the oxygen and the light to the target tissue simultaneously.

In some examples, the plug catheter 150 can include one or more sensors (e.g., the temperature sensors 909, 911, 905, 913) that are configured to sense parameters related to photodynamic therapy applied to the target tissue (176, 178, 182) through the optical healing window 202, the fluid bladder 902 and/or the second bladder 920. The sensor(s) can be configured to communicate the parameter to a processor (e.g., the AIS) to cause the processor to perform, based on the parameter, feedback control of a light source, an oxygen source, a source of the photosensitizer, the fluid bladder 902, and/or the second bladder 920. As examples, the sensors of the plug catheter 150 can include sensors that are configured to sense parameters related to photodynamic therapy applied to the target tissue (176, 178, 182) selected from the group consisting of: a temperature, an oxygen concentration, an irradiation energy, an irradiation energy distribution, a dose, an interstitial fluid pressure, a pH, a photosensitizer concentration, a bioavailability parameter, a pharmacodynamic parameter, a pharmacokinetic parameter and a biomarker.

As shown in FIG. 24B, in some implementations, the plug catheter 150 can include an insulator 921 between the fluid bladder 902 and the second bladder 920 along at least a portion of the fluid bladder 902 and the second bladder 920 between the external surface of the plug catheter 150 and the tissue (176, 178, 182). Additionally, as shown in FIG. 24B, the plug catheter 150 can additionally or alternatively include an insulator 923 between the second bladder 920 and the body cavity 172 along at least a portion of the fluid bladder 902 and the second bladder 920 between the external surface of the plug catheter 150 and the tissue (176, 178, 182). As examples, the insulators 921 and 923 can include a polymer material and/or an insulating jacket filled with an inert gas.

Referring back to FIG. 5, the PPIS 101 can includes one or more temperature sensors (e.g., sensor(s) 530A, 523B, 530C) coupled to at least one portion of the port housing 106 selected from a group consisting of: the proximal housing portion 110, the distal housing portion 114, and the percutaneous body portion 112. As an example, the temperature sensor(s) can include a first temperature sensor 530A coupled to the proximal housing portion 110, a second temperature sensor 530B coupled to the percutaneous body portion 112, a third temperature sensor 530C coupled to the distal housing portion 114, a fourth temperature sensor coupled to the port catheter 104, and/or a fifth temperature sensor coupled to the plug catheter 150

Within examples, the temperature sensor(s) 530A-530C can be communicatively coupled to a processor 130. In one implementation, the processor 130 can configured to: receive, from the one or more temperature sensors 530A-530C, an indication of a temperature measured by the one or more temperature sensors 530A-530C, determine, based on the temperature measured by the one or more temperature sensors 530A-530C, that a tissue infection is present adjacent to the port housing 106. In another implementation, the processor 130 can be configured to: receive, from the one or more temperature sensors 530A-530C, an indication of a temperature measured by the one or more temperature sensors, determine, based on the temperature measured by the one or more temperature sensors, a core body temperature of the human body 170.

As a further example, the one or more temperature sensors 530A-530C include a plurality of temperature sensors, where the plurality of temperature sensors 530A-530C are communicatively coupled to a processor 130. The processor 130 is configured to: receive, from one temperature sensor of the plurality of temperature sensors 530A-530C, an indication of a first temperature measured at a first location on the port housing 106, receive, from another temperature sensor of the plurality of temperature sensors 530A-530C, an indication of a second temperature measured at a second location on the port housing 106, determine a difference between the first temperature and the second temperature, and determine, based on the difference between the first temperature and the second temperature, that an infection is present in the human body at the port housing 106.

As described above, to access the body cavity 172 for monitoring and/or treating diseases, tissues, and/or environmental conditions, the plug catheter 150 can be inserted in (i) the PPIS lumen 113 defined by the port housing 106 and the port catheter 104 in implementations that include the port catheter 104, or (ii) the PPIS lumen 113 defined by only the port housing 106 in implementations that omit the port catheter 104. In other examples, the PPIS 101 can be configured to provide for accessing the body cavity 172 to monitor and/or treat diseases, tissues, and/or environmental conditions without using the plug catheter 150.

For instance, the PPIS 101 can include the closed window 202 at the distal port catheter end 118 (e.g., at the collar 219 shown in FIG. 2A and/or at the distal portion end 304 of the extensible plug catheter 301 shown in FIGS. 15A-15B) and/or the PPIS 101 can include the closed window 202 at the distal opening 109 of the port housing 106. In these implementations, any of the device described above with respect to the plug catheter 150 (e.g., an imaging device, endoscope, an active steering device, an injection device, a biopsy device, a surgical tool, an irrigation device, an insufflation device, a deflation device, an ultrasonic device, an ablation device, a light delivery device, a thermal delivery device, a radiation device, a radiation seed delivery device, a biosensor, and/or any of the devices described below with respect to other systems of the SDT 100) can be directly inserted in the PPIS lumen 113 and positioned at and/or through the closed window 202. The configurations can eliminate the third route of biofilm formation on the internal surface of the catheter from ever being in direct contact with the body while still protecting against tunnel infection and the external surface of the catheter.

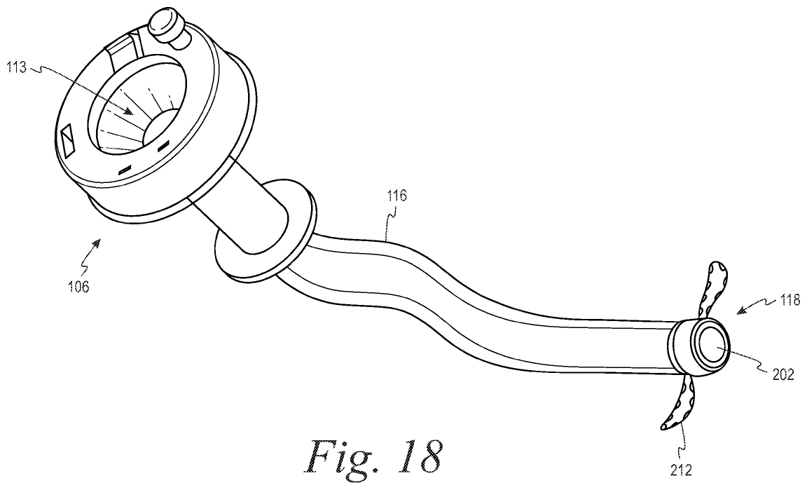

FIG. 18 depicts an implementation in which the PPIS 101 includes the closed window 202 at the distal port catheter end 118 of the port catheter 104, and the port housing 106 and the port catheter 104 are otherwise configured to receive any of the devices described above.

The description of the different advantageous arrangements has been presented for purposes of illustration and description, and is not intended to be exhaustive or limited to the embodiments in the form disclosed. Many modifications and variations will be apparent to those of ordinary skill in the art. Further, different advantageous embodiments may describe different advantages as compared to other advantageous embodiments. The embodiment or embodiments selected are chosen and described in order to explain the principles of the embodiments, the practical application, and to enable others of ordinary skill in the art to understand the disclosure for various embodiments with various modifications as are suited to the particular use contemplated.

What is claimed is:

1. A percutaneous port implant system, comprising:
a port housing comprising:
a proximal housing portion defining a proximal opening at a proximal end of the port housing,
a distal housing portion defining a distal opening at a distal end of the port housing,
a percutaneous body portion extending between the proximal housing portion and the distal housing portion, wherein the percutaneous body portion is configured to engage a wall of a body cavity of a human body while the proximal housing portion is positioned external to the body cavity and the distal housing portion is positioned within an interior of the body cavity when the port housing is implanted in the human body, and
a port lumen extending between the proximal opening and the distal opening, wherein the port lumen extends entirely through the proximal housing portion, the percutaneous body portion, and the distal housing portion; and
a port catheter coupled to the port housing and extending within the port lumen of the port housing,
wherein the port catheter comprises a port tube that defines an outer lumen that extends between a proximal port catheter end and a distal port catheter end, and
wherein the port lumen of the port housing and the outer lumen of the port catheter define a percutaneous port implant system (PPIS) lumen that extends between the proximal opening of the port housing and the distal port catheter end.

2. The percutaneous port implant system of claim 1, wherein the proximal port catheter end is fixedly coupled to the distal housing portion, and wherein the distal port catheter end extends distally of the distal housing portion.

3. The percutaneous port implant system of claim 2, wherein the distal port catheter end is configured to be sutured to a target tissue in the body cavity.

4. The percutaneous port implant system of claim 1, wherein the port catheter comprises an elastic material such that the port tube is configured to collapse on itself when the outer lumen is unoccupied and radially expand when the outer lumen is occupied.

5. The percutaneous port implant system of claim 4, wherein the port catheter further comprises a stiffener that resists the port tube stretching in an axial direction between the proximal port catheter end and the distal port catheter end while allowing the port tube to collapse on itself when the outer lumen is unoccupied and radially expand when the outer lumen is occupied.

6. The percutaneous port implant system of claim 1, further comprising a plug catheter that is configured to be inserted in the outer lumen of the port catheter, wherein the plug catheter comprises a plug tube that defines an plug lumen extending between a proximal plug end and a distal plug end.

7. The percutaneous port implant system of claim 6, wherein the plug catheter is configured to be inserted in the PPIS lumen such that the distal plug end extends to the distal port catheter end.

8. The percutaneous port implant system of claim 6, wherein the distal plug end comprises a closed window that is configured to seal the plug lumen from the body cavity.

9. The percutaneous port implant system of claim 8, wherein the closed window comprises a self-healing window that is configured to (i) form seal around a device in the plug lumen that pierces through the closed window at a puncture site and (ii) reseal the closed window at the puncture site responsive to the device ceasing to pierce through the closed window.

10. The percutaneous port implant system of claim 9, wherein the device comprises at least one device selected from a group consisting of: an imaging device, endoscope, an active steering device, an injection device, a biopsy device, a surgical tool, an irrigation device, an insufflation device, a deflation device, an ultrasonic device, an ablation device, a light delivery device, a thermal delivery device, a radiation device, a radiation seed delivery device, a biosensor.

11. The percutaneous port implant system of claim 8, wherein the closed window is configured to allow an imaging device to capture an image through the closed window.

12. The percutaneous port implant system of claim 6, wherein the plug catheter is configured to be inserted in the port catheter such that the distal plug end extends distally of the distal port catheter end.

13. The percutaneous port implant system of claim 12, wherein the plug catheter comprises a plurality of holes, wherein the plurality of holes are arranged along a longitudinal axis of a distal portion of the plug catheter, wherein the distal portion of the plug catheter is configured to extend distally of the distal port catheter end.

14. The percutaneous port implant system of claim 6, further comprising:
a proximal sealing element configured to form a fluid-tight seal (i) between an interior surface of the port housing at the proximal housing portion and an outer wall of the plug catheter and (ii) around a circumference of the plug catheter when the plug catheter is inserted in the port lumen of the port housing; and
a distal sealing element configured to form a fluid-tight seal (i) between the distal port catheter end and the plug catheter and (ii) around a circumference of the plug catheter when the plug catheter is inserted in the outer lumen of the port catheter.

15. The percutaneous port implant system of claim 14, wherein the plug catheter comprises a membrane coupled to an outer surface of the plug tube such that the membrane is between the outer surface of the plug tube and an inner surface of the port tube when the plug catheter is inserted in the port catheter, and
wherein the membrane comprises a plurality of electrodes.

16. The percutaneous port implant system of claim 15, further comprising a fluid port inlet and a fluid port outlet,
wherein the inner surface of the membrane and the outer surface of the plug tube define one or more first channels extending along a portion of the plug catheter between the proximal plug end and the distal plug end,
wherein the fluid port inlet is fluidly coupled with the one or more first channels of the plug catheter,
wherein the one or more first channels are fluidly coupled with a second channel between an inner surface of the port catheter and an outer surface of the membrane, and
wherein the fluid port outlet is fluidly coupled to the second channel,
wherein fluid received at the fluid port inlet is configured to flow along the one or more first channels to the second channel, from the second channel to the fluid port outlet, and egress out through the fluid port outlet.

17. The percutaneous port implant system of claim 16, wherein the plurality of electrodes are configured to apply electrical energy to the fluid in the one or more first channels and the second channel to disinfect the plug catheter and the port catheter.

18. The percutaneous port implant system of claim 6, further comprising a protective cap that covers the proximal opening of the port housing and the proximal plug end of the plug catheter.

19. The percutaneous port implant system of claim 1, wherein the proximal housing portion comprises a proximal flange that extends outwardly from the percutaneous body portion, and
wherein the distal housing portion comprises a distal flange that extends outwardly from the percutaneous body portion.

20. The percutaneous port implant system of claim 1, further comprising at least one system selected from a group consisting of:
(i) a continuous cancer monitoring (CCM) system comprising a sensor that is configured to sense a biological parameter related to a tissue in the body cavity, wherein the sensor is configured to access the body cavity via the percutaneous port implant system, wherein the sensor of the CCM system comprises at least one sensor selected from a group consisting of: a biosensor, an imaging sensor, and a molecular assay,
(ii) a tumor microenvironment management (TMM) system comprising an environmental stimulator that is configured to apply one or more stimuli to an environment in which a target tissue is located in the body cavity, wherein the environmental stimulator is configured to access the body cavity via the percutaneous port implant system, wherein the environmental stimulator of the TMM system comprises at least one environmental stimulator selected from a group consisting of: a liquid delivery system, a gas delivery system, a vacuum system, a thermal energy delivery system, a light delivery system, and a radiation delivery system, and
(iii) a cancer therapeutics delivery (CTD) system comprising a therapeutic treatment device that is configured to deliver a therapeutic treatment to a tissue in the body cavity, wherein the therapeutic treatment device is configured to access the body cavity via the percutaneous port implant system, wherein the therapeutic treatment device of the CTD system is configured such that the therapeutic treatment comprises at least one treatment modality selected from a group consisting of: drug therapy, thermal therapy, photodynamic therapy, laser ablation, radiofrequency ablation (RFA), microwave ablation (MWA), high-intensity focused ultrasound (HIFU), irreversible electroporation (IRE), and light-activated nanoparticles ablation therapy.
